(19) United States
(12) Patent Application Publication
(10) Pub. No.: US 2023/0333017 A1
(43) Pub. Date: Oct. 19, 2023

(54) SENSOR WITH LIGHT FILTER AND CROSSTALK REDUCTION MEDIUM

(71) Applicant: ILLUMINA, INC., San Diego, CA (US)

(72) Inventors: Tara BOZORG-GRAYELI, Woodside, CA (US); Ravi BILLA, San Diego, CA (US); Tracy FUNG, San Mateo, CA (US); Mohsen REZAEI, Newark, CA (US); Craig Hetherington, Foster City, CA (US); Paul Sangiorgio, San Diego, CA (US); Craig Ciesia, Mountain View, CA (US); Arvin Emadi, Hayward, CA (US)

(21) Appl. No.: 18/297,891

(22) Filed: Apr. 10, 2023

Related U.S. Application Data
(60) Provisional application No. 63/362,909, filed on Apr. 13, 2022.
(60) Provisional application No. 63/485,400, filed on Feb. 16, 2023.

(51) Int. Cl. G01N 21/64; H01L 27/146

(52) U.S. Cl. CPC G01N 21/6428; H01L 27/14623; G01N 21/6454; H01L 27/14689

(57) ABSTRACT

Provided herein are various examples of aspects of a biosensor and methods for manufacturing and using aspects of a biosensor. The method of manufacturing may include forming a germanium layer above a surface of an image sensor and forming a dielectric stack above a surface of the germanium layer. The biosensor can be utilized by placing nucleic acid(s) in reaction sites of the biosensor, exposing the reaction sites to light from a light source (e.g., excitation light), receiving emitted light from the reaction sites via the germanium layer, and identifying, based on the emitted light, a composition of the one or more nucleic acids.

### CROSS-REFERENCE TO RELATED APPLICATIONS

This application claims priority to U.S. Provisional Patent Application No. 63/362,909, which was filed on Apr. 13, 2022, and to U.S. Provisional Patent Application No. 63/485,400, which was filed on Feb. 16, 2023, which are both incorporated by reference herein in their entirety.

### BACKGROUND

Image sensors are utilized for biological and chemical analysis. Various protocols in biological or chemical research involve performing controlled reactions on local support surfaces or within predefined reaction chambers. The designated reactions may then be observed or detected, and subsequent analysis may help identify or reveal properties of chemicals involved in the reaction. For example, in some multiplex assays, an unknown analyte having an identifiable label (e.g., fluorescent label) may be exposed to thousands of known probes under controlled conditions. Each known probe may be deposited into a corresponding well of a flow cell channel. Observing any chemical reactions that occur between the known probes and the unknown analyte within the wells may help identify or reveal properties of the analyte. Other examples of such protocols include known DNA sequencing processes, such as sequencing-by-synthesis (SBS) or cyclic-array sequencing.

In some conventional fluorescent-detection protocols, an optical system is used to direct an excitation light onto fluorescently labeled analytes and to also detect the fluorescent signals that may emit from the analytes. Such optical systems may include an arrangement of lenses, filters, and light sources. In other detection systems, the controlled reactions occur immediately over a solid-state imager (e.g., charged coupled device (CCD) or a complementary metal-oxide-semiconductor (CMOS) detector) that does not require a large optical assembly to detect the fluorescent emissions.

In some devices that provide fluorescent detection, including in those that utilize several wells (e.g., nanowells) or reaction sites, there may be a risk of crosstalk, where a sensor corresponding to one well or reaction site undesirably receives light from either another well or reaction site or some other source. It may therefore be beneficial, advantageous, and desirable to include features that eliminate or otherwise reduce the risk of such crosstalk. It may also be beneficial, advantageous, and desirable to provide such crosstalk reduction features without undesirably increasing the manufacturing cost or complexity of the device.

### SUMMARY

Accordingly, it may be beneficial to utilize a layer including germanium (e.g., silicon germanium, Si(x)Ge(1-x) or SiₓGe₁₋ₓ), over the aforementioned sensor(s) (e.g., CCD and/or CMOS), for the purpose of loss induced crosstalk reduction (LICR). In various examples herein, resultant sensors (e.g., image sensors) can utilize a layer of germanium as both an emission filter (which blocks excitation light) and for LICR. Thus, in examples herein, one or more layers of germanium are utilized in biosensors to provide semiconductor filtering in addition to LICR.

Thus, shortcomings of the prior art can be overcome and benefits as described later in this disclosure can be achieved through the provision of a method for forming aspects of a biosensor. Various examples of the method are described below, and the method, including and excluding the additional examples enumerated below, in any combination (provided these combinations are not inconsistent), overcome these shortcomings. In some examples herein, the method comprises: forming one or more diodes on a first surface of a substrate, wherein the first surface of the substrate is parallel to a second surface of the substrate; forming one or more trenches between the one or more diodes, the one or more trenches extending toward the second surface of the substrate from the first surface of the substrate, wherein the forming comprises filling the one or more trenches and planarizing the one or more filled trenches to form a first surface substantially parallel to a first surface of the one or more diodes and the first surface of the substrate; removing a portion of the substrate such that the one or more trenches extend through the substrate from the first surface of the substrate to the second surface of the substrate; bonding a carrier wafer to the second surface of the substrate; forming a germanium layer above the second surface of the substrate; and forming a dielectric stack above a surface of the germanium layer.

In some examples of the method, forming the one or more trenches comprises etching the one or more trenches in the substrate.

In some examples of the method, the substrate comprises silicon.

In some examples of the method, filling the one or more trenches comprises filling the one or more trenches with one or more dielectric layers.

In some examples of the method, the dielectric stack comprises one or more nanowells.

In some examples of the method, forming the germanium layer on the second surface of the substrate comprises depositing germanium on the second surface of the substrate.

In some examples of the method, a technique for the depositing is selected from the group consisting of: Plasma Enhanced Chemical Vapor Deposition (PECVD), sputter, e-beam evaporation, crystalline growth and etching, and radical activation bonding in vacuum.

In some examples of the method, the selected technique is crystalline growth and etching and the crystalline growth and etching comprises one of: transfer wafer bonding or direct wafer bonding.

In some examples of the method, forming the germanium layer above the second surface of the substrate further comprises: forming a first one or more dielectric layers on the second surface of the substrate; forming the germanium layer on a surface of the one or more dielectric layers; and forming a second one or more dielectric layers on a surface of the germanium layer.

In some examples of the method, the substrate, one or more diodes, the carrier wafer, and the one or more filled trenches comprise a sensor.

In some examples of the method, the sensor comprises a complementary metal-oxide-semiconductor (CMOS).

In some examples of the method, the germanium layer comprises germanium and silicon.

In some examples of the method, the stack comprises: one or more dielectric layers; and a sensor compatible metal.

In some examples of the method, the germanium layer performs loss induced crosstalk reduction and filters out excitation light when light source emits light toward the dielectric stack.

In some examples of the method, based on the loss induced crosstalk reduction, a signal at neighboring pixels is substantially lower than a signal at paired pixels.

As aforementioned, shortcomings of the prior art can be overcome and benefits as described later in this disclosure can be achieved through the provision of a method for forming aspects of a biosensor. Various examples of the method are described below, and the method, including and excluding the additional examples enumerated below, in any combination (provided these combinations are not inconsistent), overcome these shortcomings. In some examples herein, the method comprises: forming a germanium layer above a top surface of an image sensor; and forming a dielectric stack above a top surface of the germanium layer.

In some examples of the method, the dielectric stack comprises one or more nanowells.

In some examples of the method, forming the germanium layer above the top surface of the image sensor further comprises: forming a first one or more dielectric layers on the top surface of the image sensor; forming the germanium layer on a surface of the one or more dielectric layers; and forming a second one or more dielectric layers on a surface of the germanium layer.

In some examples of the method, forming the germanium layer above the top surface of the image sensor comprises depositing germanium above the top surface of the image sensor.

In some examples of the method, a technique for the depositing is selected from the group consisting of: Plasma Enhanced Chemical Vapor Deposition (PECVD), sputter, e-beam evaporation, crystalline growth and etching, and radical activation bonding in vacuum.

In some examples of the method, the selected technique is crystalline growth and etching and the crystalline growth and etching comprises one of: transfer wafer bonding or direct wafer bonding.

In some examples of the method, the image sensor comprises a complementary metal-oxide-semiconductor (CMOS).

In some examples of the method, the image sensor comprises a backside image sensor with one or more deep trenches.

In some examples of the method, the germanium layer performs loss induced crosstalk reduction and filters out excitation light when light source emits light toward the dielectric stack.

As aforementioned, shortcomings of the prior art can be overcome and benefits as described later in this disclosure can be achieved through the provision of a method for utilizing a biosensor. Various examples of the method are described below, and the method, including and excluding the additional examples enumerated below, in any combination (provided these combinations are not inconsistent), overcome these shortcomings. In some examples herein, the method comprises: obtaining a biosensor, the biosensor comprising: a germanium layer above a top surface of an image sensor; and a dielectric stack above a top surface of the germanium layer, wherein the dielectric stack comprises wells and reaction sites; placing one or more nucleic acids in the reaction sites; and exposing the reaction sites of the biosensor to light from a light source, wherein the light comprises excitation light and emitted light; obtaining, by the image sensor, the emitted light, from the reaction sites, via the germanium layer, the emitted light, wherein the germanium layer filters the excitation light from the light and reduces crosstalk associated with the emitted light; and identifying, by the image sensor, based on the emitted light, a composition of the nucleic acids.

In some examples of the method, the image sensor comprises one or more diodes.

In some examples of the method, the obtaining the emitted light, from the reaction sites, via the germanium layer further comprises: propagating the emitted light through the germanium layer at non-vertical angles to reach at least one diode of the one or more diodes.

In some examples of the method, the reaction sites comprise fluorophores, and wherein based on exposing the reaction sites of the biosensor to light from a light source, the excitation light causes the fluorophores to emit the emitted light.

In some examples of the method, the germanium layer comprises germanium and silicon.

In some examples of the method, the biosensor further comprises: a first one or more dielectric layers on the top surface of the image sensor; and a second one or more dielectric layers on a surface of the germanium layer.

In some examples of the method, the image sensor comprises a complementary metal-oxide-semiconductor (CMOS).

In some examples of the method, the germanium layer comprises germanium and silicon.

In some examples of the method, the dielectric stack comprises: one or more dielectric layers; and a sensor compatible metal.

As aforementioned, shortcomings of the prior art can be overcome and benefits as described later in this disclosure can be achieved through the provision of an apparatus that can be utilized as a biosensor. Various examples of the apparatus are described below, and the apparatus, including and excluding the additional examples enumerated below, in any combination (provided these combinations are not inconsistent), overcome these shortcomings. In some examples herein, the apparatus comprises: a filter layer comprising germanium; a flow channel floor defining a plurality of wells, wherein each well provides a reaction site, wherein the filter layer is positioned under flow channel floor, wherein the filter layer spans contiguously under the plurality of wells.

In some examples, of the apparatus, the apparatus further comprises: a plurality of sensors positioned under the filter layer, each sensor of the plurality of sensors centered under a corresponding well and reaction site, such that each sensor forms a sensing pair with a corresponding reaction site.

In some examples, of the apparatus, the filter layer further comprises silicon.

In some examples, of the apparatus, the filter layer has a height of approximately 300 micrometers to approximately 500 micrometers.

As aforementioned, shortcomings of the prior art can be overcome and benefits as described later in this disclosure can be achieved through the provision of a method for utilizing a biosensor. Various examples of the method are described below, and the method, including and excluding the additional examples enumerated below, in any combination (provided these combinations are not inconsistent), overcome these shortcomings. In some examples herein, the method comprises: placing one or more nucleic acids in reaction sites of an apparatus, the apparatus comprising: a filter layer comprising germanium; a flow channel floor defining a plurality of wells, wherein each well provides a reaction site of the reaction sites, wherein the filter layer is positioned under flow channel floor; wherein the filter layer spans contiguously under the plurality of wells; exposing the reaction sites of the apparatus to light from a light source, wherein the light comprises excitation light and emitted light; receiving the emitted light from the reaction sites via the filter layer, wherein the filter layer filters the excitation light from the light and reduces crosstalk associated with the emitted light; and identifying, based on the emitted light, a composition of the one or more nucleic acids.

In some examples of the method, the apparatus includes a plurality of sensors positioned under the filter layer, each sensor of the plurality of sensors centered under a corresponding well and another reaction site of the reaction sites, such that each sensor forms a sensing pair with a corresponding reaction site.

In some examples of the method, the filter layer of the apparatus further comprises silicon.

In some examples of the method, the filter layer has a height of approximately 300 micrometers to approximately 500 micrometers.

As aforementioned, shortcomings of the prior art can be overcome and benefits as described later in this disclosure can be achieved through the provision of a method for forming aspects of a biosensor. Various examples of the method are described below, and the method, including and excluding the additional examples enumerated below, in any combination (provided these combinations are not inconsistent), overcome these shortcomings. In some examples herein, the method comprises: forming a germanium layer over a top surface of a sensor, wherein the sensor comprises: a substrate comprising one or more diodes; a first oxide layer formed over a top surface of the substrate; forming a first conductive layer over a top surface of the germanium layer; forming a second oxide layer over a top surface of the first conductive layer; forming a second conductive layer over a top surface of the second oxide layer; depositing photoresist on a first portion of a top surface of the second conductive layer; and etching through a second portion of the top surface of the second conductive layer, wherein the photoresist is not deposited on the second portion of the top surface of the second conductive layer, a portion of the second oxide layer, and a portion of the first conductive layer, wherein the etching forms one or more trenches, wherein the one or more trenches are each positioned above at least one diode of the one or more diodes, on a vertical axis extending from a bottom surface of the sensor to the top surface of the second oxide layer.

In some examples of the method, the germanium layer further comprises silicon, and forming the germanium layer comprises sputtering silicon-germanium onto the top surface of the first oxide layer.

In some examples of the method, the one or more trenches comprise nanowells.

In some examples of the method, forming the germanium layer over a top surface of a sensor further comprises: depositing photoresist on a first portion of the top surface of the first oxide layer; etching through a second portion of the top surface of the first oxide layer, wherein the photoresist is not deposited on the second portion of the top surface of the first oxide layer, wherein the etching forms one or more trenches in the first oxide layer; depositing a crosstalk mitigating substance above the first oxide layer, wherein the depositing fills the one or more trenches in the first oxide layer; planarizing the crosstalk mitigating substance such that a portion of the crosstalk mitigating substance forms a contiguous surface with the first portion of the top surface of the first oxide layer; and depositing a layer of silicon germanium on the top surface of the first oxide layer.

In some examples of the method, the crosstalk mitigating substance is selected from the group consisting of: oxide, nitride, and silicon.

In some examples of the method, the method also includes: forming a passivation layer over the first portion of the top surface of the second conductive layer.

In some examples of the method, forming the germanium layer over a top surface of a sensor further comprises: sputtering an additional conductive layer on the top surface of the first oxide layer; depositing photoresist on a first portion of the additional conductive layer, wherein the second portion of the first oxide layer is exposed; removing a second portion of the additional conductive layer with etching, wherein the photoresist is not deposited on the second portion of the additional conductive layer, wherein based on the removing, the top surface of the first oxide layer and the first portion of the additional conductive layer are exposed; and depositing a layer of silicon germanium on the top surface of the first oxide layer.

In some examples of the method, forming the germanium layer over a top surface of the sensor comprises: depositing photoresist on a first portion of the top surface of the first oxide layer; etching through a second portion of the top surface of the first oxide layer, wherein the photoresist is not deposited on the second portion of the top surface of the first oxide layer, wherein the etching forms one or more trenches in the first oxide layer; depositing the germanium layer above the first oxide layer, wherein the depositing partially fills the one or more trenches in the first oxide layer; depositing a crosstalk mitigating substance above the germanium layer, wherein the depositing fills a remainder of the one or more trenches in the first oxide layer; and planarizing the crosstalk mitigating substance such that the top surface of the germanium layer is a contiguous surface comprising a portion of the crosstalk mitigating substance and the first portion of the top surface of the first oxide layer.

In some examples of the method, the crosstalk mitigating substance is selected from the group consisting of: oxide, nitride, and silicon.

In some examples of the method, the sensor is a front-side illuminated complementary metal-oxide semiconductor.

In some examples of the method, the method includes: forming a silicon layer on the top surface of the second oxide layer.

In some examples of the method, the first conductive layer and the second conductive layer are comprised of metal.

In some examples of the method, the first oxide layer comprises electrically conductive materials.

In some examples of the method, the sensor is a back-side illuminated complementary metal-oxide semiconductor.

As aforementioned, shortcomings of the prior art can be overcome and benefits as described later in this disclosure can be achieved through the provision of a method for forming aspects of a biosensor. Various examples of the method are described below, and the method, including and excluding the additional examples enumerated below, in any combination (provided these combinations are not inconsistent), overcome these shortcomings. In some examples herein, the method comprises: forming a germanium layer over a top surface of a sensor, wherein the sensor comprises: a substrate comprising one or more diodes; a first oxide layer formed over a top surface of the substrate, wherein forming the germanium layer comprises: depositing photoresist on a first portion of a top surface of the germanium layer; and etching through a second portion of the top surface of the germanium layer, wherein the photoresist is not deposited on the second portion of the top surface of the germanium, wherein the etching forms one or more trenches, wherein the trenches are each positioned above a space between at least one diode and another diode of the one or more diodes, on a vertical axis extending from a bottom surface of the sensor to the top surface of the germanium layer; forming a second oxide layer over a top surface of the germanium layer; depositing photoresist on a first portion of a top surface of the second oxide layer; and etching through a second portion of the top surface of the second oxide layer, wherein the photoresist is not deposited on the second portion of the top surface of the second oxide layer, wherein the etching forms an additional one or more trenches, wherein the additional one or more trenches are each positioned above at least one diode of the one or more diodes, on a vertical axis extending from a bottom surface of the sensor to the top surface of the second oxide layer.

In some examples of the method, the method includes: forming a silicon layer over the top surface of the second oxide layer.

In some examples of the method, forming the germanium layer over the top surface of the sensor further comprises: depositing silicon germanium on the top surface of the first oxide layer; depositing a conductive layer on the silicon germanium; and depositing photoresist on a first portion of the conductive layer; and etching through a second portion of the conductive layer, wherein the photoresist is not deposited on the second portion of the conductive layer, wherein the etching removes the second portion of the conductive layer, and wherein the top surface germanium layer comprises a surface comprising a portion of the silicon germanium and the first portion of the conductive layer.

In some examples of the method, the method includes: depositing a conductive layer on the top surface of the second oxide layer; depositing photoresist on a first portion of the conductive layer; and etching through a second portion of the conductive layer, wherein the photoresist is not deposited on the second portion of the conductive layer, wherein the etching removes the second portion of the conductive layer.

In some examples of the method, forming the germanium layer over the top surface of the sensor further comprises: depositing silicon germanium on the top surface of the first oxide layer; depositing a conductive layer on the top surface of the silicon germanium; depositing photoresist on a first portion of the conductive layer; and etching through a second portion of the conductive layer, wherein the photoresist is not deposited on the second portion of the conductive layer, wherein the etching removes the second portion of the conductive layer, and where the top surface of the germanium layer comprises the first portion of the conductive layer and a portion of the silicon germanium.

In some examples of the method, forming the germanium layer over the top surface of the sensor further comprises: depositing a conductive layer on the top surface of the sensor; depositing photoresist on a first portion of the conductive layer; and etching through a second portion of the conductive layer, wherein the photoresist is not deposited on the second portion of the conductive layer, wherein the etching removes the second portion of the conductive layer; and depositing silicon germanium over a portion of the first oxide layer and the first portion of the conductive layer.

In some examples of the method, the sensor is a front-side illuminated complementary metal-oxide semiconductor.

In some examples of the method, the sensor is a back-side illuminated complementary metal-oxide semiconductor.

In some examples of the method, the first oxide layer comprises conductive components.

As aforementioned, shortcomings of the prior art can be overcome and benefits as described later in this disclosure can be achieved through the provision of an apparatus comprising a biosensor. Various examples of the apparatus are described below, and the apparatus, including and excluding the additional examples enumerated below, in any combination (provided these combinations are not inconsistent), overcome these shortcomings. In some examples herein, the apparatus comprises: a sensor comprising: a substrate comprising one or more diodes; a first oxide layer formed over a top surface of the substrate; a germanium layer formed over a top surface of the sensor; a first conductive layer formed over a top surface of the germanium layer; a second oxide layer formed over a top surface of the first conductive layer; a second conductive layer formed over a top surface of the second oxide layer, wherein the second conductive layer, the second oxide layer, and the first conductive layer comprise one or more trenches, and wherein the one or more trenches are each positioned above at least one diode of the one or more diodes, on a vertical axis extending from a bottom surface of the sensor to the top surface of the second oxide layer.

In some examples of the apparatus, the germanium layer further comprises silicon.

In some examples of the apparatus, the one or more trenches comprise nanowells.

In some examples of the apparatus, the first oxide layer comprises an oxide substance and a crosstalk mitigating substance, wherein the crosstalk mitigating substance fills trench structures in the oxide substance.

In some examples of the apparatus, the crosstalk mitigating substance is selected from the group consisting of: oxide, nitride, and silicon.

In some examples of the apparatus, the apparatus includes: a passivation layer formed over the first portion of the top surface of the second conductive layer.

In some examples of the apparatus, the germanium layer comprises: an additional conductive layer over the top surface of the first oxide layer, wherein the additional and conductive layer comprises fissures; and a layer of silicon germanium over the top surface of the first oxide layer.

In some examples of the apparatus, the first oxide layer comprises trench structures, wherein the top surface of the sensor is an uneven surface and the germanium layer formed over a top surface of the sensor comprises: silicon germanium filling a portion of the trench structures; and a crosstalk mitigating substance filling a remainder of the trench structures, wherein the top surface of the germanium layer is a contiguous surface comprising a portion of the crosstalk mitigating substance and a portion of a top surface of the first oxide layer.

In some examples of the apparatus, the crosstalk mitigating substance filling the remainder of the trench structures is selected from the group consisting of: oxide, nitride, and silicon.

In some examples of the apparatus, the sensor is a front-side illuminated complementary metal-oxide semiconductor.

In some examples of the apparatus, the oxide layer comprises electrically conductive materials.

In some examples of the apparatus, the apparatus includes: a silicon layer on the top surface of the second oxide layer.

In some examples of the apparatus, the first conductive layer and the second conductive layer are comprised of metal.

In some examples of the apparatus, the sensor is a back-side illuminated complementary metal-oxide semiconductor.

In some examples of the apparatus, the apparatus includes a pixel pitch of less than one micron.

In some examples of the apparatus, the germanium layer is of a thickness of less than 400 nm.

In some examples of the apparatus, the germanium layer is of a thickness between approximately 300 nm and approximately 330 nm.

As aforementioned, shortcomings of the prior art can be overcome and benefits as described later in this disclosure can be achieved through the provision of an apparatus comprising a biosensor. Various examples of the apparatus are described below, and the apparatus, including and excluding the additional examples enumerated below, in any combination (provided these combinations are not inconsistent), overcome these shortcomings. In some examples herein, the apparatus comprises: a sensor comprising: a substrate comprising one or more diodes; and a first oxide layer formed over a top surface of the substrate; a germanium layer over a top surface of a sensor, wherein the germanium layer comprises one or more trenches positioned above a space between at least one diode and another diode of the one or more diodes, on a vertical axis extending from a bottom surface of the sensor to the top surface of the germanium layer; and a second oxide layer over a top surface of the germanium layer, wherein the second oxide layer fills the trenches in the germanium layer, wherein the second oxide layer comprises one or more trenches, each trench in the second oxide layer positioned above at least one diode of the one or more diodes, on a vertical axis extending from a bottom surface of the sensor to a top surface of the second oxide layer, wherein the trenches in the second oxide layer expose portions of the germanium layer.

In some examples of the apparatus, the apparatus includes: a silicon layer over the top surface of the second oxide layer.

In some examples of the apparatus, the apparatus includes: a conductive layer comprising lining the one or more trenches in the germanium layer.

In some examples of the apparatus, the apparatus includes: a conductive layer over the top surface of the second oxide layer.

In some examples of the apparatus, the apparatus includes: a conductive layer over the top surface of the sensor.

In some examples of the apparatus, the sensor is a front-side illuminated complementary metal-oxide semiconductor.

In some examples of the apparatus, the first oxide layer comprises conductive components.

In some examples of the apparatus, the sensor is a back-side illuminated complementary metal-oxide semiconductor.

In some examples of the apparatus, the apparatus includes: a pixel pitch of less than one micron.

In some examples of the apparatus, the germanium layer is of a thickness of less than 400 nm.

In some examples of the apparatus, the germanium layer is of a thickness of less than 300 nm.

In some examples of the apparatus, the germanium layer is of a thickness between approximately 300 nm and approximately 330 nm.

As aforementioned, shortcomings of the prior art can be overcome and benefits as described later in this disclosure can be achieved through the provision of a method for utilizing a biosensor. Various examples of the method are described below, and the method, including and excluding the additional examples enumerated below, in any combination (provided these combinations are not inconsistent), overcome these shortcomings. In some examples herein, the method comprises: placing one or more nucleic acids in reaction sites of a sensor, the sensor comprising: a substrate comprising one or more diodes; and a first oxide layer formed over a top surface of the substrate; a germanium layer over a top surface of the first oxide layer, wherein the germanium layer comprises one or more trenches positioned above a space between at least one diode and another diode of the one or more diodes, on a vertical axis extending from a bottom surface of the sensor to the top surface of the germanium layer; and a second oxide layer over a top surface of the germanium layer, wherein the second oxide layer fills the trenches in the germanium layer, wherein the second oxide layer comprises one or more trenches, each trench in the second oxide layer positioned above at least one diode of the one or more diodes, on a vertical axis extending from a bottom surface of the sensor to a top surface of the second oxide layer, wherein the trenches in the second oxide layer expose portions of the germanium layer, wherein the second oxide layer comprises wells and the reaction sites; exposing the reaction sites of the sensor to light from a light source, wherein the light comprises excitation light and emitted light; receiving, by the one or more diodes, the emitted light from the reaction sites via the germanium layer, wherein the germanium layer filters the excitation light from the light and reduces crosstalk associated with the emitted light; and identifying, based on the emitted light, a composition of the nucleic acids.

In some examples of the method, the sensor further comprises: a conductive layer on the top surface of the sensor.

In some examples of the method, receiving the emitted light from the reaction sites via the germanium layer further comprises: propagating the emitted light through the germanium layer to reach at least one diode of the one or more diodes.

In some examples of the method, the reaction sites comprise fluorophores, and wherein based on exposing the reaction sites of the sensor to light from a light source, the excitation light causes the fluorophores to emit the emitted light.

In some examples of the method, the germanium layer comprises germanium and silicon.

In some examples of the method, the sensor is a front-side illuminated complementary metal-oxide semiconductor.

In some examples of the method, the sensor is a back-side illuminated complementary metal-oxide semiconductor.

In some examples of the method, the sensor further comprises: a silicon layer over the top surface of the second oxide layer.

In some examples of the method, the sensor further comprises: a conductive layer comprising lining the one or more trenches in the germanium layer.

In some examples of the method, the sensor further comprises: a conductive layer over the top surface of the second oxide layer.

As aforementioned, shortcomings of the prior art can be overcome and benefits as described later in this disclosure can be achieved through the provision of a method for utilizing a biosensor. Various examples of the method are described below, and the method, including and excluding the additional examples enumerated below, in any combination (provided these combinations are not inconsistent), overcome these shortcomings. In some examples herein, the method comprises: placing one or more nucleic acids in reaction sites of a biosensor, the biosensor comprising: a sensor, the sensor comprising: a substrate comprising one or more diodes; and a first oxide layer formed over a top surface of the substrate; a germanium layer formed over a top surface of the sensor; a first conductive layer formed over a top surface of the germanium layer; a second oxide layer formed over a top surface of the first conductive layer; a second conductive layer formed over a top surface of the second oxide layer, wherein the second conductive layer, the second oxide layer, and the first conductive layer comprise one or more trenches, and wherein the one or more trenches are each positioned above at least one diode of the one or more diodes, on a vertical axis extending from a bottom surface of the sensor to the top surface of the second oxide layer, wherein the trenches comprise wells and reaction sites; exposing the reaction sites of the biosensor to light from a light source, wherein the light comprises excitation light; receiving, by the one or more diodes, the emitted light from the reaction sites via the germanium layer, wherein the germanium layer filters the excitation light and reduces crosstalk associated with the emitted light; and identifying, based on the emitted light, a composition of the nucleic acids.

In some examples of the method, the germanium layer further comprises silicon.

In some examples of the method, the first oxide layer comprises an oxide substance and a crosstalk mitigating substance, and the crosstalk mitigating substance fills trench structures in the oxide substance.

In some examples of the method, the crosstalk mitigating substance is selected from the group consisting of: oxide, nitride, and silicon.

In some examples of the method, the biosensor further comprises: a passivation layer formed over the first portion of the top surface of the second conductive layer.

In some examples of the method, the germanium layer comprises: an additional conductive layer over the top surface of the first oxide layer, wherein the additional and conductive layer comprises fissures; and a layer of silicon germanium over the top surface of the first oxide layer.

In some examples of the method, the first oxide layer comprises trench structures, wherein the top surface of the sensor is an uneven surface and the germanium layer formed on a top surface of the sensor comprises: silicon germanium filling a portion of the trench structures; and a crosstalk mitigating substance filling a remainder of the trench structures, wherein the top surface of the germanium layer is a contiguous surface comprising a portion of the crosstalk mitigating substance and a portion of a top surface of the first oxide layer.

In some examples of the method, the crosstalk mitigating substance filling the remainder of the trench structures is selected from the group consisting of: oxide, nitride, and silicon.

In some examples of the method, the sensor is a front-side illuminated complementary metal-oxide semiconductor.

In some examples of the method, the oxide layer comprises electrically conductive materials.

In some examples of the method, the sensor is a back-side illuminated complementary metal-oxide semiconductor.

In some examples of the method, the obtaining the emitted light from the reaction sites, via the germanium layer further comprises: propagating the emitted light through the germanium layer to reach at least one diode of the one or more diodes.

In some examples of the method, the reaction sites comprise fluorophores, and wherein based on exposing the reaction sites of the sensor to light from a light source, the excitation light causes the fluorophores to emit the emitted light.

Additional features are realized through the techniques described herein. Other examples and aspects are described in detail herein and are considered a part of the claimed aspects. These and other objects, features and advantages of this disclosure will become apparent from the following detailed description of the various aspects of the disclosure taken in conjunction with the accompanying drawings.

It should be appreciated that all combinations of the foregoing aspects and additional concepts discussed in greater detail below (provided such concepts are not mutually inconsistent) are contemplated as being part of the inventive subject matter and to achieve the advantages disclosed herein.

### BRIEF DESCRIPTION OF THE DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIG. 1 is an example of a biosensor where cross talk reduction is handled by a curtain structure;

FIG. 2 illustrates a configuration of a biosensor that uses a lossy material as both an excitation filter and as a medium to reduce crosstalk;

FIG. 3 depicts a biosensor, which can be utilized as a submicron image sensor, in which a filter layer that provides loss induced crosstalk reduction (LICR) is comprised of germanium;

FIG. 4 depicts a cross-sectional view of the biosensor of FIG. 4;

FIG. 5 depicts a top view of the biosensor of FIG. 4;

FIG. 6 is a plot that illustrates that germanium has a very high absorption difference between the wavelengths of red and green;

FIG. 7 is a plot that illustrates the absorption coefficients of various compounds of silicon germanium which can be utilized in the biosensor examples described herein;

FIG. 8 depicts an example of a fabrication process for a biosensor that includes a germanium layer for both LICR and filtering;

FIG. 9 depicts a workflow for forming a germanium layer in a biosensor;

FIGS. 10A-10B, referred to collectively as FIG. 10, illustrate aspects of various methods fabricating and manufacturing biosensors that include a germanium layer;

FIG. 11 depicts a workflow for manufacturing or fabricating a biosensor that includes a germanium layer for LICR and filtering; this workflow uses a commercially available or off-the-shelf image sensor;

FIG. 12 depicts a workflow and illustrates a biosensor at various stages, where the workflow is for fabricating the biosensor and the resultant biosensor includes a germanium layer for LICR and filtering and an off-the-shelf sensor;

FIG. 13 is an example of an existing sensor that can be integrated into the biosensors described herein;

FIG. 14 is an example of an existing sensor that can be integrated into the biosensors described herein;

FIG. 15 is an example of a general workflow that includes aspects integrated into examples of the methods for forming a biosensor disclosed herein;

FIG. 16 is an example of a general workflow that includes aspects integrated into examples of the methods for forming a biosensor disclosed herein;

FIG. 17 illustrates some examples of biosensors that can be formed with the methods discussed herein; these examples utilize a front-side illuminated sensor and vary in crosstalk mitigation materials integrated into a layer with a low refraction index;

FIG. 18 is an illustration of various workflows that can be followed to form the biosensors illustrated in FIG. 17;

FIG. 19 illustrates some examples of biosensors that can be formed with the methods discussed herein; these examples utilize a front-side illuminated sensor, vary in crosstalk mitigation materials integrated into a layer with a low refraction index, and includes an additional conductive (blocking) layer;

FIG. 20 is an illustration of various workflows that can be followed to form the biosensors illustrated in FIG. 19;

FIG. 21 illustrates some examples of biosensors that can be formed with the methods discussed herein; these examples include an LICR layer that is embedded in a front-side illuminated sensor;

FIG. 22 is an illustration of various workflows that can be followed to form the biosensors illustrated in FIG. 21;

FIG. 23 illustrates some examples of biosensors that can be formed with the methods discussed herein; these examples include an etched LICR layer, as opposed to a blanket LICR layer and include a front-side illuminated sensor;

FIG. 24 is an illustration of various workflows that can be followed to form the biosensors illustrated in FIG. 23;

FIG. 25 illustrates some examples of biosensors that can be formed with the methods discussed herein; these examples include an etched LICR layer, as opposed to a blanket LICR layer, a front-side illuminated sensor, and an additional conductive layer that serves to assist in blocking crosstalk within the biosensors;

FIG. 26 is an illustration of various workflows that can be followed to form the biosensors illustrated in FIG. 25;

FIG. 27 illustrates some examples of biosensors that can be formed with the methods discussed herein; these examples utilize a back-side illuminated sensor and a blanket LICR layer;

FIG. 28 is an illustration of various workflows that can be followed to form the biosensors illustrated in FIG. 27;

FIG. 29 illustrates some examples of biosensors that can be formed with the methods discussed herein; these examples utilize a back-side illuminated sensor and an etched LICR layer;

FIG. 30 is an illustration of various workflows that can be followed to form the biosensors illustrated in FIG. 29; and

FIG. 31 is an illustration of various workflows that can be followed to utilize various apparatuses formed utilizing the methods described herein.

### DETAILED DESCRIPTION

The accompanying figures, in which like reference numerals refer to identical or functionally similar elements throughout the separate views and which are incorporated in and form a part of the specification, further illustrate the present implementation and, together with the detailed description of the implementation, explain the principles of the present implementation. As understood by one of skill in the art, the accompanying figures are provided for ease of understanding and illustrate aspects of certain examples of the present implementation. The implementation is not limited to the examples depicted in the figures.

The terms “connect,” “connected,” “contact” “coupled” and/or the like are broadly defined herein to encompass a variety of divergent arrangements and assembly techniques. These arrangements and techniques include, but are not limited to (1) the direct joining of one component and another component with no intervening components therebetween (i.e., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (notwithstanding the presence of one or more additional components therebetween). It is to be understood that some components that are in direct physical contact with one another may or may not be in electrical contact and/or fluid contact with one another. Moreover, two components that are electrically connected, electrically coupled, optically connected, optically coupled, fluidly connected or fluidly coupled may or may not be in direct physical contact, and one or more other components may be positioned therebetween.

The terms “including” and “comprising”, as used herein, mean the same thing.

The terms “substantially”, “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, they can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. If used herein, the terms “substantially”, “approximately”, “about”, “relatively,” or other such similar terms may also refer to no fluctuations, that is, ±0%.

As used herein, a “flow cell” can include a device optionally having a lid extending over a reaction structure to form a flow channel therebetween that is in communication with a plurality of reaction sites (e.g., nanowells) of the reaction structure, and can optionally include a detection device that detects designated reactions that occur at or proximate to the reaction sites. A flow cell may include a solid-state light detection or “imaging” device, such as a Charge-Coupled Device (CCD) or Complementary Metal-Oxide Semiconductor (CMOS) (light) detection device. For example, the image sensor structure of a sensor system can include an image layer disposed over a base substrate. The image layer may be a dielectric layer, such as SiN and may contain an array of light detectors disposed therein. A light detector as used herein may be, for example, a semiconductor, such as a photodiode, a complementary metal oxide semiconductor (CMOS) material, or both. The light detectors detect light photons of emissive light that is emitted from the fluorescent tags attached to the strands supported in or on the reaction sites, for example, in nanowells. The base substrate may be glass, silicon or other like material. As another specific example, a flow cell can fluidically and electrically couple to a cartridge (having an integrated pump), which can fluidically and/or electrically couple to a bioassay system. A cartridge and/or bioassay system may deliver a reaction solution to reaction sites of a flow cell according to a predetermined protocol (e.g., sequencing-by-synthesis), and perform a plurality of imaging events. For example, a cartridge and/or bioassay system may direct one or more reaction solutions through the flow channel of the flow cell, and thereby along the reaction sites. At least one of the reaction solutions may include four types of nucleotides having the same or different fluorescent labels. In some examples, the nucleotides bind to the reaction sites of the flow cell, such as to corresponding oligonucleotides at the reaction sites. The cartridge and/or bioassay system in these examples then illuminates the reaction sites using an excitation light source (e.g., solid-state light sources, such as light-emitting diodes (LEDs), and lasers). In some examples, the excitation light has a predetermined wavelength or wavelengths, including a range of wavelengths. The fluorescent labels excited by the incident excitation light may provide emission signals (e.g., light of a wavelength or wavelengths that differ from the excitation light and, potentially, each other) that may be detected by the light sensors of the flow cell.

Flow cells described herein perform various biological or chemical processes. More specifically, the flow cells described herein may be used in various processes and systems where it is desired to detect an event, property, quality, or characteristic that is indicative of a designated reaction. For example, flow cells described herein may include or be integrated with light detection devices, sensors, including but not limited to, biosensors, and their components, as well as bioassay systems that operate with sensors, including biosensors.

The flow cells facilitate a plurality of designated reactions that may be detected individually or collectively. The flow cells perform numerous cycles in which the plurality of designated reactions occurs in parallel. For example, the flow cells may be used to sequence a dense array of DNA features through iterative cycles of enzymatic manipulation and light or image detection/acquisition. As such, the flow cells may be in fluidic communication with one or more microfluidic channels that deliver reagents or other reaction components in a reaction solution to a reaction site of the flow cells. The reaction sites may be provided or spaced apart in a predetermined manner, such as in a uniform or repeating pattern. Alternatively, the reaction sites may be randomly distributed. Each of the reaction sites may be associated with one or more light guides and one or more light sensors that detect light from the associated reaction site. In one example, light guides include one or more filters for filtering certain wavelengths of light. The light guides may be, for example, an absorption filter (e.g., an organic absorption filter) such that the filter material absorbs a certain wavelength (or range of wavelengths) and allows at least one predetermined wavelength (or range of wavelengths) to pass therethrough. In some flow cells, the reaction sites may be located in reaction recesses or chambers, which may at least partially compartmentalize the designated reactions therein.

As used herein, a “designated reaction” includes a change in at least one of a chemical, electrical, physical, or optical property (or quality) of a chemical or biological substance of interest, such as an analyte-of-interest. In particular flow cells, a designated reaction is a positive binding event, such as incorporation of a fluorescently labeled biomolecule with an analyte-of-interest, for example. More generally, a designated reaction may be a chemical transformation, chemical change, or chemical interaction. A designated reaction may also be a change in electrical properties. In particular flow cells, a designated reaction includes the incorporation of a fluorescently labeled molecule with an analyte. The analyte may be an oligonucleotide and the fluorescently labeled molecule may be a nucleotide. A designated reaction may be detected when an excitation light is directed toward the oligonucleotide having the labeled nucleotide, and the fluorophore emits a detectable fluorescent signal. In another example of flow cells, the detected fluorescence is a result of chemiluminescence or bioluminescence. A designated reaction may also increase fluorescence (or Förster) resonance energy transfer (FRET), for example, by bringing a donor fluorophore in proximity to an acceptor fluorophore, decrease FRET by separating donor and acceptor fluorophores, increase fluorescence by separating a quencher from a fluorophore, or decrease fluorescence by co-locating a quencher and fluorophore.

As used herein, “electrically coupled” and “optically coupled” refers to a transfer of electrical energy and light waves, respectively, between any combination of a power source, an electrode, a conductive portion of a substrate, a droplet, a conductive trace, wire, waveguide, nanostructures, other circuit segment and the like. The terms electrically coupled and optically coupled may be utilized in connection with direct or indirect connections and may pass through various intermediaries, such as a fluid intermediary, an air gap and the like.

As used herein, a “reaction solution,” “reaction component” or “reactant” includes any substance that may be used to obtain at least one designated reaction. For example, potential reaction components include reagents, enzymes, samples, other biomolecules, and buffer solutions, for example. The reaction components may be delivered to a reaction site in the flow cells disclosed herein in a solution and/or immobilized at a reaction site. The reaction components may interact directly or indirectly with another substance, such as an analyte-of-interest immobilized at a reaction site of the flow cell.

As used herein, the term “reaction site” is a localized region where at least one designated reaction may occur. Reaction sites in the context of the biosensors described herein can also be referred to as nanowells. A reaction site may include support surfaces of a reaction structure or substrate where a substance may be immobilized thereon. For example, a reaction site may include a surface of a reaction structure (which may be positioned in a channel of a flow cell) that has a reaction component thereon, such as a colony of nucleic acids thereon. In some flow cells, the nucleic acids in the colony have the same sequence, being for example, clonal copies of a single stranded or double stranded template. However, in some flow cells a reaction site may contain only a single nucleic acid molecule, for example, in a single stranded or double stranded form.

The terms “active surface” and “active area” are used herein to characterize a surface or area of a reaction structure which operates to support one or more designation reactions. Throughout this disclosure, the terms die and wafer are also used in reference to certain examples herein, as a die can include a sensor and the die is fabricated from a wafer. The words wafer and substrate are also used interchangeably herein.

Examples described herein may be used in various biological or chemical processes and systems for academic or commercial analysis. More specifically, examples described herein may be used in various processes and systems where it is desired to detect an event, property, quality, or characteristic that is indicative of a designated reaction. For instance, examples described herein include cartridges, biosensors, and their components as well as bioassay systems that operate with cartridges and biosensors. In particular examples, the cartridges and biosensors include a flow cell and one or more image sensors that are coupled together in a substantially unitary structure.

The bioassay systems may be configured to perform a plurality of designated reactions that may be detected individually or collectively. The biosensors and bioassay systems may be configured to perform numerous cycles in which the plurality of designated reactions occurs in parallel. For example, the bioassay systems may be used to sequence a dense array of DNA features through iterative cycles of enzymatic manipulation and image acquisition. Alternatively, rather than iterative cycles, the bioassay system can also be used to sequence a dense array of DNA features utilizing continuous observation without stepwise enzymatic action. The cartridges and biosensors may include one or more microfluidic channels that deliver reagents or other reaction components to a well or reaction site. Some examples discussed herein utilize wells and/or nano-wells as reactions sites. However, as used herein, the term “reaction site” is not limited to wells or nano-wells and contemplates various structures on a surface of the examples described herein.

In some examples, the wells or reaction sites are randomly distributed across a substantially planar surface. For example, the wells or reaction sites may have an uneven distribution in which some wells or reaction sites are located closer to each other than other wells or reaction sites. In other examples, the wells or reaction sites are patterned across a substantially planar surface in a predetermined manner. Each of the wells or reaction sites may be associated with one or more image sensors that detect light from the associated reaction site. Yet in other examples, the wells or reaction sites are located in reaction chambers that compartmentalize the designated reactions therein.

In some examples, image sensors may detect light emitted from wells (e.g., nanowells) or reaction sites and the signals indicating photons emitted from the wells or reaction sites and detected by the individual image sensors may be referred to as those sensors' illumination values. These illumination values may be combined into an image indicating photons as detected from the wells or reaction sites. Such an image may be referred to as a raw image. Similarly, when an image is composed of values which have been processed, such as to computationally correct for crosstalk, rather than being composed of the values directly detected by individual image sensors, that image may be referred to as a sharpened image.

In some examples, image sensors (e.g., photodiodes) are associated with corresponding wells or reaction sites. An image sensor that is associated with a reaction site is configured to detect light emissions from the associated reaction site when a designated reaction has occurred at the associated reaction site. In some cases, a plurality of image sensors (e.g., several pixels of a camera device) may be associated with a single reaction site. In other cases, a single image sensor (e.g., a single pixel) may be associated with a single reaction site or with a group of wells or reaction sites. The image sensor, the reaction site, and other features of the biosensor may be configured so that at least some of the light is directly detected by the image sensor without being reflected.

Depending on the context, the term “image sensor” is utilized interchangeably herein to refer to both an array of individual pixels/photodiodes and/or an individual light sensor or pixel (which the array comprises). In the context of the examples described herein, an image sensor, which is an array, generates a signal. The sensors discussed in the examples herein, which can include image sensors can include front side illuminated sensors (FSIs) and back-side illuminated sensors (BSIs).

As used herein, the term “adjacent” when used with respect to two wells or reaction sites means no other reaction site is located between the two wells or reaction sites. The term “adjacent” may have a similar meaning when used with respect to adjacent detection paths and adjacent image sensors (e.g., adjacent image sensors have no other image sensor therebetween). In some cases, a reaction site may not be adjacent to another reaction site; but may still be within an immediate vicinity of the other reaction site. A first reaction site may be in the immediate vicinity of a second reaction site when fluorescent emission signals from the first reaction site are detected by the image sensor associated with the second reaction site. More specifically, a first reaction site may be in the immediate vicinity of a second reaction site when the image sensor associated with the second reaction site detects, for example, crosstalk from the first reaction site. Adjacent wells or reaction sites may be contiguous, such that they abut each other, or the adjacent sites may be non-contiguous, having an intervening or interstitial space between.

As used herein, the term “crosstalk” refers to any phenomenon by which a signal transmitted on one circuit or channel of a transmission system creates an undesired effect in another circuit or channel. Crosstalk is usually caused by undesired capacitive, inductive, or conductive coupling from one circuit or channel to another. Crosstalk can be a significant issue in structured cabling, audio electronics, integrated circuit design, wireless communication, and other communications systems. In the context of certain of the examples herein, crosstalk includes a proportion of optical signals from a given reaction site reaching light sensors or pixels that do not form a sensing pair with the reaction site. In examples where each image sensor represents a single pixel, crosstalk may be understood to mean the proportion of optical signals reaching all pixels other than the center pixel. Attenuation, or signal loss, can result from crosstalk. Additionally, crosstalk increases noise in pixels within an immediate vicinity of a reaction center.

As used herein, the term loss-induced crosstalk reduction or “LICR” refers to a tailored absorption of light that might otherwise result in crosstalk. While certain LICR features may not eliminate crosstalk, as discussed herein, they can reduce it to a degree where any remaining crosstalk may be computationally corrected through conventional image processing techniques (where such image processing techniques, alone, may be insufficient in the absence of the LICR features described herein). Based on LICR, a signal at neighboring pixels is substantially lower than a signal at paired pixels.

As used herein the term “emission filter” refers to a filter that suitably prevents/blocks transmission of excitation wavelengths while suitably allowing transmission of emission wavelengths. For example, an emission filter can be a high quality optical-glass filter commonly used in fluorescence microscopy and spectroscopic applications for selection of the excitation wavelength of light from the light source. An excitation wavelength is a wavelength in the excitation spectrum, a range of light wavelengths that add energy to a fluorochrome, causing it to emit wavelengths of light (e.g., the emission spectrum).

The term chemical vapor deposition (CVD) refers to a vacuum deposition method used to produce high quality, and high-performance, solid materials, including, in some of the examples herein, films. In some examples, a substrate (e.g., a silicon wafer) wafer (substrate) is exposed to one or more volatile precursors, which react and/or decompose on the substrate surface to produce a desired deposit. As discussed herein, plasma-enhanced chemical vapor deposition (PECVD) is a chemical vapor deposition process used to deposit thin films from a gas state (vapor) to a solid state on a substrate. In the context of the examples herein, CVD and/or specifically PECVD is utilized to deposit an oxide layer with a low index of refraction (referred to also as a low index oxide layer, e.g., SiO (silicon monoxide)) on certain of the apparatuses discussed. This description also includes references to high index oxide materials, which refer to materials with a high index of refraction, including but not limited to SiN (silicon nitride).

The term chemical mechanical polishing or planarization (CMP) is a process (both polishing and planarization being options under the umbrella term) applied to selectively remove materials for topography planarization and device structure formation. CMP uses chemical oxidation and mechanical abrasion to remove material and achieve planarity. In some examples, CMP includes using a chemical reaction and mechanical abrasion with slurries containing unique chemical formulations and large numbers of abrasive particles. During polishing, chemical reaction products and mechanical wear debris are generated. Slurry particles and polishing byproducts are pressed onto wafer surface. During wafer transferring from polisher to cleaner, contaminants are adhered onto wafer surface. This process can include a cleanup of the surface that is polished and/or planarized to remove particles including organic residues. Certain of the workflows disclosed herein incorporate a CMP aspect to planarize surfaces. CMP can be utilized in the examples herein, for example, after depositions into high aspect ratio topography, which may impact the topography of the deposited top film (i.e., layer). However, even when incorporated into the examples herein, in some circumstances, this aspect can be omitted.

Various examples herein include a layer of germanium. Some examples reference silicon germanium SiGe, specifically. This example is provided for illustrative purposes and the germanium layers referenced, in various examples, can comprise silicon germanium, Si(x)Ge(1-x) or SiₓGe₁₋ₓ).

Reference is made below to the drawings, which are not drawn to scale for ease of understanding, wherein the same reference numbers are used throughout different figures, in some cases, to designate the same or similar components. The following detailed description of certain examples will be better understood when read in conjunction with the appended drawings. To the extent that the figures illustrate diagrams of the functional blocks of various examples, the functional blocks are not necessarily indicative of the division between hardware components. Thus, for example, one or more of the functional blocks (e.g., processors or memories) may be implemented in a single piece of hardware (e.g., a general-purpose signal processor or random-access memory, hard disk, or the like). Similarly, the programs may be standalone programs, may be incorporated as subroutines in an operating system, may be functions in an installed software package, and the like. The various examples are not limited to the arrangements and instrumentality shown in the drawings.

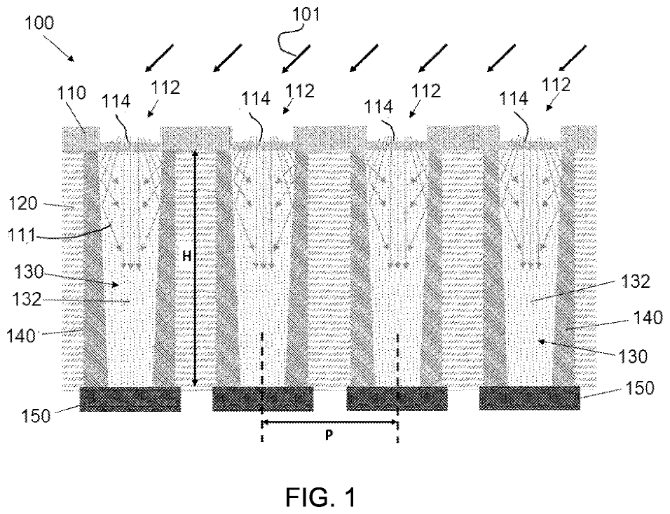

It is desirable to reduce crosstalk in sensors, including in biosensors utilized in flow cells as crosstalk adversely affects performance. A traditional way in which crosstalk can be reduced in an apparatus with an image sensor, such as a flow cell, is by physically constraining transmission of light by embedding various light guides in the sensor, including but not limited to curtain structures, light pipes, and/or optical waveguides and/or microlenses. These structures direct light emitted from a corresponding reaction site directly downwardly toward an image sensor that forms a sensing pair with the reaction site. As will be described herein in reference to FIG. 1, these structures reduce crosstalk by physically blocking light to provide tailored absorption of light that might otherwise result in crosstalk. As is the case with the LICR layer, discussed in later examples, the physical structures in FIG. 1 reduce crosstalk.

Because of the manufacturing complexities and structural limitations associated with sensor devices that include structural elements (e.g., curtain structures, light pipes, and/or optical waveguides) to reduce crosstalk, it is desirable to provide a version of a biosensor that suitably prevents or reduces the occurrence of optical crosstalk, without presenting the manufacturing complexity and expense associated with these structures and without constraining certain parameters of the sensor, including pitch distance, which will be discussed herein. Rather than integrate what are sometimes complex structures into sensing devices to reduce crosstalk, such as the aforementioned curtain structures, light pipes, and/or optical waveguides, which increase the cost and complexity of the sensor devices, examples of sensor devices, also referred to herein as biosensors, described herein instead include at least one layer of germanium that provides LICR and/or an emission filter. LICR features, such as the germanium layer, integrated into biosensors described herein do not completely eliminate crosstalk, but, rather, provide tailored absorption of light that might otherwise result in crosstalk. LICR examples suitably prevent or reduce the occurrence of optical crosstalk, without presenting the manufacturing complexity and expense associated with the structural elements (e.g., curtain structures, light pipes, and/or optical waveguides) and without constraining a reduction of pitch distance in a biosensor. Utilizing layers to reduce crosstalk, rather than structures, such as that in FIG. 1, provide blankets that are uniform in the x-y plane.

As discussed on FIG. 1, structural cross talk reduction elements (e.g., curtain structures, light pipes, and/or optical waveguides) necessarily are of given heights to function (i.e., reduce or eliminate crosstalk), thus ascribing specific pitch minimums to biosensors, requirements avoided by eliminating these structures.

Described herein are structures of examples of sensor devices (e.g., biosensors) which include this at least one germanium layer for LICR and as an emission filter, methods of using these sensor devices, and methods of manufacturing these sensor devices. Manufacturing examples herein include both biosensors that include from an off-the-shelf sensor components, such as an off-the shelf-CMOS, as well as methods that involve forming a custom sensor or CMOS. As noted above, utilizing a sensor device with a germanium layer for LICR instead of a complex structure (e.g., curtain structures, light pipes, and/or optical waveguides) reduces development cost and turn-around time of sensor-based sequencers, including, but not limited to, CMOS-based sequencers. The methods of manufacture utilized to manufacture sensors with curtain structures, light pipes, and/or optical waveguides, involve a high level of customization, increase cost, and, from a performance perspective, add difficulty to increasing reaction site density, when compared with method of manufacture of biosensors with the aforementioned germanium layer.

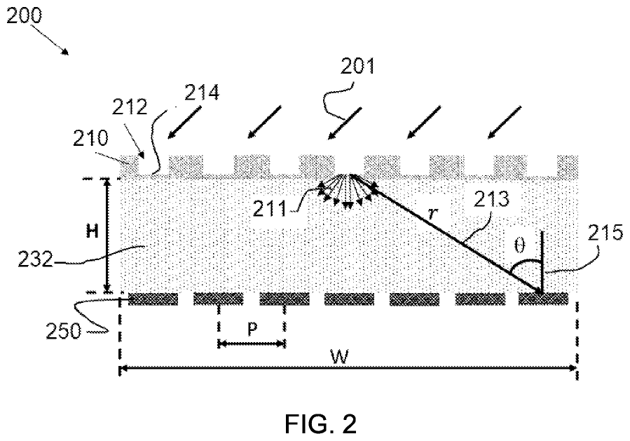

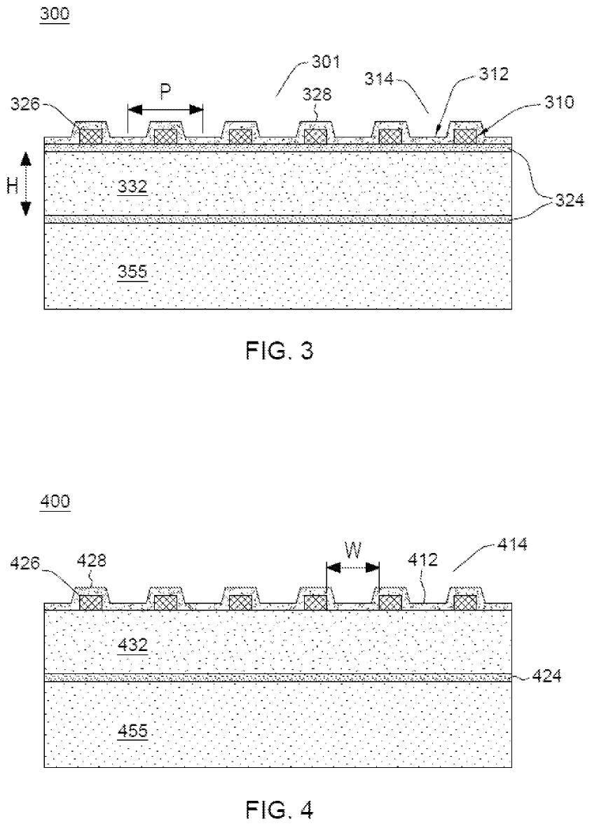

To contrast existing biosensors that utilize structural elements spanning the height of the biosensors for crosstalk reduction and the examples herein which utilize a layer of germanium for LICR and as an emission filter, FIGS. 1-3 illustrate various biosensor configurations. FIG. 1 illustrate an example of a biosensor 100 that includes a complex structure (e.g., curtain structures, light pipes, and/or optical waveguides) to reduce crosstalk. FIG. 2 illustrates a configuration of a biosensor 200 that eliminates the complex structure utilized to reduce cross talk in the biosensor 100 of FIG. 1 and introduces, generally, using a lossy material as both an emission filter and as a medium to reduce crosstalk. FIGS. 3-5 illustrate examples of a biosensor 300 that include germanium as a material utilized for LICR and as an emission filter.

FIG. 1 is an example of a sensor device where cross talk reduction is handled by a curtain structure. FIG. 1 shows a biosensor 100 that includes a flow channel floor 110 defining a plurality of wells 112, with each well 112 providing a reaction site 114. A base 120 underneath the floor 110 defines a plurality of light guides 130, with each light guide 130 being positioned under a corresponding reaction site 114. Each light guide 130 contains a filter material 132. Each light guide 130 also has a tapered profile in this example, such that the upper region of light guide 130 is wider than the lower region of light guide 130, with the width linearly narrowing from the upper region to the lower region.

As biosensor 100 is exposed to excitation light 101 (e.g., as generated by one or more light sources), the excitation light 101 causes fluorophores at reaction sites 114 to emit light 111. The filter material 132 filters out the excitation light 101 without filtering out the emitted light 111. In scenarios where nucleic acids are at reaction sites 114, the emitted light 111 may indicate the composition of such nucleic acids. An image sensor 150 is positioned under each light guide 130 and is configured to receive the light 111 emitted from the corresponding reaction site 114 via the corresponding light guide 130. Thus, each image sensor 150 forms a “sensing pair” with the reaction site 114 that is directly aligned with (e.g., positioned directly above) the image sensor 150. In versions where each image sensor 150 represents a single pixel, the image sensor 150 forming a sensing pair with a reaction site 114 may be referred to as the “center pixel” associated with that reaction site 114; while the image sensors 150 adjacent to the center pixel may be referred to as “neighbor pixels.” Similarly, an image sensor 150 that does not form a sensing pair with a given reaction site 114 may be referred to as a “neighbor sensor” with respect to that reaction site 114.

In some other examples, a single image sensor 150 may receive photons through more than one light guide 130 and/or from more than one reaction site 114. In such versions, the particular region of the single image sensor 150 that is directly aligned with (e.g., positioned directly under) a reaction site 114 may be said to form a “sensing pair” with that reaction site 114.

As shown in FIG. 1, biosensor 100 provides a height distance (H) between each image sensor 150 and the underside of the floor 110 in the region underneath the reaction site 114 forming a sensing pair with that image sensor 150. In this example, this height distance (H) represents the thickness of base 120. By way of example only, this height distance (H) may range from approximately 2 micrometers to approximately 4 micrometers; or may be approximately 3.5 micrometers. Alternatively, biosensor 100 may provide any other suitable height distance (H). As also shown in FIG. 1, biosensor 100 provides a pitch distance (P) that is defined between a central axis of an image sensor 150 and each adjacent image sensor 100. This pitch distance (P) also represents the distance between a central axis of a well 112 and each adjacent well 112. By way of example only, this pitch distance (P) may range from approximately 0.7 micrometers to approximately 2.0 micrometers; or may be approximately 1 micrometer. Alternatively, biosensor 100 may provide any other suitable pitch distance (P).

The biosensor 100 depicted in FIG. 1 includes a plurality of shields or curtains 140. Each curtain 140 surrounds a corresponding light guide 130 and extends the full vertical height of base 120, such that each curtain 140 extends from a corresponding image sensor 150 to floor 110. Curtains 140 thus define interruptions along the width of base 120. Curtains 140 also fully contain corresponding volumes of filter material 132, such that no portions of filter material 132 span across the full width of base 120. Curtains 140 of this example are formed of or otherwise include an opaque material such as metal, though curtains 140 may alternatively be formed of or otherwise include other materials or combinations of materials. Curtains 140 are configured to suitably prevent light 111 emitted at one reaction site 114 from reaching an image sensor 150 that is positioned directly under another reaction site 114. In other words, curtains 140 prevent or suitably reduce the amount of light 111 emitted at a reaction site 114 from reaching image sensors 150 that do not form a sensing pair with that reaction site 114. These curtains 140 thus define light pipes or optical waveguides, ensuring that most of all of light 111 emitted at a given reaction site 114 is at most only received by the image sensor 150 forming a sensing pair with that reaction site 114. In doing so, curtains 140 prevent or suitably reduce the occurrence of optical crosstalk within biosensor 100. This crosstalk includes the proportion of optical signals from a given reaction site 114 reaching image sensors 150 that do not form a sensing pair with the reaction site. In versions where each image sensor 150 represents a single pixel, crosstalk may be understood to mean the proportion of optical signals reaching all pixels other than the center pixel.

The integration of curtains 140 into a biosensor 100 may effectively prevent optical crosstalk within the biosensor 100 by suitably preventing light 111 emitted at a reaction site 114 from reaching an image sensor 150 that does not form a sensing pair with the reaction site 114. However, as noted above generally and demonstrated in this non-limiting example, including curtains 140 in a biosensor 100 may tend to add complexity and expense to the process of manufacturing biosensor 100, especially with curtains 140 extending through the full height distance (H) of biosensor 100. Such complexity and expense may be due, at least in part, to curtains 140 having sub-micron feature sizes (in the x-y plane) and several-micron thickness (in the z direction). Such complexity and expense may also be due, at least in part, to filter material 460 being applied separately within each individual light guide 462.

In addition, it may be desirable to minimize the pitch distance (P) in a biosensor 100 in order to maximize the total number of reaction sites 114 in the biosensor 100 (e.g., to maximize the density of reaction sites 114 in biosensor 100); and the presence of curtains 140 in a biosensor 100 may constrain the reduction of pitch distance (P) in the biosensor 100 since curtains 140 occupy physical space in the biosensor. Thus, it is possible to reduce the pitch distance (P) in the biosensor 100 if curtains 140 are eliminated.

FIG. 2 shows an example of a biosensor 200 that lacks structural elements that span its height, such as the curtains 140 in the biosensor 100 of FIG. 1, to manage crosstalk. Biosensor 200 of this example includes a flow channel floor 210 defining a plurality of wells 212, with each well 212 providing a reaction site 214. A layer 232 of filter material is positioned under flow channel floor 210. A plurality of image sensors 250 are positioned under the layer 232 of filter material. Each image sensor 250 is vertically centered under a corresponding well 212 and reaction site 214, such that each sensor 250 forms sensing pair with a corresponding reaction site 214 (e.g., nanowells). In this example, the layer 232 of filter material in biosensor 200 effectively forms a structural equivalent of base 120 in biosensor 100. The layer 232 of filter material spans the full height distance (H) and width distance (W) of biosensor 200. In other words, the layer 232 of filter material spans uninterrupted or contiguously under the wells 212 and reaction sites 214.

As biosensor 200 is exposed to excitation light 201 (e.g., as generated by one or more light sources), the excitation light 201 causes fluorophores at reaction sites 214 to emit light 211. In scenarios where nucleic acids are at reaction sites 214, the emitted light 211 may indicate the composition of such nucleic acids. Image sensors 250 receive the light 211 emitted from the reaction sites 214 via the layer 232 of filter material. The filter material of layer 232 filters out the excitation light 201 without filtering out the emitted light 211. As will be discussed herein, including in FIG. 3, the filter material can include germanium. As shown, the layer 232 of filter material prevents substantial transmission of substantially all wavelengths of excitation light 201 while permitting transmission of a proportion of some wavelengths of emitted light 211. In some examples, the transmitted proportion is approximately 0.01 to approximately 10%.

Since biosensor 200 of the example shown in FIG. 2 lacks light-blocking features like curtains 140, and since the filter material of layer 232 is not configured to filter emitted light 211, emitted light 211 from any given reaction site 214 may reach one or more image sensors 250 that do not form a sensing pair with the reaction site 214. In other words, emitted light 211 from any given reaction site 214 may reach one or more image sensors 250 that are not directly underneath the reaction site 214. Thus, biosensor 200 generates crosstalk as emitted light 211 from a given reaction site 214 propagates through layer 232 of filter material at non-vertical angles to reach various image sensors 250 that do not form a sensing pair with the reaction site 214. In other words, biosensor 200 generates crosstalk as emitted light 211 from a given reaction site 214. The emitted light 211 propagates through layer 232 of filter material at non-vertical angles to reach image sensors 250 that are not directly below the reaction site 214. FIG. 2 shows such crosstalk occurring along an optical path having a length (r) and defining an angle (θ) with an axis 215 that is normal to the image sensor 250 receiving the light 211.

The distribution of an optical signal from light 211 emitted from a single reaction site 214 over the image sensors 250 of biosensor 200 may be defined as a point-spread function (PSF). The PSF may thus represent the degree of crosstalk occurring within biosensor 200. The PSF may depend on the height-to-pitch ratio (HIP), as shown below in Equation I:

$$PSF ( r , θ ) ∝ \frac{cos ( θ )}{{r}^{2}} = \frac{H}{{r}^{3}} \qquad ( I )$$

where “PSF” is the point spread function;
“r” is the length of the optical path between the reaction site 214 from which the light 211 is being emitted;
“θ” is the angle defined between the optical path of “r” and an axis 215 that is normal to the image sensor 250 receiving the emitted light 211; and
“H” is the height of the layer 232 of filter material.

FIG. 3 illustrates a biosensor 300, which can be utilized as a submicron pitch image sensor, where a filter layer 332 is comprised of germanium (e.g., germanium, silicon germanium, Si(x)Ge(1-x)). The layer 332 of germanium spans the full height distance (H) and width distance (W) of biosensor 300. The biosensor 300 of this example includes a flow channel floor 310 defining a plurality of wells 312 (e.g., nanowells), with each well 312 providing a reaction site 314. A layer 332 of filter material, including germanium (e.g., silicon germanium, Si(x)Ge(1-x)) is positioned under flow channel floor 310. In other words, the layer 332 of germanium spans uninterrupted or contiguously under the plurality of wells 312. In some examples, this layer, which includes germanium, has a height (H) of approximately 200 nanometers to approximately 500 nanometers.

In this non-limiting example, the reaction sites 314 and wells 312 are comprised of multiple oxide layers and/or of another dielectric material (e.g., NiO, SiO2, tantalum pentoxide, Si3N4, etc.) 328 and a sensor (e.g., CMOS) compatible metal (e.g., aluminum, tantalum, etc.) 326. By way of example and not to impose or suggest any limitations, the compatible metal 326 can be approximately 200 to approximately 500 nm.

An image sensor 355 (e.g., a backside image sensor with deep trenches) is positioned under the layer 332 that includes germanium. This layer 332 serves as both a filter layer and a LICR layer. In this example, between the layer 332 and the sensor 355 is one or more layers of isolation oxide and/or of another dielectric material (e.g., SiO2, NiO, Si3N4, etc.) 324. One or more layers of isolation oxide and/or of another dielectric material (e.g., SiO2, NiO, Si3N4, etc.) 324 are also situated between the layer 332 and the multiple oxide layers and/or layers of another dielectric material (e.g., NiO, SiO2, Si3N4, tantalum pentoxide, etc.) 328 and the sensor (e.g., CMOS) compatible metal (e.g., aluminum, tantalum, etc.) 326, below the base of the latter. Although not pictured in FIG. 3, in some examples, as previously illustrated in FIG. 2, the sensor 355 can comprise one or more sensors which are vertically centered under a corresponding well 312 and reaction site 314, such that each sensor forms sensing pair with a corresponding reaction site 314.

As biosensor 300 is exposed to excitation light 301 (e.g., as generated by one or more light sources), the excitation light 301 causes fluorophores at reaction sites 314 to emit light 311. In scenarios where nucleic acids are at reaction sites 314, the emitted light 311 may indicate the composition of such nucleic acids. The image sensor 355 receives light emitted from the reaction sites 314 via the layer 332 of filter material. The filter material of layer 332 filters out the excitation light 301 without substantially filtering out the emitted light. The layer 332 of filter material suitably prevents transmission of substantially all or all wavelengths of excitation light 301 while permitting transmission of all or substantially all wavelengths of emitted light. As is the case with the biosensor of FIG. 3, the distribution of an optical signal from light emitted from a single reaction site 314 over the image sensor 355 of biosensor 300 may be defined as a point-spread function (PSF).

As discussed above, an advantage of eliminating complex structures for crosstalk reduction or elimination, such as the curtains 140 of FIG. 1 (which define light pipes or optical waveguides), is an ability to more easily manage and/or customize pitch distance (P). In the biosensor 300 of FIG. 3, the pitch distance (P) represents the distance between a central axis of a well 312 and each adjacent well 312. While pitch distances for a biosensor 100 with the complex structural elements illustrated in FIG. 1 may range from approximately 0.7 micrometers to approximately 2.0 micrometers, including being approximately 1 micrometer, in the absence of these structural constraints, by way of example only, the pitch distance (P) of a biosensor 300 such as that in FIG. 3 may range from approximately 0.55 micrometers to approximately 0.7 micrometers.

FIG. 4 is an example of a biosensor 400 that includes a filter layer 432 that is comprised of germanium (e.g., silicon germanium, Si(x)Ge(1-x)), but FIG. 4 is a cross-section of the biosensor 400. Visible from this perspective are one or more isolation oxide and/or another dielectric material (e.g., SiO2, NiO, Si3N4, etc.) layers 424 as well as reaction sites 414 and wells 412 that are comprised of multiple oxide layers and/or layers of another dielectric material (e.g., NiO, SiO2, Si3N4, tantalum pentoxide, etc.) 428 and a sensor (e.g., CMOS) compatible metal (e.g., aluminum, tantalum, etc.) 426. As described in reference to FIG. 3, in this example, of a biosensor 400, the image sensor 455 receives light emitted from the reaction sites 314 via the layer 332 of filter material. In a non-limiting example, a width (w) of the wells 412 as seen in this cross-sectional view is approximately 0.4 micrometers.

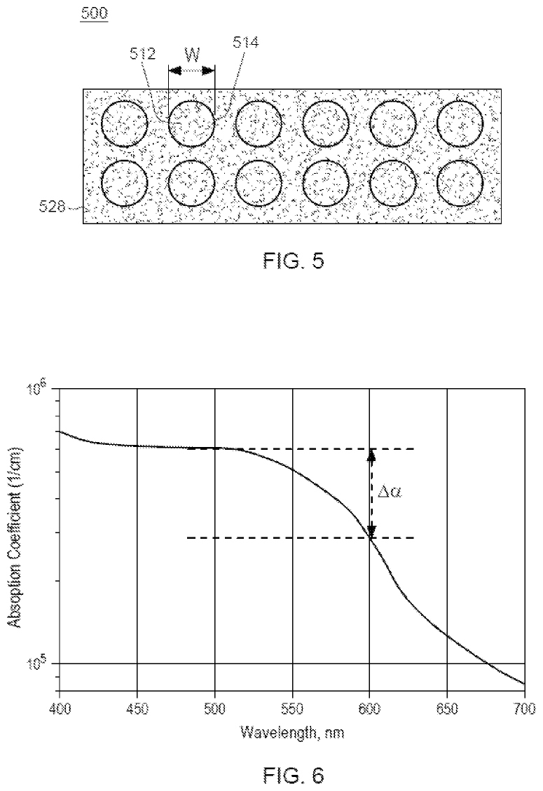

As noted above, FIG. 5 is also example of a biosensor 500 that includes a filter layer that is comprised of germanium (e.g., silicon germanium, Si(x)Ge(1-x)) but given that this figure is shown as a top view, this layer is not visible from this perspective. However, visible from the view of FIG. 5 are reaction sites 514 and wells 512 and the multiple oxide layers and/or layers of another dielectric material (e.g., NiO, SiO2, Si3N4, tantalum pentoxide, etc.) 528. Additionally, a width (w) of the wells 412 visible in FIG. 4 is also visible as a width (w) of the wells 512 in FIG. 5. In a non-limiting example, a width (w) of the wells 512 as seen in this cross-sectional view is approximately 0.4 micrometers.

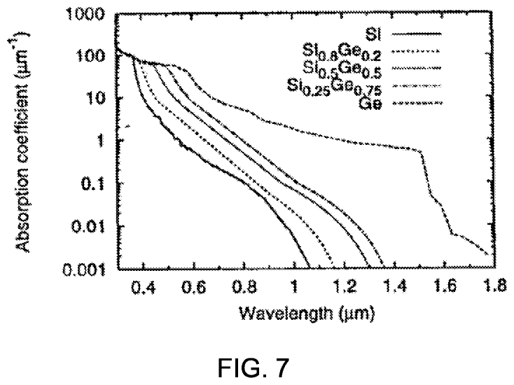

Germanium and composite materials that include germanium can be utilized effectively in biosensors for LICR and as an emission filter at least because properties of germanium and of silicon germanium, including the absorption coefficient of these materials are conducive to this use. FIGS. 6-7 demonstrate aspects of the absorption of germanium and certain materials that include germanium which enable germanium to reduce crosstalk as well as provide an emission filter when integrated into a biosensor.

Turning first to FIG. 6, this plot illustrates how germanium has a very high absorption difference between red and green, as represented by Equation II below,

$$\frac{Q {E}_{red}}{Q {E}_{green}} = {e}^{Δ α H} \qquad ( II )$$

In Equation II, QE is an emission or excitation wavelength. Thus. QE_{red} is the emission wavelength of red while QE_{green} is the excitation wavelength of green. H represents the thickness of the germanium and Δa=a_{red}−a_{green}=300,000 1/cm.

Thus, H, the thickness of the germanium, can be represented by Equation III.

$$H = \frac{1}{Δα} ln ( \frac{Q {E}_{red}}{Q {E}_{green}} ) \qquad ( III )$$

Thus, when the absorption of red and green is compared, it is greater than 105, as illustrated in Equation IV below.

$$\frac{Q {E}_{red}}{Q {E}_{green}} > {10}^{5} \qquad ( IV )$$

When the calculations in this non-limiting example are complete the thickness, H, is found to be greater than 360 nanometers, as illustrated in Result V below.

H>360 nm (V)

The thickness of the germanium layer utilized in various examples of the biosensors described herein is selected based on a relationship between excitation wavelength QE (e.g., green and blue) and emission wavelength QE (e.g., red). To optimize functionality is some examples of the biosensors described herein, the germanium layer would increase red QE and reduce blue and green QE. For the purpose of ease in fabrication combined with performance gains, it is desirable to utilize a layer of the lowest thickness that gives a high enough

$$\frac{Q {E}_{red}}{Q {E}_{green}}$$

ratio to act as an effective emission filter. Higher thickness improves

$$\frac{Q {E}_{red}}{Q {E}_{green}}$$

but reduces the absolute amount of QE_{red}, which can adversely affect the operation of the biosensor. Thus, an optimum value for the thickness would cause the layer to operate as a filter that performs enough excitation rejection (the ratio) and receives enough of the signal (red QE). In some examples, this optimum value for thickness is expressed as

$$\frac{Q {E}_{red}}{Q {E}_{green}} = ∼ 1 \times {10}^{∧} 5 .$$

Now turning to FIG. 7, a plot which illustrates the absorption coefficients of various compounds of silicon germanium (e.g., Si(x)Ge(1-x)). These different compounds can be utilized in the biosensors 300, 400, and 500, in layers that provide LICR and act as an emission filter. AS illustrated in FIG. 7, x=0.8 has the highest ratio of absorption at 0.6 um and 0.5 um wavelength. Thus, SiGe is demonstrated potentially to have better mechanical properties compared to pure Ge when utilized in an LICR and filter layer of a biosensor in certain examples of the biosensors discussed herein.

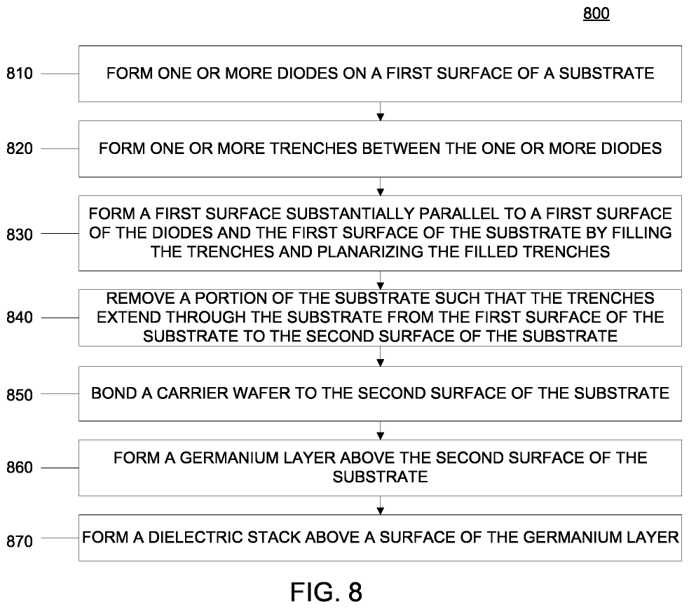

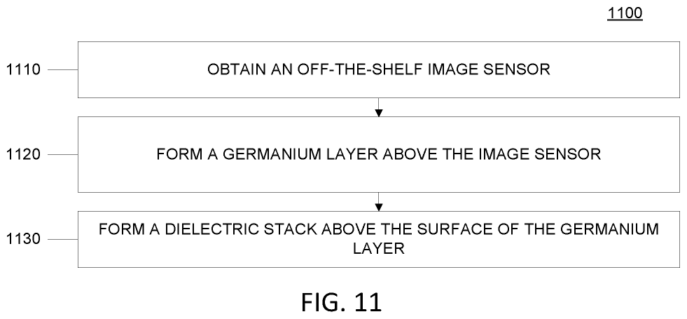

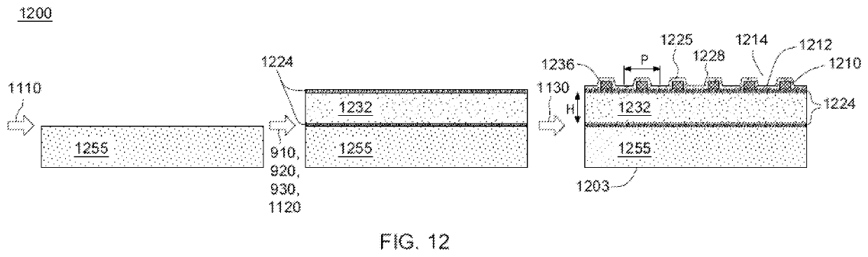

As discussed above, biosensors that include germanium (e.g., silicon germanium, Si(x)Ge(1-x)) can be fabricated utilizing various methods discussed herein. However, certain of the methods can include fabricating an image sensor (e.g., CMOS) as a custom part of the biosensor while other methods can utilize off-the-shelf image sensors and deposit a germanium layer. Non-limiting examples of both types of fabrication/manufacturing processes are illustrated herein. FIGS. 8-10 illustrates the fabrication of a non-limiting example of a biosensor that includes fabrication of an image sensor for utilization in the biosensor, while FIGS. 11-12 illustrates a fabrication process for a biosensor with a germanium layer for LICR and filtering where a commercially available non-custom image sensor (e.g., CMOS) is integrated into the resultant biosensor.

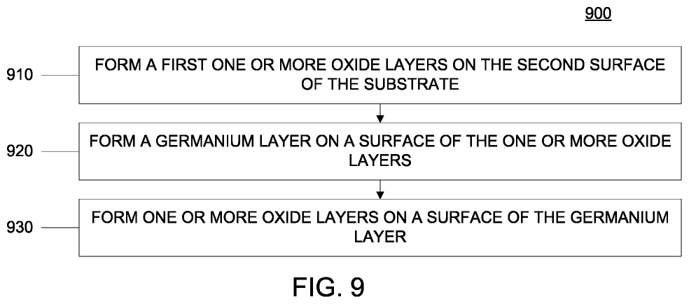

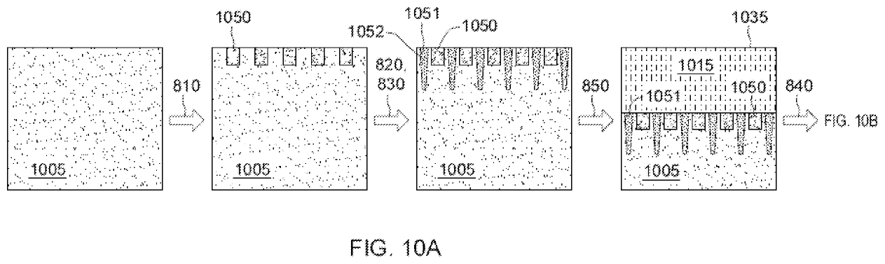

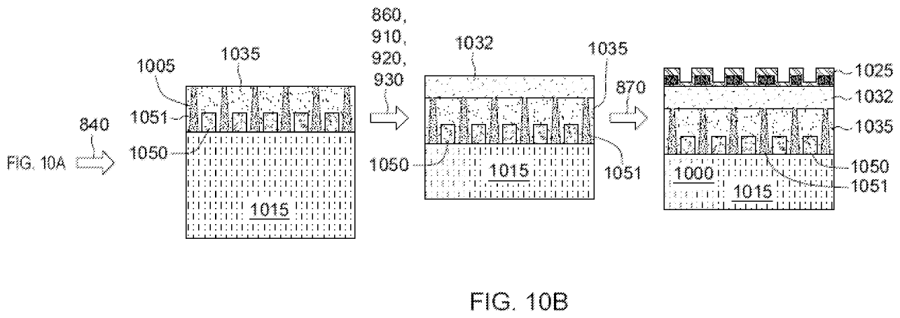

FIG. 8 is a workflow 800 that illustrates an example of a fabrication process for a biosensor that includes a germanium layer for both LICR and filtering. As illustrated in FIG. 8 as well as in FIGS. 9-10, the method includes: 1) forming diodes on a substrate (e.g., a silicon wafer); 2) forming trenches in the substrate (e.g., etching the trenches, filling the trenches, and planarization the resultant surface); 3) bonding a carrier wafer to the substrate; 4) thinning the carrier wafer; 5) depositing oxide and germanium on silicon/substrate combination; and 6) forming nanowells.

Referring to FIG. 8, as illustrated in the workflow, the method includes forming one or more diodes on a first surface of a substrate (810). The substrate may be comprised of silicon and can be understood as a silicon wafer. In this example, the substrate has two surfaces that are parallel to each other, that for clarity in this workflow 800 are referred to as the first surface and a second surface of the substrate.

The workflow 800 also includes forming one or more trenches between the one or more diodes (820). These trenches extend toward the second surface of the substrate from the first surface of the substrate. Various methods can be utilized to form these trenches, including but not limited to etching the one or more trenches in the substrate.

The workflow 800 includes forming a first surface substantially parallel to a first surface of the diodes and the first surface of the substrate by filling the trenches and planarizing the filled trenches (830). In some examples, the trenches are filled with one or more oxide layers and/or one or more dielectric layers (which do not include oxide). As noted above, some methods of forming or manufacturing examples of the biosensors described herein, which include at least one layer with germanium for LICR and filtering, start with a pre-existing sensor while others include forming the sensor.

The workflow 800 can include removing a portion of the substrate such that the trenches extend through the substrate from the first surface of the substrate to the second surface of the substrate (840). The workflow 800 includes bonding a carrier wafer to the second surface of the substrate (850). In some examples, the carrier wafer is bonded to the surface of the substrate before a portion of the substrate is removed and in others, the portion of the substrate is removed before bonding the carrier wafer to this sensor structure. The workflow 800 of FIG. 8 includes forming a sensor and this sensor includes the substrate, the diodes, the carrier wafter, and the filled trenches. This sensor can be, for example, a CMOS.

The workflow 800 includes forming a germanium layer above the second surface of the substrate (860). The layer can be formed utilizing various techniques. For example, one can deposit the germanium layer on the surface of the substrate. One non-limiting technique that can be utilized to deposit this germanium is Plasma-enhanced chemical vapor deposition (PECVD). PECVD is a chemical vapor deposition process used to deposit thin films from a gas state (vapor) to a solid state on a substrate. As part of this process plasma is created, for example, by radio frequency (RF) (alternating current (AC)) frequency or direct current (DC) discharge between two electrodes, the space between which is filled with the reacting gases. In some examples, one deposits the germanium layer at a low temperature (e.g., ˜200-˜300 C) as part of the PECVD process. Other techniques that can be utilized to deposit the germanium layer (a layer that includes germanium but can also include silicon, as explained above) include, but are not limited to, sputter, e-beam evaporation, crystalline growth and etching, and radical activation bonding in vacuum. In some examples, when crystalline growth and etching is utilized, this includes either transfer wafer bonding or direct wafer bonding. Table 1 below lists various of the deposition techniques discussed earlier that can be utilized in various examples to deposit a germanium layer above the second surface of the substrate. In each case, the technique, the material, and a non-limiting example of one or more approximate temperatures is provided.

TABLE 1

| Deposition Techniques | Material | Temperature |
|---|---|---|
| PECVD | a-Ge, poly-Ge | 250-400 C. |
| Sputter | a-Ge, poly-Ge | 100-450 C. |
| E-beam evaporation | a-Ge, poly-Ge | 200-400 C. |
| Transfer/Direct wafer | c-Ge | 200-300 C.-direct |
| bonding | | wafer bonding |
| Molecular-beam | c-Ge | 370-600 C. |
| epitaxy (MBE) | | |

FIG. 9 is a workflow 900 for forming the germanium layer in a biosensor. This later provides LICR and filtering. This workflow 900 commences by forming a first one or more oxide layers and/or one or more dielectric layers on the second surface of the substrate (910). Upon forming the oxide layer, an individual or machine forms a germanium layer on a surface of the one or more oxide layers and/or one or more dielectric layers (920). In this workflow, an individual or machine then forms one or more oxide layers and/or one or more dielectric layers on a surface of the germanium layer (930). As illustrated in FIG. 3, these oxide layers can be understood as one or more layers of isolation oxide and/or another dielectric material (e.g., SiO2, NiO, Si3N4, etc.) 324 and are situated on either side of the filter layer 332.

Returning to FIG. 8, the workflow 800 also includes forming a dielectric stack above a surface of the germanium layer (870). This dielectric stack can include one or more nanowells (e.g., reaction sites and wells). FIGS. 3-5 include examples of at least portions of dielectric stacks that can be formed in examples of this workflow 800. The dielectric stack can be formed from multiple oxide layers and/or layers of another dielectric material (e.g., NiO, SiO2, Si3N4, tantalum pentoxide, etc.) and a sensor (e.g., CMOS) compatible metal (e.g., aluminum, tantalum, etc.). By way of example and not to impose or suggest any limitations, the compatible metal can be approximately 200 to approximately 500 nm.

FIGS. 10A-10B, referred to collectively as FIG. 10, illustrate aspects of the workflows 800, 900 of FIGS. 8 and 9 but includes visual representations for the biosensor 1000 in various states during the performance of the workflows 800, 900. To illustrate these aspect, certain of the labels from FIGS. 8 and 9 are provided in FIG. 10.

Referring to FIG. 10A, one or more diodes 1050 are formed on a first surface of a substrate 1005 (810). The diodes may be one or more image sensors (e.g., FIG. 2, image sensors 250). The substrate may be comprised of silicon and can be understood as a silicon wafer. Trenches 1052 are formed between the diodes 1050 (820). Various methods can be utilized to form these trenches, including but not limited to etching the one or more trenches in the substrate. The trenches are filled with one or more oxide layers and/or one or more dielectric layers to form filled trenches 1051. After the trenches 1052 are filled, the resultant surface is planarized to form a surface that is substantially parallel to the surface of the substrate 1005 that includes the diodes 1050 (830). Thus, a sensor portion 1035 (e.g., CMOS) of the biosensor 1000 is now complete.

As mentioned when FIG. 8 was discussed, at this point in the process, in some examples, the method includes removing a portion of the substrate such that the trenches extend through the substrate from the first surface of the substrate to the second surface of the substrate, but in other examples, that aspect is followed by bonding a carrier wafer to the second surface of the substrate. In the example illustrated in FIG. 10, a carrier wafer 1015 is bonded to the surface of the substrate 1005 (850), and then, a portion of the substrate 1005 is removed so the trenches 1051 extend through the substrate vertically (840).

Referring to FIG. 10B, a germanium layer 1032 (e.g., silicon germanium, Si(x)Ge(1-x)) is formed (e.g., deposited) on a surface of the sensor 1035 that does not include the diodes 1050 (860). An intermediate one or more layers of isolation oxide (e.g., ˜20-˜30 nm and/or of another dielectric material (e.g., SiO2, NiO, Si3N4, etc.), can be deposited on the sensor 1035 surface before and/or after depositing the germanium layer 1032 (910, 920, 930). As discussed herein, the germanium layer 1032 can be formed utilizing a variety of techniques, including but not limited to, PECVD, sputter, e-beam evaporation, crystalline growth and etching, and radical activation bonding in vacuum. Examples of various parameters under which these techniques can be utilized in manufacturing certain of the biosensors described herein are provided in Table 1.

Returning to FIG. 10B, a dielectric stack 1025 is formed above a surface of the germanium layer 1032 (870). The stack can be formed on the germanium layer 1032 or on a top layer of one or more isolation oxide layers deposited on the germanium layer 1032. The dielectric stack can be formed from multiple oxide layers and/or layers of another dielectric material (e.g., NiO, SiO2, Si3N4, tantalum pentoxide, etc.) and a sensor (e.g., CMOS) compatible metal (e.g., aluminum, tantalum, etc.). By way of example and not to impose or suggest any limitations, the compatible metal can be approximately 200 nm to approximately 500 nm.

FIG. 11 is a workflow 1100 that illustrates an examples of a method for manufacturing or fabricating a biosensor that includes a germanium layer for LICR and filtering; this workflow uses a commercially available or off-the-shelf sensor (e.g., CMOS). As illustrated in FIGS. 11-12, method of fabricating and/or manufacturing the biosensors illustrated herein that include off-the-shelf sensors include covering off the shelf image sensors with a thin layer of germanium.

As illustrated in FIG. 11, the example of this method includes obtaining an off-the-shelf image sensor (1100). In some examples, this sensor is backside-illuminated and has a deep trench. This workflow 1100 includes forming a germanium (e.g., silicon germanium, Si(x)Ge(1-x)) layer above the image sensor. The process described in FIG. 9 can be utilized to form this layer. Thus, the resultant biosensor can include intermediary isolation oxide or of another dielectric material (e.g., SiO2, NiO, Si3N4, etc.) between a sensor surface and the germanium (e.g., silicon germanium, Si(x)Ge(1-x)) layer and between the germanium (e.g., silicon germanium, Si(x)Ge(1-x)) layer and the dielectric stack. In some examples, these isolation oxide layers are approximately 20 nm to approximately 30 nm. As with certain of the other workflows described herein the workflow 1100 of FIG. 11 also includes forming a dielectric stack above the germanium (e.g., silicon germanium, Si(x)Ge(1-x)) layer (1130). In some examples, the stack is formed directly on the germanium layer and in other examples, it is formed on a top layer of one or more oxide layers and/or one or more dielectric layers separating the dielectric stack from the germanium layer. The dielectric stack can be formed from multiple oxide layers and/or dielectric layers of another material (e.g., NiO, SiO2, tantalum pentoxide, Si3N4, etc.) and a sensor (e.g., CMOS) compatible metal (e.g., aluminum, tantalum, etc.). By way of example and not to impose or suggest any limitations, the compatible metal can be approximately 200 to approximately 500 nm. The manner in which a germanium layer is deposited on the sensor can be accomplished utilizing one or more of the techniques summarized in Table 1.

FIG. 12 depicts various aspects of the workflow 1100 of FIG. 11 and the workflow 900 of FIG. 9, as it relates to the workflow 1100 of FIG. 11 but adds visuals for illustrative purposes in the same way that FIG. 10 illustrates various aspects of the workflows 800, 900 of FIGS. 8-9. Thus, various references to FIGS. 9 and 11 are included in FIG. 12. For ease of understanding, similar numbering is used in FIG. 12 as was utilized in FIG. 3, where possible. The workflow 1200 depicted in FIG. 12 results in a biosensor 1203.

In contrast to FIG. 10, FIG. 12 starts with an off-the-shelf sensor 1255 (e.g., a backside illuminated image sensor with one or more deep trenches) (1110). A layer of germanium 1232 is formed on the sensor 1255 (1120). For example, this germanium layer 1232 can be deposited utilizing one or more of the techniques summarized in Table 1. In some examples, before the germanium layer 1232 is formed, one or more layers of isolation oxide or of another dielectric material (e.g., SiO2, NiO, Si3N4, etc.) 1224 are formed over the sensor 1255 such that these one or more layers of isolation oxide 1234 separate the germanium layer 1232 (which provides LICR and acts as an emission filter) from the sensor 1255 (910).

A dielectric stack 1225 is formed on a top surface of the sensor package (1130). For example, the dielectric can be formed, in some examples, on the germanium layer 1232. In other examples, an additional one or more layers of isolation oxide and/or of another dielectric material (e.g., SiO2, NiO, Si3N4, etc.) 1224 are formed over the germanium layer 1232, forming a barrier between the germanium layer 1232 and a dielectric stack 1225. Thus, in these examples, the dielectric stack is formed on a top layer of these additional one or more layers of isolation oxide and/or of another dielectric material (e.g., SiO2, NiO, Si3N4, etc.) 1224. In some examples, the dielectric stack 1225 is comprised of multiple oxide layers and/or of another dielectric material (e.g., NiO, SiO2, Si3N4, tantalum pentoxide, etc.) 1228 and sensor compatible metal (e.g., aluminum, tantalum, etc.) 1236. The dielectric stack 1225 includes wells 1212 and reaction sites 1214. The sensor 1255 can comprise one or more sensors which are vertically centered under a corresponding well 1212 and reaction site 1214, such that each sensor forms sensing pair with a corresponding reaction site 1214. In the resultant biosensor 1203 of FIG. 12, the pitch distance (P) represents the distance between a central axis of a well 1212 and each adjacent well 1212.

It is desirable to reduce crosstalk in sensors, including in biosensors utilized in flow cells, as crosstalk adversely affects performance. A traditional way in which crosstalk can be reduced in an apparatus with an image sensor, such as a flow cell, is by physically constraining transmission of light by embedding various light guides in the sensor, including but not limited to curtain structures, light pipes, and/or optical waveguides and/or micro-lenses. These structures direct light emitted from a corresponding reaction site directly downwardly toward an image sensor that forms a sensing pair with the reaction site. These structures reduce crosstalk by physically blocking light to provide tailored absorption of light that might otherwise result in crosstalk. As is the case with the LICR layer, discussed in later examples, the physical structures reduce crosstalk.

Because of the manufacturing complexities and structural limitations associated with sensor devices that include structural elements (e.g., curtain structures, light pipes, and/or optical waveguides) to reduce crosstalk, it is desirable to provide a version of a biosensor that suitably prevents or reduces the occurrence of optical crosstalk, without presenting the manufacturing complexity and expense associated with these structures and without constraining certain parameters of the sensor, including pitch distance, which will be discussed herein. Rather than integrate what are sometimes complex structures into sensing devices to reduce crosstalk, such as the aforementioned curtain structures, light pipes, and/or optical waveguides, which increase the cost and complexity of the sensor devices, examples of sensor devices, also referred to herein as biosensors, described herein instead include at least one layer of germanium that provides LICR and/or an emission filter. LICR features, such as the germanium layer, integrated into biosensors described herein do not completely eliminate crosstalk, but, rather, provide tailored absorption of light that might otherwise result in crosstalk. LICR examples suitably prevent or reduce the occurrence of optical crosstalk, without presenting the manufacturing complexity and expense associated with the structural elements (e.g., curtain structures, light pipes, and/or optical waveguides) and without constraining a reduction of pitch distance in a biosensor. Utilizing layers to reduce crosstalk, rather than structures, provide blankets that are uniform in the x-y plane.

Described herein are structures of examples of sensor devices (e.g., biosensors) which include this at least one germanium layer for LICR and as an emission filter, methods of using these sensor devices, and methods of manufacturing these sensor devices. Manufacturing examples herein include both biosensors that include off-the-shelf sensor components, such as an off-the shelf-CMOS, as well as methods that involve forming a custom sensor or CMOS. As noted above, utilizing a sensor device with a germanium layer for LICR instead of a complex structure (e.g., curtain structures, light pipes, and/or optical waveguides) reduces development cost and turn-around time of sensor-based sequencers, including, but not limited to, CMOS-based sequencers. The methods of manufacture utilized to manufacture sensors with curtain structures, light pipes, and/or optical waveguides, involve a high level of customization, increase cost, and, from a performance perspective, add difficulty to increasing reaction site density, when compared with methods of manufacture of biosensors with a germanium layer. The off-the-shelf-sensors utilized in the examples herein include both FSI and BSI sensors.

In various traditional crosstalk mitigation methods, the structures include one or more of an organic filter and/or a metallic curtain that surrounds the organic filter to reduce crosstalk. In lieu of this organic filter and/or other structural elements spanning the height of the biosensors for crosstalk reduction (e.g., curtain structures, light pipes, and/or optical waveguides) to reduce crosstalk, the examples herein employ germanium (in a non-limiting example, ˜300-˜330 nm) as a filter and LICR medium. Some benefits and advantages of the methods and apparatuses described herein over biosensors with the aforementioned organic filter and/or complex structures are that: 1) the devices produced using the methods described herein can employ off-the-shelf image sensors (including both FSI and BSI sensors); 2) the methods herein are less complex and thus produce devices with fewer complexities; 3) the devices produced with the methods described herein can enable further density increases while shrinking pixel pitch; and 4) based on the methods producing less complex devices and producing them with fewer complex steps, supply chain risks are reduced. Other benefits and advantages may be discussed herein or may be apparent from this disclosure. One of the reasons that germanium is effective in crosstalk reduction in biosensors is that it has a high absolute difference in absorption between red and green. Other materials with similar high absolute difference in absorption between different ranges of wavelengths, for example, between red and green, may be suitable.

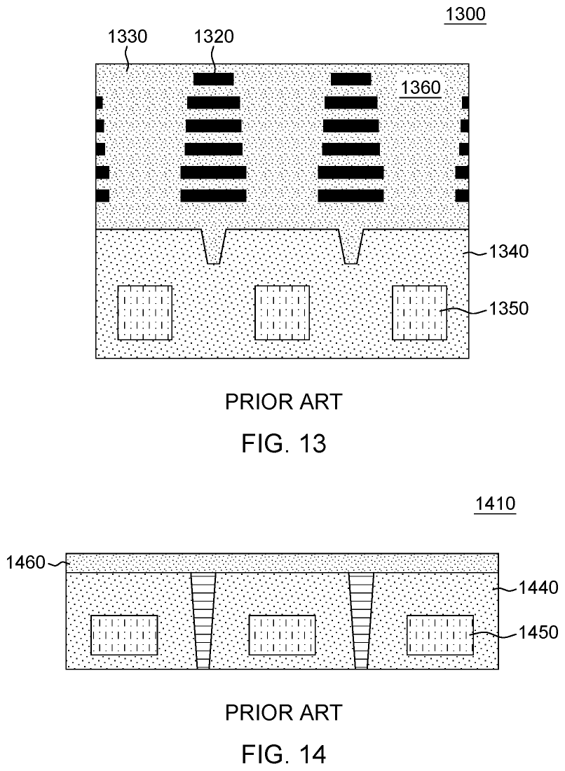

Examples herein include both BSI and FSI sensors or chips that are utilized as a base for fabricating a biosensor. FIG. 13 is an example of an FSI sensor or chip that can be utilized in certain of the examples herein. FIG. 14 is an example of a BSI sensor or chip that can be utilized in certain of the examples herein. The examples provided of biosensors herein are non-limiting and merely provided as possible designs for biosensors that utilize a silicon germanium layer to reduce crosstalk. Certain of the FSI-based examples include a silicon germanium layer with a top surface on a contiguous horizontal plane (e.g., the surface of the layer is even or approximately even) while other of the FSI-based examples include a silicon germanium layer into which trenches are formed. Similarly, the BSI-based designs also include designs with a silicon germanium layer with an even or approximately even upper surface and designs with a silicon germanium layer into which trenches are formed. Described herein are the structures of this designs as well as various methods of forming these biosensor examples.

Examples herein have various similarities in both structure and method of manufacture, regardless of whether the base sensor is a BSI sensor or an FSI sensor. For this reason, examples of off-the shelf sensors that can be integrated into these examples are illustrated in FIGS. 13 and 14.

FIG. 13 is an example of an FSI sensor or chip 1310 (e.g., a CMOS) that can be integrated into various examples herein. The FSI sensor includes one or more PN (positive-negative) junction sensors, referred to herein as diodes 1350 (considered elementary building blocks of semiconductors). The diodes 1350 are situated in a substrate 1340, in this example, the substrate 1340 can be comprised of silicon. PN junction sensors 1350 are also referred to as diodes. The FSI sensor 1310 includes various internal electrical connections formed using various conductive elements 1320, in this non-limiting example, comprised of metal. Additionally, to reduce crosstalk, the FSI sensor 1310 includes light pipes 1330 between the conductive elements 1320. The light pipes 1330 and the conductive elements 1320 are all oriented in a low-index layer 1360, which in some examples is comprised of oxide.

FIG. 14 is an example of a BSI sensor 1410 (e.g., a CMOS) that can be integrated into various examples herein. The BSI sensor 1410 includes diodes 1450 (e.g., PN junction sensors) oriented in a substrate 1440 that can include silicon. The BSI sensor 1410 also includes a low-index layer 1460, which in some examples is comprised of oxide.

Certain examples herein are depicted with nanowells, however, nanowells are only one example of structures that can be utilized atop a biosensor to accomplish various aspects of the functionalities of the biosensors. Thus, when the examples herein depict nanowells, one of skill in the art will understand that different structures can be substituted as the nanowells as they may not be required or alternative structures may prove suitable in certain implementations.

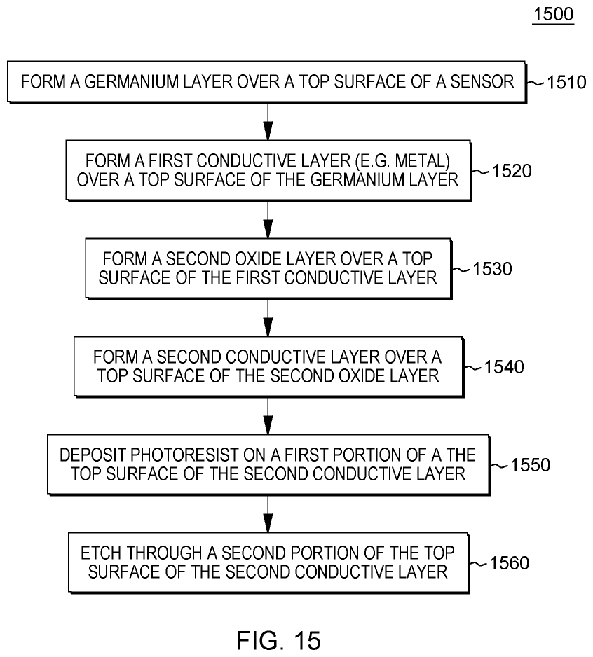

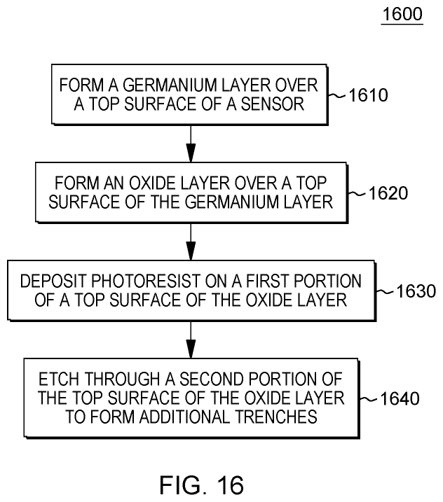

Whether the sensor utilized in the resultant biosensor is a BSI sensor or an FSI sensor, certain of the techniques for fabricating the examples herein are similar if not identical. Before discussing various examples and the specifics of fabricating these examples, FIGS. 15-16 are workflows 300, 400 that review, generally, various aspects of fabricating the biosensors described herein.

As illustrated in FIG. 15, in some examples herein, the method of forming a biosensor includes forming a germanium layer over a top surface of a sensor (1510). As illustrated in FIGS. 13-14, the sensor includes one or more diodes 1350, 1450 (e.g., PN junction sensors) in a substrate 1340, 1440 and an oxide layer (e.g., a layer made of a material with a low refractive index 1360, 1460) formed over a top surface of the substrate 1340, 1440. The oxide layer can include electrically conductive materials (e.g., FIG. 13, conductive elements 1320). For the sake of clarity, the low-index layer that is part of the sensor can be referred to as a first oxide layer or a first low index layer and any subsequent oxide layers or other layers of a material with a low refractive index added in this method can be numbered sequentially. The biosensors in some examples can include both FSI and BSI sensors. Both BSI and FSI sensors can be CMOSs.

The germanium layer can include silicon. Various techniques can be utilized to form this germanium layer in different examples. For example, the layer can be formed by sputtering silicon-germanium onto the top surface of the first oxide layer (the oxide layer included in the sensor).

In other examples, the germanium layer on the sensor is formed by a combination of aspects. First, one can deposit photoresist on a first portion of the top surface of the first oxide layer. Then, one can etch through a second portion of the top surface of the first oxide layer (the photoresist is not deposited on the second portion of the top surface of the first oxide layer) to form one or more trenches in the first oxide layer.

Once the etching is complete, one can deposit a crosstalk mitigating substance above the first oxide layer (e.g., oxide, nitride, and silicon), which includes filling the one or more trenches in the first oxide layer. One can planarize the crosstalk mitigating substance such that a portion of the crosstalk mitigating substance forms a contiguous surface with the first portion of the top surface of the first oxide layer. One can then remove the photoresist. Once the photoresist (which preserved the surface as described) is removed, one can deposit a layer of silicon germanium on the top surface of the first oxide layer. CMP can be utilized to perform the planarization. Various methods and techniques can be utilized to remove the photoresist. For example, one can remove the photoresist utilizing a combination of a plasma resist strip followed by a SPM (sulfuric peroxide mix) or other chemical wet cleaning process to remove the remaining residue. In some examples, an etching process, including but not limited to, plasma etching, can be utilized to remove the photoresist. In some examples, after a chemical process is utilized, the remainder of the residue can be removed via etching.

Depending on the techniques used to form the germanium layer, the nature and shape of the layer can vary. In some examples, wherein the structure includes a conductive layer (e.g., metal, which will be described in greater detail herein) forming this germanium layer includes sputtering a conductive layer on the top surface of the first oxide layer. This example can also include depositing photoresist on a first portion of the conductive layer, wherein the second portion of the first oxide layer remains exposed (the photoresist does not cover this portion of the first oxide layer). Based on depositing the photoresist, one can remove a second portion of the conductive layer with etching (the photoresist is not deposited on the second portion of the conductive layer). This etching removes the top surface of the first oxide layer and the first portion of the conductive layer. After these structural changes are implemented, one can deposit a layer of silicon germanium on the top surface of the first oxide layer.

Another example that results in a germanium layer of a distinct configuration is a method where forming the germanium layer over a top surface of the sensor includes depositing photoresist on a first portion of the top surface of the first oxide layer. Based on depositing the photoresist, this example of the method includes etching through a second portion of the top surface of the first oxide layer (the photoresist is not deposited on the second portion of the top surface of the first oxide layer) to form one or more trenches in the first oxide layer. The method then includes depositing the germanium layer above the first oxide layer. This depositing action partially fills the one or more trenches in the first oxide layer rendering the one or more trenches shallower than prior to the depositing. The method then includes depositing a crosstalk mitigating substance (e.g., oxide, nitride, and silicon), above the germanium layer, wherein the depositing fills a remainder of the one or more trenches in the first oxide layer (the germanium did not fill the entirety of the trenches). The method then includes planarizing (e.g., utilizing CMP) the crosstalk mitigating substance such that the top surface of the germanium layer is a contiguous surface that includes a portion of the crosstalk mitigating substance and the first portion of the top surface of the first oxide layer.

Returning to FIG. 15, the method can include forming a first conductive layer (e.g., metal) over a top surface of the germanium layer (1520). The method can also include forming a second oxide layer over a top surface of the first conductive layer (1530). The method can further include forming a second conductive layer (e.g., metal) over a top surface of the second oxide layer (1540). Additionally, the method can include depositing photoresist on a first portion of a top surface of the second conductive layer (1550). In some examples, one can form a passivation layer on the first portion of the top surface of the second conductive layer. A silicon layer can be formed on the top surface of the second oxide layer.

Continuing with the workflow 1500 of FIG. 15, the method can also include etching through a second portion of the top surface of the second conductive layer (1560). In this example, the photoresist is not deposited on the second portion of the top surface of the second conductive layer, a portion of the second oxide layer, and a portion of the first conductive layer. Thus, the etching forms one or more trenches. These trenches can be positioned above at least one diode of the one or more diodes, on a vertical axis extending from a bottom surface of the sensor to the top surface of the second oxide layer. In some examples, nanowells can be formed in the trenches.

FIG. 16, like FIG. 15, is a generalized example of a workflow 1600 for forming a biosensor that includes some aspects that can be incorporated into various examples of methods disclosed herein. As with FIG. 15, the sensor that is utilized in various aspects of this workflow 1600 can be either a BSI or an FSI sensor (e.g., FIG. 13, 1310, FIG. 14, 1410) each of which can be a CMOS. The sensor utilized in this workflow 1600 to form a biosensor includes a substrate (e.g., FIG. 13, 1340, FIG. 14, 1440) comprising one or more diodes (e.g., FIG. 13, 1350, FIG. 14, 1450) and an oxide layer (e.g., FIG. 13, 1360, FIG. 14, 1460) formed over a top surface of the substrate. The oxide layer, which can be understood as a first oxide layer, just for clarity, can comprise electrically conductive materials or components (e.g., FIG. 13). As illustrated in the workflow 1600, the method can include forming a germanium layer over a top surface of a sensor (1610). Forming the germanium layer can be accomplished by depositing photoresist on a first portion of a top surface of the oxide layer. In some examples, one can etch through a second portion of the top surface of the oxide layer (the photoresist is not deposited on the second portion of the top surface of the oxide) to form one or more trenches. As will be illustrated in greater detail herein, these trenches can each be positioned above a space between at least one diode and another diode of the one or more diodes, on a vertical axis extending from a bottom surface of the sensor to the top surface of the germanium layer.

The aspect in the methods described herein of forming the germanium layer on the top surface of the sensor (1610) can include various sub-aspects. In one example, the formation of this layer includes depositing silicon germanium on the top surface of the first oxide layer (this is the oxide layer that is part of the initial sensor). This example includes depositing a conductive layer on the top surface of the silicon germanium. One can also deposit a photoresist on a first portion of the conductive layer. The photoresist preserves the parts of the layer upon which it is deposited so one can etch through a second portion of the conductive layer, the portion upon which the photoresist is not deposited, to remove this second portion of the conductive layer. After this etching is complete, the top surface of the germanium layer includes the first portion of the conductive layer and a portion of the silicon germanium.

Another variation in forming the germanium layer involves depositing a conductive layer on the top surface of the sensor and depositing photoresist on a first portion of the conductive layer. After depositing the photoresist, one can etch through a second portion of the conductive layer (i.e., the portion of the layer upon which the photoresist is not deposited) and the etching removes the second portion of the conductive layer. Once the etching is complete, this example of the method proceeds to deposit silicon germanium over a portion of the first oxide layer and the first portion of the conductive layer.

Returning to FIG. 16, the example workflow 1600 can include forming an oxide layer (a second oxide layer) over a top surface of the germanium layer (1620). The method also can include depositing photoresist on a first portion of a top surface of the second oxide layer (1630). Depositing the photoresist enables etching through a second portion of the top surface of the second oxide layer (the photoresist is not deposited on the second portion of the top surface of the second oxide layer) to form additional trenches (1640). These additional trenches can each be positioned above at least one diode of the one or more diodes, on a vertical axis extending from a bottom surface of the sensor to the top surface of the second oxide layer.

As with the workflow 1500 of FIG. 15, the workflow 1600 of FIG. 16 can include additional aspects when forming the germanium layer on the top surface of the sensor. For example, one can deposit silicon germanium on the top surface of the first oxide layer (the oxide layer included in the sensor). One can deposit a conductive layer on the silicon germanium. One can also remove portions of these deposits by first, depositing photoresist on a first portion of the conductive layer, and then, etching through a second portion of the conductive layer (the photoresist is not deposited on the second portion of the conductive layer) such that this etching removes the second portion of the conductive layer so that the top surface germanium layer is a surface that includes a portion of the silicon germanium and the first portion of the conductive layer.

Additional aspects can be included in the workflow 1600. For example, one can form a silicon layer on the top surface of the second oxide layer. Additionally, some examples include depositing a conductive layer on the top surface of the second oxide layer. Upon depositing this layer one can deposit photoresist on a first portion of the conductive layer. After depositing the photoresist, one can etch through a second portion of the conductive layer (the photoresist is not deposited on the second portion of the conductive layer) to remove the second portion of the conductive layer.

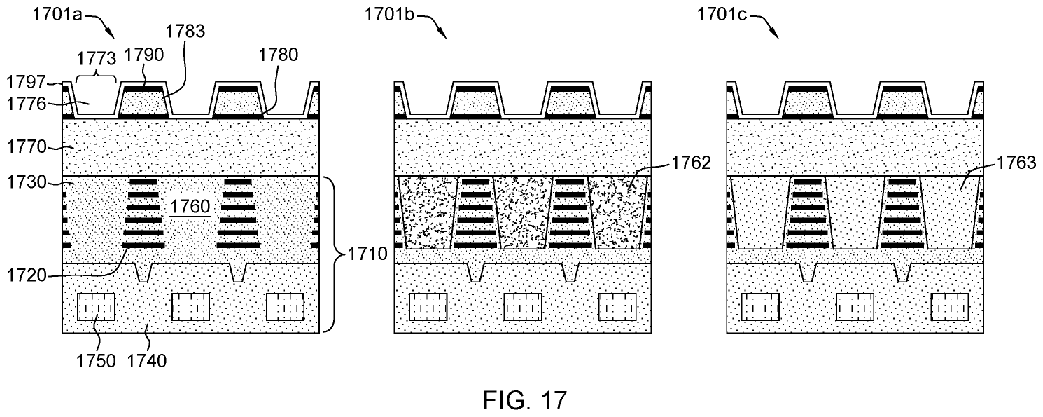

FIGS. 15-16 illustrate how various aspects can be assembled differently to form various biosensors. Certain aspects are included in different examples herein but are combined and/or configured differently. FIGS. 17-27 illustrate various biosensors and/or formation of these biosensors, where the initial sensor used as a building block is an FSI sensor. FIGS. 15-30 illustrate various biosensors and/or formation of these biosensors, where the initial sensor used as a building block is a BSI sensor. Common elements in these biosensors, whether the initial sensor is a BSI sensor or an FSI sensor, can include the sensor (e.g., FIG. 13, 1310, FIG. 14, 1410), various low index oxide layers, various conductive (e.g., metal) layers, various germanium layers, and in some examples, high index oxide and/or nitride layers and/or silicon layers. The configuration of the germanium layer can vary from a blanket layer to an etched layer (not consistent over the length of the biosensor), to a layer that is situated to fill a trench in another layer.

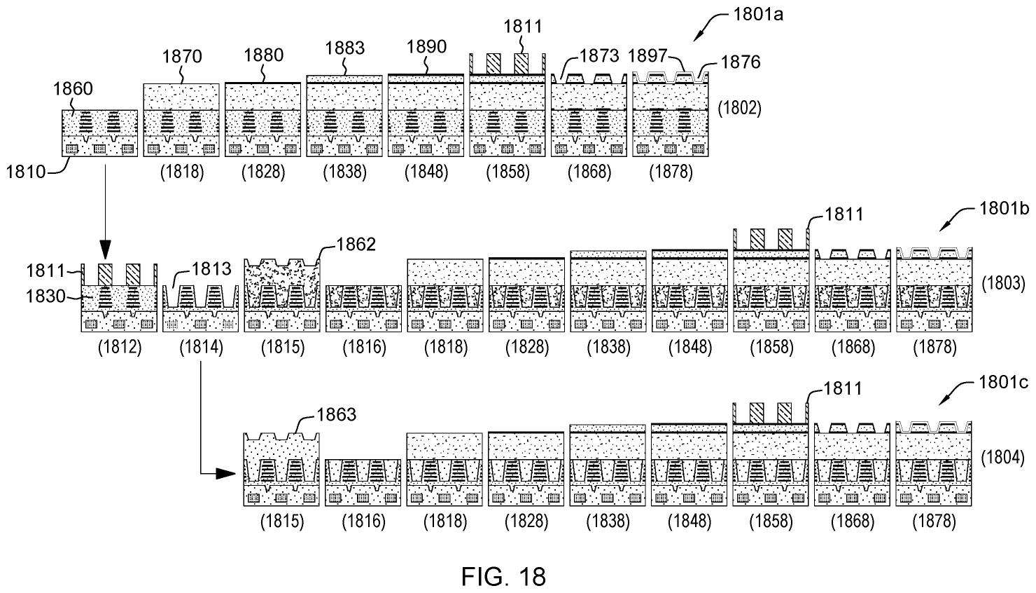

FIGS. 17, 19, and 21 illustrate eight different examples of biosensors that include FSI sensors. In each of these examples, the biosensors include a consistent layer, which can be referred to as a blanket layer, that includes germanium (e.g., the top and bottom surfaces of the germanium layer share horizontal parallel planes). FIGS. 18, 20, and 22 illustrate examples of methods utilized to make the sensors in FIGS. 17, 19, and 21, respectively.

FIG. 17 illustrates three configurations for a biosensor 1701a-1701c where the variation in each is what material, if any, fills a lightpipe 1730 structure in a low (refractive) index layer 1760 (e.g., oxide). In each case, the biosensor 1701a-1703c includes a sensor 1710, in these examples, an FSI sensor (which includes the lightpipe structure, as opposed to a BSI sensor, which does not). The sensor 1710 includes a substrate 1740 comprising one or more diodes 1750 and the aforementioned low (refractive) index layer 1760 (e.g., oxide). The low index layer 1760 includes electrically conductive materials 1720 (e.g., metal). Each of the biosensors 1701a-1701c includes a germanium layer 1770 formed over a top surface of the sensor 1710, a first conductive layer 1780 (e.g., metal) formed over a top surface of the germanium layer 1770, a second low (refractive) index layer (e.g., oxide) 1783 formed over a top surface of the first conductive layer 1780, and a second conductive layer 1790 (e.g., metal) formed over a top surface of the second low index layer 1783. In these examples, the second conductive layer 1790, the second low index layer 1783, and the first conductive layer 1780, include trenches 1773. These trenches 573 can form nanowells 1776. Each trench (e.g., nanowell) is positioned above at least one diode of the diodes 1750 (i.e., on a vertical axis extending from a bottom surface of the sensor 1710 to the top surface of the second low index layer 1783). In one configuration 1701a, the lightpipe 1730 is filled with the original low (refractive) index layer 1760, hence, no alternation is made to the off-the-shelf FSI sensor. In a second configuration 1701b, the lightpipe 1730 is filled with the a high (refractive) index layer 1762 (e.g., oxide and/or nitride). In a third configuration 1701c, the lightpipe 1730 is filled with silicon 1763. Each biosensor 1701a-1701c is topped with a passivation layer 1797, which in these examples, can be comprised of silicon.

FIG. 18 illustrates workflows 1802-1804 that can be used to form the sensors 1701a-1701c of FIG. 17, labelled 1801a-1801c in FIG. 18. Because the difference between these sensors 1801a-1803a are the materials that fill the lightpipes 1830, there are variations in the workflows, specifically, the latter two workflows 1803-1804 include an aspect of removing the original low index layer 1860 by depositing a photoresist and etching the areas in the layer that the photoresist does not cover, and then, filling the etched trenches with the alternative material, a high (refractive) index layer 1862 (e.g., oxide and/or nitride) or silicon 1863.

Each workflow in FIG. 18 commences with a sensor (e.g., a FSI CMOS) 1810. In the first workflow 1802, one forms a germanium layer 1870 over a top surface of the sensor 1810 (1818). In the second and third workflow 1803-1804, before depositing the germanium (1818), one replaces the low index material 1860 in the lightpipe 1830 portion of the sensor 1810 and then, deposits a germanium layer 1870 (1818). In the second and third workflow 1803-1804, the low index material in the lightpipe portion of the sensor is replaced first, by depositing a photoresist (e.g., utilizing photolithography) 1811 on a first portion of a top surface of the low index material (the portions that are not above the lightpipe) (1812), and then, by etching where the photoresist is not deposited to form trenches 1813 in the lightpipe 1830 areas, and removing the photoresist (e.g. utilizing resist strips, chemical cleaning, and/or etching) (1814). One then fills the trenches 1813 with the material of choice (1815) (e.g., utilizing PECVD); in the second workflow 1803, this material is a material with a high refractive index 1862 (e.g., an oxide of nitride including but not limited to SiO), and in the third workflow 1804, this material is silicon 1863. After the trenches are filled with a material, the top of the material (e.g., a material with a high refractive index 1862 or silicon 1863) can be planarized (e.g., using CMP) (1816) and then, one can deposit the germanium layer 1870 (1818). This germanium layer 1870 (e.g., SiGe) can be formed using a sputter technique. As discussed earlier, depending upon the desired topography of the resultant biosensor (e.g., its intended use), planarization of its surfaces, including the use of CMP, can be omitted.

In each of the workflow 1803-1804 of FIG. 18, once the germanium layer 1870 is deposited (1818), one can form a first conductive layer 1880 (e.g., metal) over a top surface of the germanium layer 1870 (e.g., using a technique including but not limited to, metal sputtering) (1828). This example of the method can then include forming a second low index layer 1883 (e.g., an oxide layer) over a top surface of this first conductive layer 1880 (1838). Atop the second low index layer 1883, one can form a second conductive layer 1890 (1848).

As discussed above, certain of the biosensors formed with the methods discussed herein include nanowells, which perform some of the desired functionality. Thus, the workflows 1802-1804 of FIG. 18 include aspects that can be utilized to form nanowells. However, chemistry can also be applied to the surface of the biosensor after forming the second conductive layer over a top surface of the second low index layer (1848), instead of proceeding to form trenches on the top surface of the biosensor for use as nanowells.

Returning to FIG. 18, to form nanowells 1876, one can deposit photoresist 1811 on a first portion of a top surface of the second conductive layer 1890 (1858) (e.g., using photolithography). Portions of the surfaces upon which photoresist 1811 is deposited are preserved during a subsequent etching process. One can then etch the portions of the second conductive 1890 layer that are not covered by the photoresist 1811 (e.g., utilizing an oxide and metal etching process) to form trenches 1873 in both conductive layers 1880, 1890 and the second low index layer 1883, exposing parts of the germanium layer 1870 and then remove the photoresist (e.g., utilizing resist strips, chemical cleaning, and/or etching) (1868). In some examples, various chemistries can then be applied to the trenches. A passivation layer 1897 can be deposited atop the top surface of the structure, which can be a silicon layer (1878).

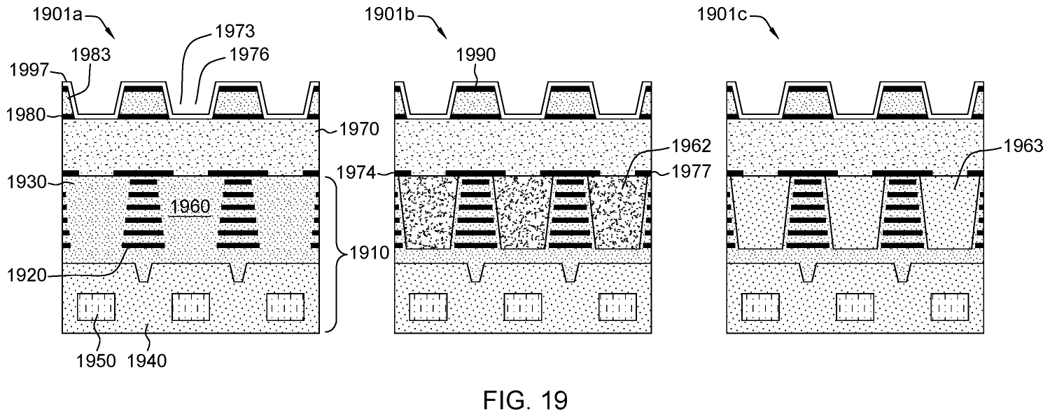

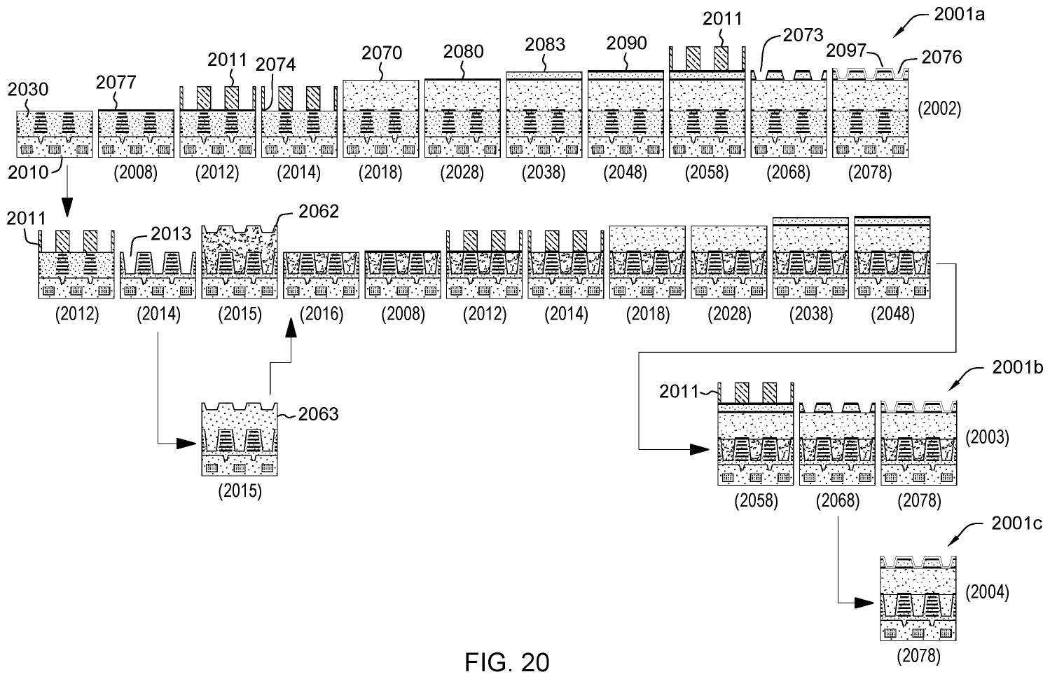

FIG. 19, like FIG. 17, includes three examples 1901a-1901c of biosensors that can be formed using the techniques described herein. The biosensors 1901a-1901c in FIG. 19 additionally includes blocking (conductive) elements 1974 (e.g., metal) from a blocking (conductive) layer 1977 that is formed atop the sensor 1910 and then, partially removed (as illustrated and discussed in more detail in FIG. 20). Like in FIG. 17, the biosensor 1901a-1901c in FIG. 19 vary from each other in what material, if any, fills a lightpipe 1930 structure in a low (refractive) index layer 1960 (e.g., oxide). The sensor 1910 in each example is an FSI sensor. The sensor 1910 includes a substrate 1940 comprising one or more diodes 1950 and a low (refractive) index layer 1960 (e.g., oxide). The low index layer 1960 includes electrically conductive materials 1920 (e.g., metal). Each of the biosensors 1901a-1901c includes a germanium layer 1970 formed over a surface that includes some of the low (refractive) index layer 1960 and conductive elements 1974 (left over from a (conductive) blocking layer 1977), a first conductive layer 1980 (e.g., metal) formed over a top surface of the germanium layer 1970, a second low (refractive) index layer (e.g., oxide) 1983 formed over a top surface of the first conductive layer 1980, and a second conductive layer 1990 (e.g., metal) formed over a top surface of the second low index layer 1983. In these examples, the second conductive layer 1990, the second low index layer 1983, and the first conductive layer 1980, include trenches 1973. These trenches can 1973 can form nanowells 1976. Each trench (e.g., nanowell) is positioned above at least one diode of the diodes 1950 (i.e., on a vertical axis extending from a bottom surface of the sensor 1910 to the top surface of the second low index layer 1983). In one configuration 1901a, the lightpipe 1930 is filled with the original low (refractive) index layer 1960. In a second configuration 1901b, the lightpipe 1930 is filled with the a high (refractive) index layer 1962 (e.g., oxide and/or nitride). In a third configuration 1901c, the lightpipe 1930 is filled with silicon 1963. Each biosensor 1901a-1901c, in these examples, is topped with a passivation layer 1997, which in these examples, can be comprised of silicon.

FIG. 20 includes various aspects of workflows 2002-2004 used to form the biosensors 1901a-1901c in FIG. 19. Unlike in FIG. 18, these workflows 2002-2004 include forming a blocking layer 2077, which is an additional conductive layer (e.g., metal) before forming the germanium layer 2070. In cases where elements of the sensor 2010 are altered before forming a germanium layer 2070 (e.g., replacing materials in the lightpipe 2030), these aspects are performed in advance of forming and removing portions of the blocking layer 2077.

Each workflow 2002, 2003, 2004 in FIG. 20 commences with a sensor (e.g., a FSI CMOS) 2010. In the first workflow 2002, one forms a blocking layer 2077 (e.g., using a metal sputtering technique) over a top surface of the sensor 2010 (2008). One then can deposit photoresist 2011 (e.g., utilizing photolithography) atop portions of the blocking layer 2077 (2012). Utilizing a technique including but not limited to etching, including mechanical etching, remove portions of the blocking layer 2077 (those not covered with the photoresist 2011), leaving blocking elements 2074 (which assist in crosstalk mitigation) and then remove the photoresist (e.g., utilizing resist strips, chemical cleaning, and/or etching) (2014). One forms a germanium layer 2070 over a top surface of the sensor 2010, which now includes the blocking elements 2074 (2018). In the second and third workflow 2003-2004, before forming the blocking layer 2077 (2008), the workflow includes depositing photoresist 2011 (2012), etching the blocking layer 2077 to leave blocking elements 2074 at the top surface of the sensor 2010, removing the photoresist, and depositing the germanium (2018), to replace the low index material in the lightpipe 2030 portion of the sensor 2010 and then, depositing a germanium layer 2070 (2018).

In the second and third workflow 2003-2004, the low index material 2030 in the lightpipe portion of the sensor is replaced first, by depositing a photoresist (e.g., utilizing photolithography) 2011 on a first portion of a top surface of the low index material (the portions that are not above the lightpipe) (2012), and then, by etching where the photoresist 2011 is not deposited to form trenches 2013 in the lightpipe 2030 areas, and then removing the photoresist (e.g., utilizing resist strips, chemical cleaning, and/or etching) (2014). One then fills the trenches 2013 with the material of choice (2015) (e.g., utilizing PECVD), in the second workflow 2003; this material is a material with a high refractive index 2062 (e.g., an oxide of nitride including but not limited to SiO), and in the third workflow 2004, this material is silicon 2063. After the trenches are filled with a material, the top of the material (e.g., a material with a high refractive index 2062 or silicon 2063) can be planarized (e.g., using CMP) (2016) and then, one can form the blocking layer 2077 (2008), deposit photoresist 2011 (2012), etch the blocking layer 2077 to leave blocking elements 2074 at the top surface of the sensor 2010, remove the photoresist, and deposit the germanium layer 2070 (2018). This germanium layer 2070 (e.g., SiGe) can be formed using a sputter technique. As discussed earlier, depending upon the desired topography of the resultant biosensor (e.g., its intended use), planarization of its surfaces, including the use of CMP, can be omitted.

In each of the workflow 2003-2004 of FIG. 20, once the germanium layer 2070 is deposited (2018), one can form a first conductive layer 2080 (e.g., metal) over a top surface of the germanium layer 2070 (e.g., using a technique including but not limited to, metal sputtering) (2028). This example of the method can then include forming a second low index layer 2083 (e.g., an oxide layer) over a top surface of this first conductive layer 2080 (2038). Atop the second low index layer 2083, one can form a second conductive layer 2090 (2048).

As discussed above, certain of the biosensors formed with the methods discussed herein include nanowells, which perform some of the desired functionality. Thus, the workflows 2002-2004 of FIG. 20 include aspects that can be utilized to form nanowells. However, chemistry can also be applied to the surface of the biosensor after forming the second conductive layer over a top surface of the second low index layer (2048), instead of proceeding to form trenches on the top surface of the biosensor for use as nanowells 2076.

Returning to FIG. 20, to form nanowells 2076, one can deposit photoresist 2011 on a first portion of a top surface of the second conductive layer 2090 (2058) (e.g., using photolithography). Portions of the surfaces upon which photoresist 2011 is deposited are preserved during a subsequent etching process. One can then etch the portions of the second conductive layer 2090 that are not covered by the photoresist 2011 (e.g., utilizing an oxide and metal etching process) to form trenches 2073 in both conductive layers 2080, 2090 and the second low index layer 2083, exposing parts of the germanium layer 2070, and remove the photoresist (e.g., utilizing resist strips, chemical cleaning, and/or etching) (2068). In some examples, various chemistries can then be applied to the trenches. A passivation layer 2097 can be deposited atop the top surface of the structure, which can be a silicon layer (2078).

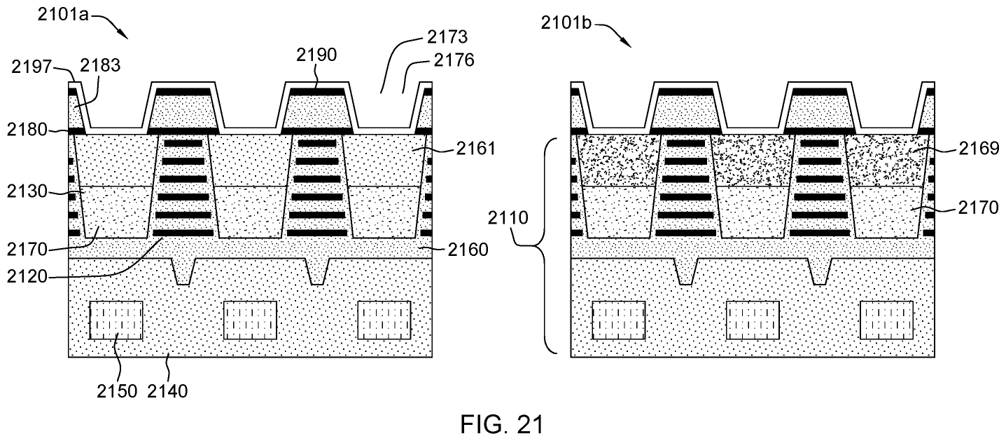

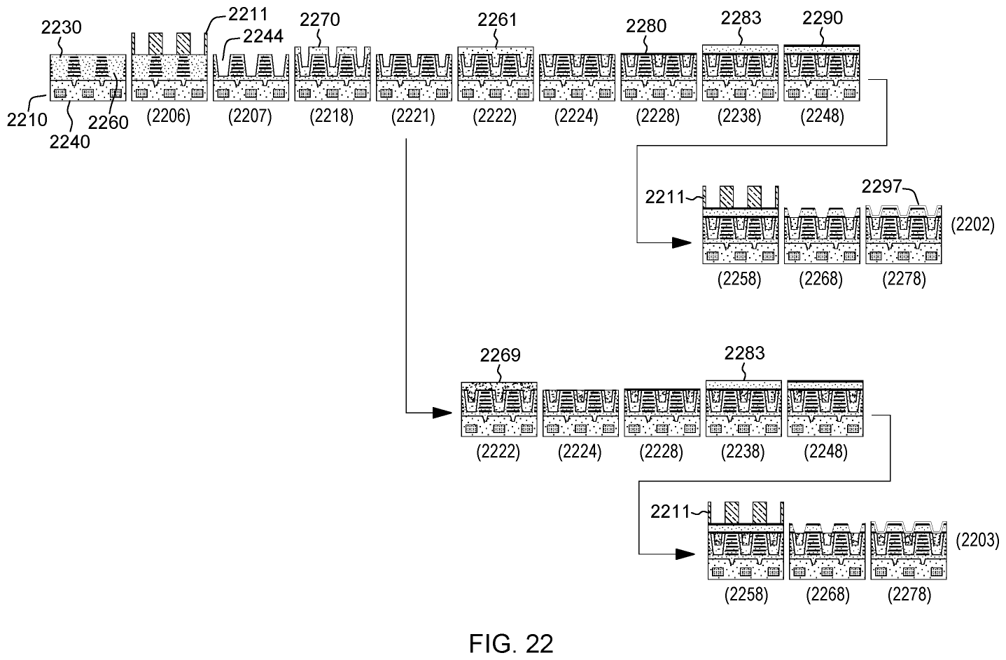

FIG. 21 illustrates two examples 2101a-2101b, where a germanium layer 2170 is deposited in a lightpipe 2130 structure in the sensor 2110. The lightpipe 2130 trench includes silicon 2161 in one example 2101a and a material with a high refractive index 2169 (e.g., oxide and/or nitride) in another example 2101b. Thus, in the first example 2101a, a light pipe can be filled with a combination of silicon and a germanium layer 2170 (e.g., SiGe), and in the second example, the lightpipe is filled with a combination of a material with a high (refractive) index (e.g., oxide and/or nitride) and a germanium layer 2170 (e.g., SiGe). The sensor 2110 in each example 2101a-2101b is an FSI sensor. The sensor 2110 includes a substrate 2140 comprising one or more diodes 2150 and a low (refractive) index layer 2160 (e.g., oxide). The low index layer 2160 (portions of it which are not removed, as illustrated in FIG. 22) to house one of: 1) silicon 2161 or 2) the material with the high refractive index 2169, and the germanium layer 2170, includes electrically conductive materials 2120 (e.g., metal).

Each of the biosensors 2101a-2101b includes a germanium layer 2170 formed in the lightpipe 2130 on part of the remaining low (refractive) index layer 2160, after a portion of the low (refractive) index layer 2160 is removed (specifically, from the lightpipe 2130), a first conductive layer 2180 (e.g., metal) formed over a top surface of the sensor 2110, a second low (refractive) index layer (e.g., oxide) 2183 formed over a top surface of the first conductive layer 2180, and a second conductive layer 2190 (e.g., metal) formed over a top surface of the second low index layer 2183. In these examples, the second conductive layer 2190, the second low index layer 2183, and the first conductive layer 2180, include trenches 2173. These trenches 2173 can form nanowells 2176. Each trench (e.g., nanowell) is positioned above at least one diode of the diodes 2150 (i.e., on a vertical axis extending from a bottom surface of the sensor 2110 to the top surface of the second low index layer 2183). Each biosensor 2101a-2101b is topped with a passivation layer 2197, which in these examples, can be comprised of silicon.

Each workflow in FIG. 22 commences with a sensor (e.g., a FSI CMOS) 2210. In both workflows 2202-2203, the lightpipe 2230 portions of the low index material in the low-index layer 2260 of the sensor 2210 in this lightpipe 2230 area are removed so that the resultant trenches can be filled with a germanium layer 2270 and another material, either silicon 2261 or a material with a high refractive index 2269 (e.g., oxide and/or nitride). To this end, the method, both workflows 2202, 2203 include depositing photoresist 2211 (e.g., utilizing photolithography) atop the sensor 2210 (2206). Where the photoresist 2211 is not deposited, one can utilize an etching technique, including mechanical etching to remove portions of the low-index layer 2260 of the sensor 2210 in the lightpipe 2230 area (2207). The etching, in some examples, leaves a (now thinner) layer of the material comprising low-index layer 2260 on the top surface of the substrate 2240 and trenches 2244 in the lightpipe 2230 area. Upon removing the deposited photoresist, one may fill the trenches by forming a germanium layer 2270 over a top surface of the sensor 2210, which now includes filling approximately one half of the trenches (2218) (e.g., utilizing a SiGe sputter). In some examples, the depth of a trench can be between approximately 3 to approximately 3.5 micrometers. Thus, in this nonlimiting example, one can fill half of the trench with a germanium layer 2270 (e.g., sputtered SiGe) by creating a germanium layer with a thickness of approximately 1.5 to approximately 1.75 micrometers.

After planarizing the surface, (2221) (e.g., using CMP) fill the remainder of the trenches 2244 with either silicon 2261 or a material with a high refractive index 2269 (e.g., oxide and/or nitride) (2222) (e.g., utilizing CVD). The top surface of the resulting structure can then be planarized, e.g., utilizing CMP (2224). The planarized surface includes either silicon 2261 or a material with a high refractive index 2269, germanium 2270 (e.g., SiGe), and portion of the low index material in the low-index layer 2260. As discussed earlier, depending upon the desired topography of the resultant biosensor (e.g., its intended use), planarization of its surfaces, including the use of CMP, can be omitted.

One can form a first conductive layer 2280 (e.g., metal) over a top surface of the sensor 2210 (e.g., using a technique including but not limited to, metal sputtering) (2228). This example of the method can then include forming a second low index layer 2283 (e.g., an oxide layer) over a top surface of this first conductive layer 2280 (2238). Atop the second low index layer 2283, one can form a second conductive layer 2290 (2248).

As discussed above, certain of the biosensors formed with the methods discussed herein include nanowells, which perform some of the desired functionality. Thus, the workflows 2202-2203 of FIG. 22 include aspects that can be utilized to form nanowells. However, chemistry can also be applied to the surface of the biosensor after forming the second conductive layer over a top surface of the second low index layer (2248), instead of proceeding to form trenches on the top surface of the biosensor for use as nanowells 2276.

Continuing with FIG. 22, to form nanowells 2276, one can deposit photoresist 2211 on a first portion of a top surface of the second conductive layer 2290 (2258) (e.g., using photolithography). Portions of the surfaces upon which photoresist 2211 is deposited are preserved during a subsequent etching process. One can then etch the portions of the second conductive 2290 layer that are not covered by the photoresist 2211 (e.g., utilizing an oxide and metal etching process) to form trenches 2273 in both conductive layers 2280, 2290 and the second low index layer 2283, exposing parts of the germanium layer 2270 (2268). One can remove the photoresist (e.g., utilizing resist strips, chemical cleaning, and/or etching). In some examples, various chemistries can then be applied to the trenches. A passivation layer 2297 can be deposited atop the top surface of the structure, which can be a silicon layer (2278).

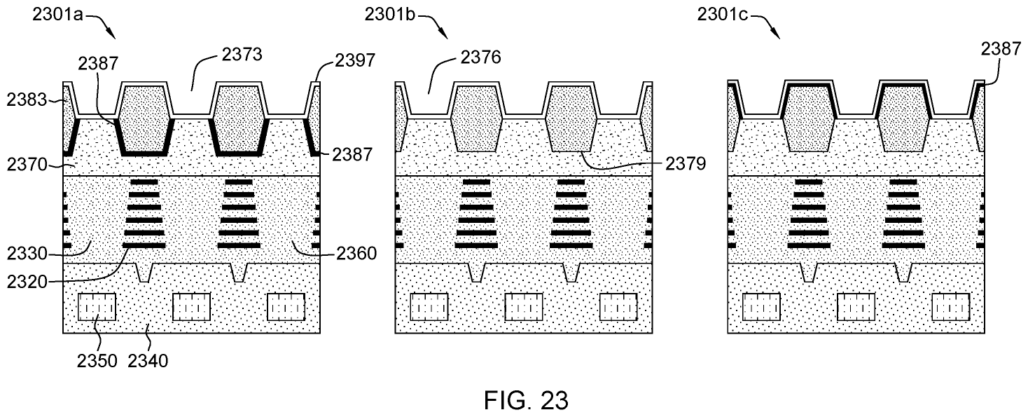

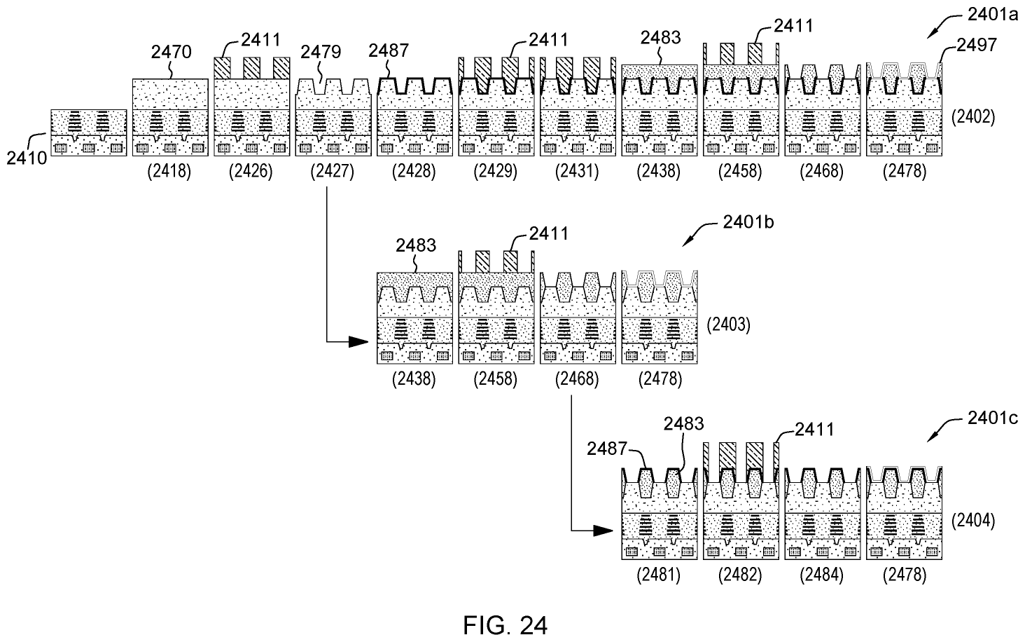

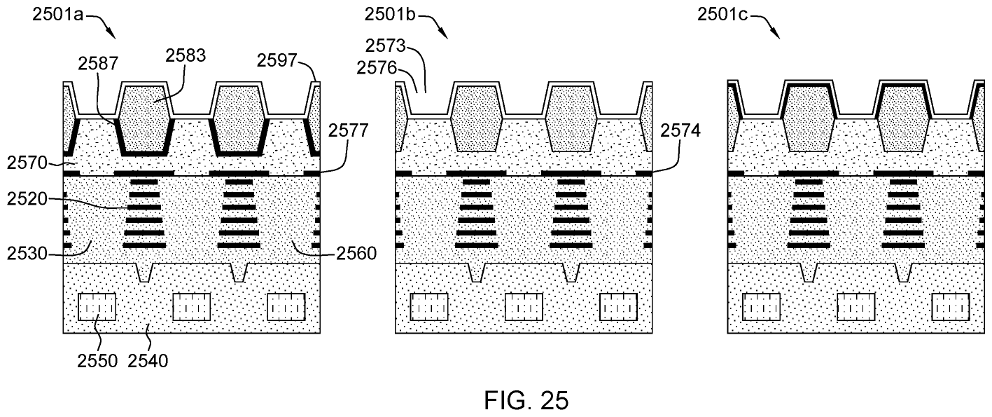

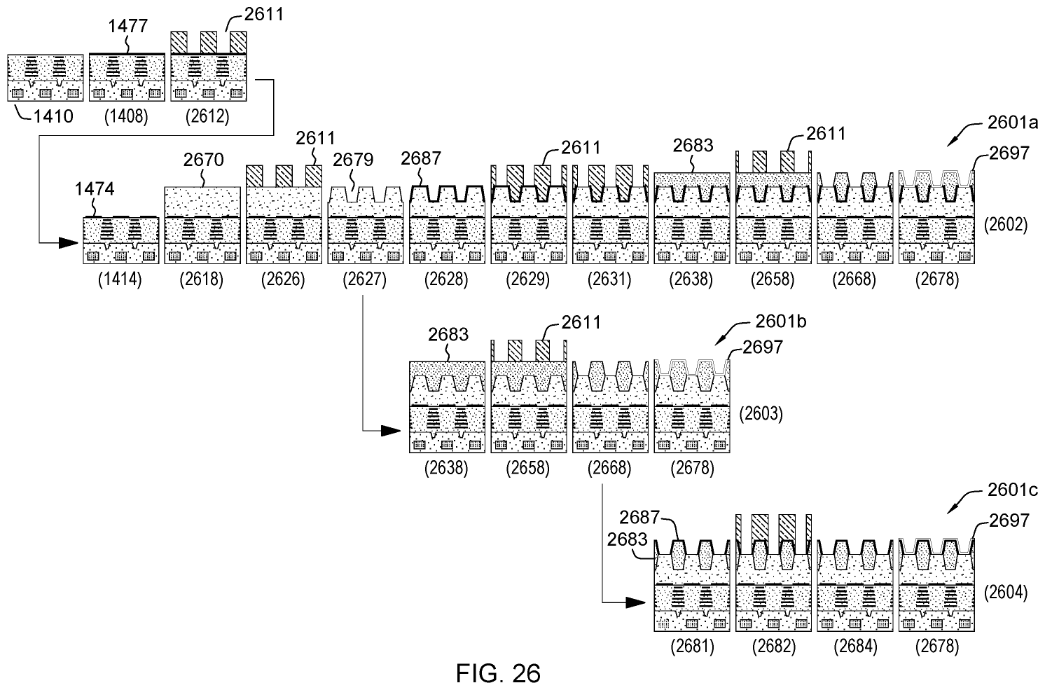

FIGS. 23 and 25 illustrate six different examples of biosensors that include FSI sensors. In each of these examples, the biosensors include an etched layer, that includes germanium. FIGS. 24 and 26 illustrate examples of methods utilized to make the sensors in FIGS. 23 and 25.

FIG. 23 illustrates three configurations for a biosensor 2301a-2301c where the variation in each is the placement and/or the exclusion of a conductive layer 2387. In one example 2301a, a conductive layer 2387 (e.g., metal) is deposited on the germanium layer 2370 to limit crosstalk. The second example 2301b does not include this conductive layer 2387. Meanwhile, in the third example 2301c, a conductive layer 2387 is deposited on the second low index layer 2383 (e.g., an oxide layer). In each of these examples 2301a-2301c, in place of the deposited blanket germanium layer 1770 (FIG. 17), the germanium layer 2370 of FIG. 23 is an etched layer. The germanium layer 2370 in these biosensors 2301a-2301c includes trenches 2379 in the germanium layer 2370. Parts of the second low (refractive) index layer (e.g., oxide) 2383 and/or a conductive layer 2387 fill these trenches 2379. In each case, the biosensor 2301a-2303c includes a sensor 2310, in these examples, an FSI sensor. The sensor 2310 includes a substrate 2340 comprising one or more diodes 2350 and a low (refractive) index layer 2360 (e.g., oxide). The low index layer 2360 includes electrically conductive materials 2320 (e.g., metal).

Each of the biosensors 2301a-2301c includes an etched germanium layer 2370 formed over a top surface of the sensor 2310. The germanium layer 2370 is etched to include trenches 2379. A second low (refractive) index layer (e.g., oxide) 2383 is formed over a top surface of the germanium layer 2370. Parts of the second low (refractive) index layer (e.g., oxide) 2383 fill the trenches 2379 in the germanium layer. The first example 2301a includes a conductive layer 2387 (e.g., metal) formed over a top surface of the germanium layer 2370, including in the trenches 2379, situated between the germanium layer 2370 and the second low (refractive) index layer 2383. The third example 2301c includes a conductive layer 2387 (e.g., metal) formed on the top surface of the germanium layer 2370. In these examples, the second low index layer 2383 includes trenches 2373. These trenches 2373 can form nanowells 2376. Each trench (e.g., nanowell) is positioned above at least one diode of the diodes 2350 (i.e., on a vertical axis extending from a bottom surface of the sensor 2310 to the top surface of the second low index layer 2383). Each biosensor 2301a-2301c is topped with a passivation layer 2397, which in these examples, can be comprised of silicon.

FIG. 24 illustrates workflows 2402-2404 that can be used to form the sensors 2301a-2301c of FIG. 23, labelled 2401a-2401c in FIG. 24. Each workflow in FIG. 24 commences with a sensor (e.g., a FSI CMOS) 2410. In the first workflow 2402, one forms a germanium layer 2470 over a top surface of the sensor 2410 (2418). One can form the germanium layer 2470 (e.g., SiGe) using a sputter technique. One can deposit a photoresist 2411 (e.g., utilizing photolithography) on a first portion of a top surface of the germanium layer 2470 (2426). Once the photoresist 2411 is deposited (e.g., utilizing photolithography) one can etch where the photoresist 2411 is not deposited to form trenches 2479 in the germanium layer 2470, and remove the photoresist (e.g., utilizing resist strips, chemical cleaning, and/or etching) (2427). In the first workflow 2402, one can form a conductive layer 2487 (e.g., metal) over a top surface of the germanium layer 2470 (e.g., using a technique including but not limited to, metal sputtering) (2428). In some examples, one removes certain portions of the conductive layer 2487 (the portions that do not line the trenches 2479), by depositing a photoresist 2411 on the top surface of the structure in the trenches 2479 (2429) and etching the exposed portions of the conductive layer 2487 (2431), exposing portions of the germanium layer 2470, and removing the photoresist 2411. After forming the conductive layer 2487 (e.g., via a metal sputter) in the first workflow 2402 and without forming this layer in the second and third workflows 2403-2404, one can form a second low index layer 2483 (e.g., an oxide layer) over a top surface of the germanium layer 2470 (which includes filling the trenches 2479 in the germanium layer 2470), and this top surface, in the first workflow 2402, includes the conductive layer 2487 (2438).

In examples that include nanowells 2476, one can deposit photoresist 2411 on a first portion of a top surface of the second low index layer 2483 (2458) (e.g., using photolithography). Portions of the surfaces upon which photoresist 2411 is deposited are preserved during a subsequent etching process. In these examples, the photoresist 2411 is deposited on portions of the top surface of the second low index layer 2483 above (on a longitudinal axis) the trenches 2479 in the germanium layer 2470. One can then etch the portions of the second low index layer 2483 that are not covered by the photoresist 2411 (e.g., utilizing an oxide and metal etching process) to form trenches 2473 in the second low index layer 2483, exposing parts of the germanium layer 2470 (2468). One can also remove the photoresist 2411. In some examples, various chemistries can then be applied to the trenches 2473. A passivation layer 2497 can be deposited atop the top surface of the structure, which can be a silicon layer (2478).

In the third workflow 2404, before depositing the passivation layer 2497 (2478), one deposits a conductive layer 2487 on the second low index layer 2483 (which is already etched) (2481). This workflow 2404 includes covering a first portion of the conductive layer 2487 with a photoresist 2411 (2482). Where the photoresist is not covering the conductive layer 2487, one removes the conductive layer 2487 (e.g., using etching), and removes the photoresist (2484). Then, a passivation layer 2497 can be deposited atop the top surface of the structure, which can be a silicon layer (2478).

FIG. 25 includes three examples 2501a-2501c of biosensors that can be formed using the techniques described herein. The biosensors 2501a-2501c in FIG. 25 additionally include blocking (conductive) elements 2574 (e.g., metal) from a blocking (conductive) layer 2577 that is formed atop the sensor 2510 and then, partially removed (as illustrated and discussed in more detail in FIG. 26). As in the other examples, the sensors 2510 include a substrate 2540 comprising one or more diodes 2550 and a low (refractive) index layer 2560 (e.g., oxide). The low index layer 2560 includes electrically conductive materials 2520 (e.g., metal).

FIG. 26 illustrates workflows 2602-2604 that can be used to form the sensors 2501a-2501c of FIG. 25 these biosensors labelled 2601a-2601c in FIG. 26. Each workflow in FIG. 26 commences with a sensor (e.g., a FSI CMOS) 2610. A difference between these examples and the examples in FIG. 24 is that these workflows 2602-2604 include forming a conductive (e.g., metal) layer 2677 atop the sensor 2610 (2608). This layer 2677, a blocking layer, additionally contributes to crosstalk reduction. After forming the layer 2677 (e.g., via metal sputtering) (2608), one removes portions of the layer 2677 by forming a photoresist 2611 (e.g., using photolithography) on portions of the layer 2677 (2612) and removing, via etching, the portions of the layer 2677 that are not covered by the photoresist (2614). This removal leaves blocking (conductive) elements 2674 (e.g., metal) in locations below the bases of the nanowells 2676, in configurations that include nanowells 2676. One can remove the remaining photoresist 2611 before forming nanowells 2676.

Upon forming the blocking (conductive) elements 2674, these workflows 2602-2604 proceed similarly in the workflows 2402-2404 of FIG. 24. In the workflows 2602-2604, one forms a germanium layer 2670 over a top surface of the sensor 2610 (2618); this surface includes the blocking (conductive) elements 2674. One can form the germanium layer 2670 (e.g., SiGe) using a sputter technique. One can then deposit a photoresist 2611 (e.g., utilizing photolithography) on a first portion of a top surface of the germanium layer 2670 (2626). Once the photoresist 2611 has been deposited (e.g., utilizing photolithography) one can etch where the photoresist 2611 is not deposited to form trenches 2679 in the germanium layer and then, remove the photoresist 2611 (2627).

In the first workflow 2602, one can form a conductive layer 2687 (e.g., metal) over a top surface of the germanium layer 2670 (e.g., using a technique including but not limited to, metal sputtering) (2628). In some examples, one removes certain portions of the conductive layer 2687 (the portions that do not line the trenches 2679), by depositing a photoresist 2611 on the top surface of the structure in the trenches 2679 (2629) and etching the exposed portions of the conductive layer 2687 (2631), exposing portions of the germanium layer 2670. After forming the conductive layer 2687 (e.g., via a metal sputter) in the first workflow 2602 and without forming this layer in the second and third workflows 2603-2604, one can form a second low index layer 2683 (e.g., an oxide layer) over a top surface of the germanium later 2670 (which includes filling the trenches 2679 in the germanium layer 2670), and this top surface, in the first workflow 2602, includes the conductive layer 2687.

In examples that include nanowells 2676, one can deposit photoresist 2611 on a first portion of a top surface of the second low index layer 2683 (2658) (e.g., using photolithography). Portions of the surfaces upon which photoresist 2611 is deposited are preserved during a subsequent etching process. In these examples, the photoresist 2611 is deposited on portions of the top surface of the second low index layer 2683 above (on a longitudinal axis) the trenches 2679 in the germanium layer 2670. One can then etch the portions of the second low index layer 2683 that are not covered by the photoresist 2611 (e.g., utilizing an oxide and metal etching process) to form trenches 2673 in the second low index layer 2683, exposing parts of the germanium layer 2670 (2668). One can then remove the photoresist 2611. In some examples, various chemistries can then be applied to the trenches 2673. A passivation layer 2697 can be deposited atop the top surface of the structure, which can be a silicon layer (2678).

In the third workflow 2604, before depositing the passivation layer 2697 (2678), one deposits a conductive layer 2687 on the second low index layer 2683 (which is already etched) (2681). This workflow 2604 includes covering a first portion of the conductive layer 2687 with a photoresist 2611 (2682). Where the photoresist is not covering the conductive layer 2687, one removes the conductive layer 2687 (e.g., using etching), as well as the photoresist 2611 (following completion of the etching). Then, a passivation layer 2697 can be deposited atop the top surface of the structure, which can be a silicon layer (2678).

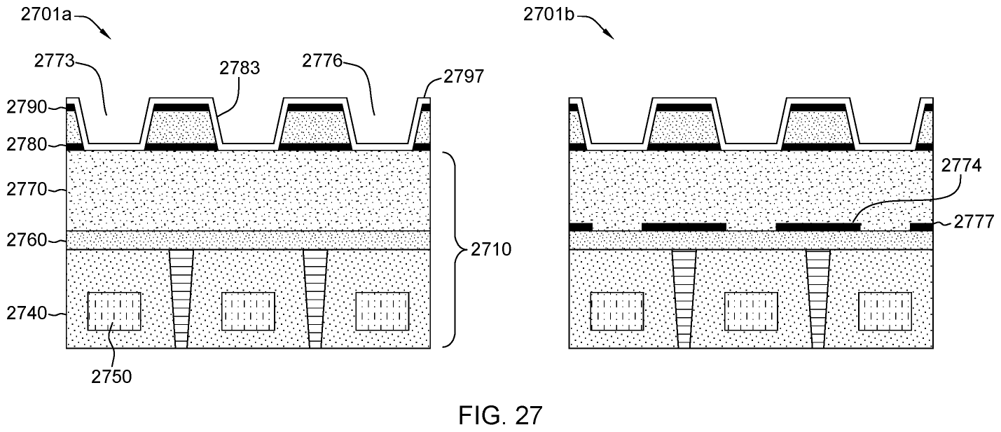

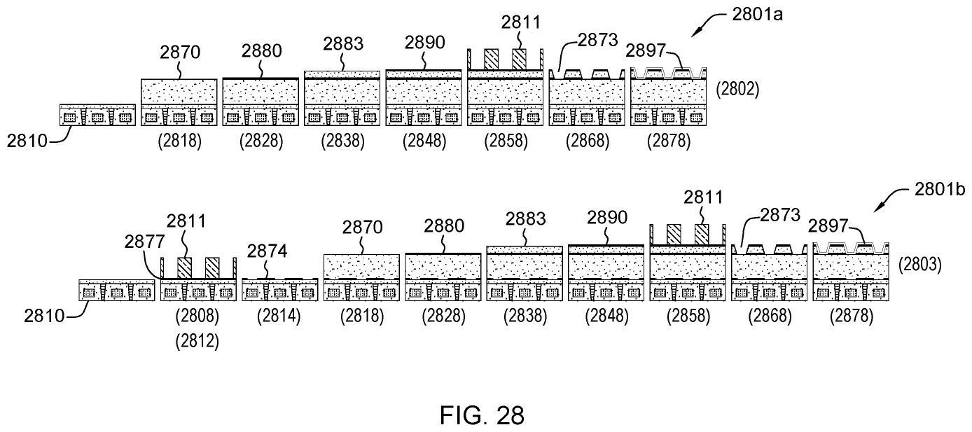

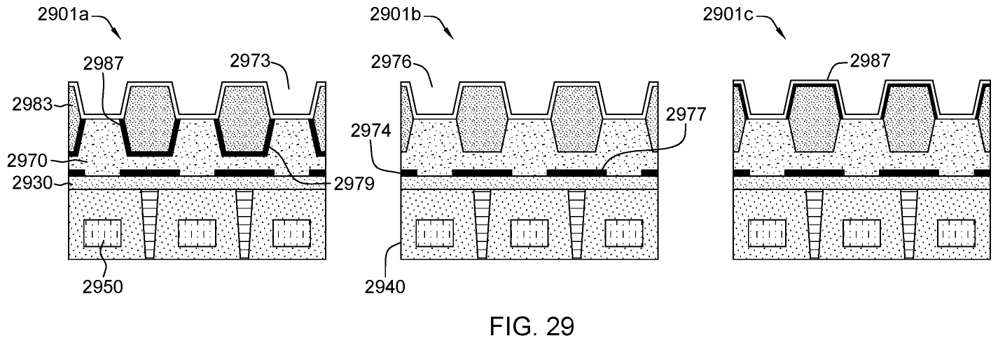

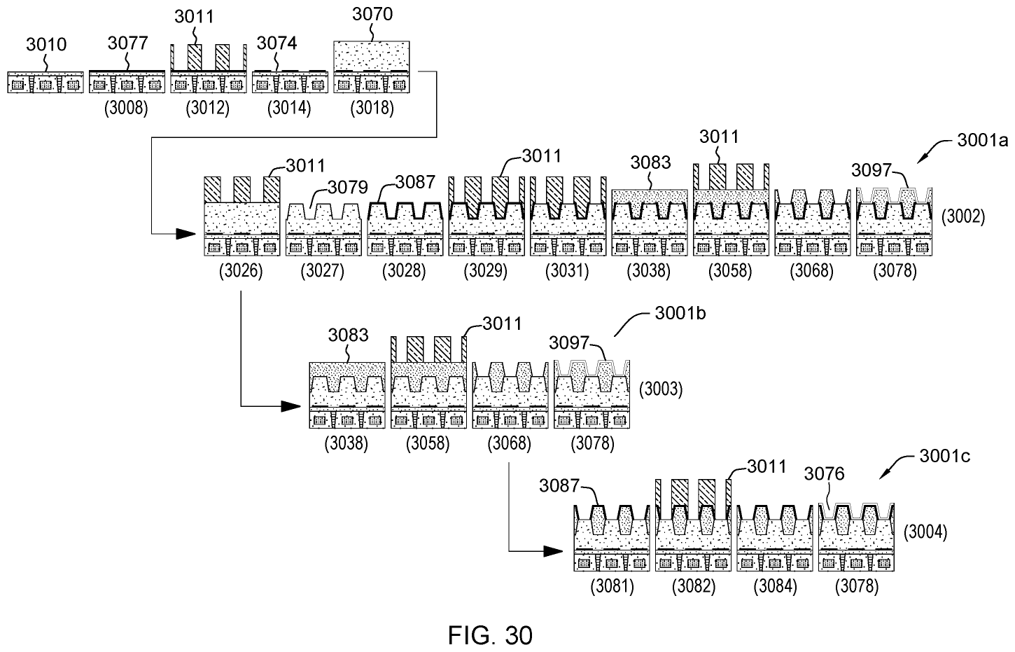

FIGS. 27-30 are illustrations of examples of biosensors and workflows utilized to form these sensors where the sensor elements are depicted as BSI sensors. Specifically, FIGS. 27 and 29 depict examples of the sensors 2701a-2701b and 2901a-2901c, while FIGS. 28 and 30 depict examples of respective workflows 2802-2803 and 2902-2904 to form these sensors. The first BSI-based biosensors discussed herein include blanket germanium layers while the second group include etched germanium layers. The difference between these designs is similar to the difference between the FSI-based examples discussed earlier.

Turning to FIG. 27, each of the biosensors 2701a-2701b includes a germanium layer 2770 formed over a surface that includes some of the low (refractive) index layer 2760 (e.g., oxide). In the second example 2701b, this surface includes both the low (refractive) index layer 2760 and conductive elements 2774 (left over from a (conductive) blocking layer 2777, which is illustrated in FIG. 30). The biosensors both include 2701a-2701b, a first conductive layer 2780 (e.g., metal) formed over a top surface of the germanium layer 2770, a second low (refractive) index layer (e.g., oxide) 2783 formed over a top surface of the first conductive layer 2780, and a second conductive layer 2790 (e.g., metal) formed over a top surface of the second low index layer 2783. In these examples, the second conductive layer 2790, the second low index layer 2783, and the first conductive layer 2780, include trenches 2773. These trenches can 2773 can form nanowells 2776. Each trench (e.g., nanowell) is positioned above at least one diode of the diodes 2750 (i.e., on a vertical axis extending from a bottom surface of the sensor 2710 to the top surface of the second low index layer 2783). Each biosensor 2701a-2701b is topped with a passivation layer 2797, which in these examples, can be comprised of silicon.

In FIG. 28, the workflow 2803 to form the second example 2801b (e.g., FIG. 27, 2701b) includes forming a blocking layer 2877, which is an additional conductive layer (e.g., metal) before forming the germanium layer 2870, while this aspect (and this layer) is omitted from the alternate workflow 2802 and the first example 2801a (e.g., FIG. 27, 2701a).

Each workflow in FIG. 28 commences with a sensor (e.g., a BSI CMOS) 2810. In the second workflow 2803, one forms a blocking layer 2877 (e.g., using a metal sputtering technique) over a top surface of the sensor 2810 (2808). One then can deposit photoresist 2811 (e.g., utilizing photolithography) atop portions of the blocking layer 2877 (2812). Utilizing a technique including but not limited to etching, including mechanical etching, one removes portions of the blocking layer 2877 (those not covered with the photoresist 2811), leaving blocking elements 2874 (which assist in crosstalk mitigation) and then, one removes the photoresist 2811 (e.g., utilizing resist strips, chemical cleaning, and/or etching) (2814).

Picking up the second workflow 2803 after forming the blocking elements 2874 and the first workflow 2802 at the beginning, one forms a germanium layer 2870 over a top surface of the sensor 2810 (which includes the blocking elements 2874 in the second workflow 2803) (2818). This germanium layer 2870 (e.g., SiGe) can be formed using a sputter technique. Once the germanium layer 2870 has been deposited (2818), one can form a first conductive layer 2880 (e.g., metal) over a top surface of the germanium layer 2870 (e.g., using a technique including but not limited to, metal sputtering) (2828). One can then form a second low index layer 2883 (e.g., an oxide layer) over a top surface of this first conductive layer 2880 (2838). Atop the second low index layer 2883, one can form a second conductive layer 2890 (2848).

Forming nanowells is optional (as discussed herein) and some examples will omit them, but for illustrative purposes, portions of this aspect are included in FIG. 28. To form nanowells 2876, one can deposit photoresist 2811 on a first portion of a top surface of the second conductive layer 2890 (2858) (e.g., using photolithography). Portions of the surfaces upon which photoresist 2811 is deposited are preserved during a subsequent etching process. One can then etch the portions of the second conductive 2890 layer that are not covered by the photoresist 2811 (e.g., utilizing an oxide and metal etching process) to form trenches 2873 in both conductive layers 2880, 2890 and the second low index layer 2883, exposing parts of the germanium layer 2870 (2868). One can then remove the photoresist 2811. In some examples, various chemistries can then be applied to the trenches. A passivation layer 2897 can be deposited atop the top surface of the structure, which can be a silicon layer (2878).

The biosensors 2901a-2901c in FIG. 29 are similar the biosensors those in FIG. 27, but additionally all include the aforementioned blocking (conductive) elements 2974 (e.g., metal) from a blocking (conductive) layer 2977 that is formed atop the sensor 2910, in these examples, a BSI sensor, and then, partially removed (as illustrated and discussed in more detail in FIG. 30). As in the other examples, the sensors 2910 include a substrate 2940 comprising one or more diodes 2950 and a low (refractive) index layer 2960 (e.g., oxide). The low index layer 2960 includes electrically conductive materials 2920 (e.g., metal). In each of these examples 2901a-2901c, in place of the deposited blanket germanium layer 2770 (FIG. 27) the germanium layer 2970 of FIG. 29 is an etched layer, in that in includes trenches 2979 in the germanium layer 2970 and parts of the second low (refractive) index layer (e.g., oxide) 2983 and/or a conductive layer 2987 fill these trenches 2979. The sensor 2910 includes a substrate 2940 comprising one or more diodes 2950 and a (refractive) index layer 2960 (e.g., oxide).

The workflows 3002-3004 all include forming a conductive (e.g., metal) layer 3077 atop the sensor 3010. This layer is a blocking layer that contributes to crosstalk reduction. Generally, FIG. 30 illustrates workflows 3002-3004 that can be used to form the sensors 2901a-2901c of FIG. 29 (labeled 3001a and 3001b in FIG. 30). Each workflow in FIG. 30 commences with a sensor (e.g., a BSI CMOS) 3010. After forming the conductive (e.g., metal) layer 3077 (e.g., via metal sputtering) (3008), one removes portions of the layer 3077 by forming a photoresist 3011 (e.g., using photolithography) on portions of the layer 3077 (3012) and removing, via etching, the portions of the layer 3077 that are not covered by the photoresist (3014). Once the etching is complete, one can remove the photoresist 3011 (e.g., utilizing resist strips, chemical cleaning, and/or etching). The removal of the portions of the layer 3077 leaves blocking (conductive) elements 3074 (e.g., metal) in locations below the bases of the nanowells 3076, in configurations that include nanowells 3076.

Upon forming the blocking (conductive) elements 3074, these workflows 3002-3004 proceed similarly in the workflows 2402-2404 of FIG. 24. In the workflows 3002-3004, one forms a germanium layer 3070 over a top surface of the sensor 3010 (3018); this surface includes the blocking (conductive) elements 3074. One can form the germanium layer 3070 (e.g., SiGe) using a sputter technique. One can then deposit a photoresist 3011 (e.g., utilizing photolithography) on a first portion of a top surface of the germanium layer 3070 (3026). Once the photoresist 3011 has been deposited (e.g., utilizing photolithography) one can etch where the photoresist 3011 is not deposited to form trenches 3079 in the germanium layer, and then remove the photoresist 3011 (e.g., utilizing resist strips, chemical cleaning, and/or etching) (3027).

In the first workflow 3002, one can form a conductive layer 3087 (e.g., metal) over a top surface of the germanium layer 3070 (e.g., using a technique including but not limited to, metal sputtering) (3028). In some examples, one removes certain portions of the conductive layer 3087 (the portions that do not line the trenches 3079), by depositing a photoresist 3011 on the top surface of the structure in the trenches 3079 (3029) and etching the exposed portions of the conductive layer 3087 (3031), exposing portions of the germanium layer 3070. After forming the conductive layer 3087 (e.g., via a metal sputter) in the first workflow 3002 and without forming this layer in the second and third workflows 3003-3004, one can form a second low index layer 3083 (e.g., an oxide layer) over a top surface of the germanium layer 3070 (which includes filling the trenches 3079 in the germanium layer 3070), and this top surface, in the first workflow 3002, includes the conductive layer 3087.

In examples that include nanowells 3076, one can deposit photoresist 3011 on a first portion of a top surface of the second low index layer 3083 (3058) (e.g., using photolithography). Portions of the surfaces upon which photoresist 3011 is deposited are preserved during a subsequent etching process. In these examples, the photoresist 3011 is deposited on portions of the top surface of the second low index layer 3083 above (on a longitudinal axis) the trenches 3079 in the germanium layer 3070. One can then etch the portions of the second low index layer 3083 that are not covered by the photoresist 3011 (e.g., utilizing an oxide and metal etching process) to form trenches 3073 in the second low index layer 3083, exposing parts of the germanium layer 3070 (3068). One can then remove the remaining photoresist 3011. In some examples, various chemistries can then be applied to the trenches 3073. A passivation layer 3097 can be deposited atop the top surface of the structure, which can be a silicon layer (3078).

In the third workflow 3004, before depositing the passivation layer 3097 (3078), one deposits a conductive layer 3087 on the second low index layer 3083 (which is already etched) (3081). This workflow 3004 includes covering a first portion of the conductive layer 3087 with a photoresist 3011 (3082). Where the photoresist is not covering the conductive layer 3087, one removes the conductive layer 3087 (e.g., using etching) and then removes the photoresist 3011 (e.g., utilizing resist strips, chemical cleaning, and/or etching). Then, a passivation layer 3097 can be deposited atop the top surface of the structure, which can be a silicon layer (3078).

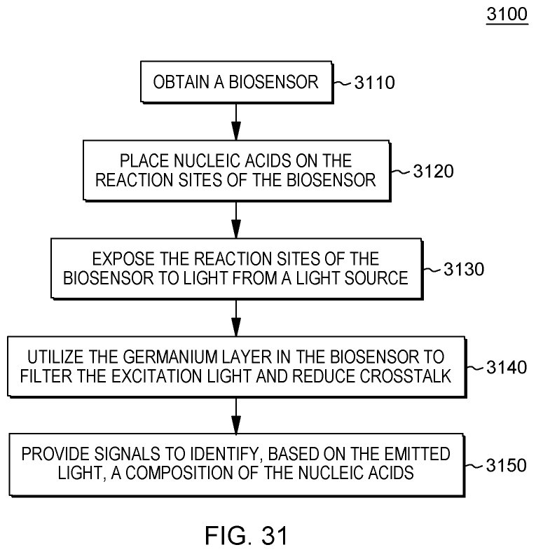

Each apparatus described herein can be utilized as a biosensor. FIG. 31 provides as illustration of aspects of various workflows 3100 that include utilizing various examples of the apparatuses described herein. Thus, for each apparatus described herein, one can obtain the biosensor (e.g., included in apparatuses described herein) (3110). The biosensor, as discussed and illustrated in the accompanying figures, each include an image sensor with one or more diodes and a germanium layer formed on (and sometimes in part of) the image sensor. The biosensor includes wells and reaction sites. One can place one or more nucleic acid on the reaction sites (3120), expose the reaction sites of the biosensor to light from a light source (the light from the light source comprises excitation light) (3130). In some examples, the image sensor receives emitted light from the reaction sites, via the germanium layer. The germanium layer filters the excitation light from the light and reduces crosstalk (e.g., associated with the emitted light) (3140). The image sensor can then provide signals that are used to identify, based on the emitted light, a composition of the nucleic acids (3150). In obtaining this emitted light, in these examples, the biosensor can structures propagate the emitted light through the germanium layer to reach at least one diode of the one or more diodes. In some examples, the reaction sites comprise fluorophores. In these examples, based on exposing the reaction sites of the sensor to light from a light source, the excitation light causes the fluorophores to emit the emitted light.

Described herein are various examples of forming biosensors, utilizing biosensors, and descriptions of structures of various biosensors. Various examples of the methods and the apparatuses are described below.

In some examples herein, the method comprises: forming one or more diodes on a first surface of a substrate, wherein the first surface of the substrate is parallel to a second surface of the substrate; forming one or more trenches between the one or more diodes, the one or more trenches extending toward the second surface of the substrate from the first surface of the substrate, wherein the forming comprises filling the one or more trenches and planarizing the one or more filled trenches to form a first surface substantially parallel to a first surface of the one or more diodes and the first surface of the substrate; removing a portion of the substrate such that the one or more trenches extend through the substrate from the first surface of the substrate to the second surface of the substrate; bonding a carrier wafer to the second surface of the substrate; forming a germanium layer above the second surface of the substrate; and forming a dielectric stack above a surface of the germanium layer.

In some examples of the method, forming the one or more trenches comprises etching the one or more trenches in the substrate.

In some examples of the method, the substrate comprises silicon.

In some examples of the method, filling the one or more trenches comprises filling the one or more trenches with one or more dielectric layers.

In some examples of the method, the dielectric stack comprises one or more nanowells.

In some examples of the method, forming the germanium layer on the second surface of the substrate comprises depositing germanium on the second surface of the substrate.

In some examples of the method, a technique for the depositing is selected from the group consisting of: Plasma Enhanced Chemical Vapor Deposition (PECVD), sputter, e-beam evaporation, crystalline growth and etching, and radical activation bonding in vacuum.

In some examples of the method, the selected technique is crystalline growth and etching and the crystalline growth and etching comprises one of: transfer wafer bonding or direct wafer bonding.

In some examples of the method, forming the germanium layer above the second surface of the substrate further comprises: forming a first one or more dielectric layers on the second surface of the substrate; forming the germanium layer on a surface of the one or more dielectric layers; and forming a second one or more dielectric layers on a surface of the germanium layer.

In some examples of the method, the substrate, one or more diodes, the carrier wafer, and the one or more filled trenches comprise a sensor.

In some examples of the method, the sensor comprises a complementary metal-oxide-semiconductor (CMOS).

In some examples of the method, the germanium layer comprises germanium and silicon.

In some examples of the method, the stack comprises: one or more dielectric layers; and a sensor compatible metal.

In some examples of the method, the germanium layer performs loss induced crosstalk reduction and filters out excitation light when light source emits light toward the dielectric stack.

In some examples of the method, based on the loss induced crosstalk reduction, a signal at neighboring pixels is substantially lower than a signal at paired pixels.

In some examples herein, the method comprises: forming a germanium layer above a top surface of an image sensor; and forming a dielectric stack above a top surface of the germanium layer.

In some examples of the method, the dielectric stack comprises one or more nanowells.

In some examples of the method, forming the germanium layer above the top surface of the image sensor further comprises: forming a first one or more dielectric layers on the top surface of the image sensor; forming the germanium layer on a surface of the one or more dielectric layers; and forming a second one or more dielectric layers on a surface of the germanium layer.

In some examples of the method, forming the germanium layer above the top surface of the image sensor comprises depositing germanium above the top surface of the image sensor.

In some examples of the method, a technique for the depositing is selected from the group consisting of: Plasma Enhanced Chemical Vapor Deposition (PECVD), sputter, e-beam evaporation, crystalline growth and etching, and radical activation bonding in vacuum.

In some examples of the method, the selected technique is crystalline growth and etching and the crystalline growth and etching comprises one of: transfer wafer bonding or direct wafer bonding.

In some examples of the method, the image sensor comprises a complementary metal-oxide-semiconductor (CMOS).

In some examples of the method, the image sensor comprises a backside image sensor with one or more deep trenches.

In some examples of the method, the germanium layer performs loss induced crosstalk reduction and filters out excitation light when light source emits light toward the dielectric stack.

In some examples herein, the method comprises: obtaining a biosensor, the biosensor comprising: a germanium layer above a top surface of an image sensor; and a dielectric stack above a top surface of the germanium layer, wherein the dielectric stack comprises wells and reaction sites; placing one or more nucleic acids in the reaction sites; and exposing the reaction sites of the biosensor to light from a light source, wherein the light comprises excitation light and emitted light; obtaining, by the image sensor, the emitted light, from the reaction sites, via the germanium layer, the emitted light, wherein the germanium layer filters the excitation light from the light and reduces crosstalk associated with the emitted light; and identifying, by the image sensor, based on the emitted light, a composition of the nucleic acids.

In some examples of the method, the image sensor comprises one or more diodes.

In some examples of the method, the obtaining the emitted light, from the reaction sites, via the germanium layer further comprises: propagating the emitted light through the germanium layer at non-vertical angles to reach at least one diode of the one or more diodes.

In some examples of the method, the reaction sites comprise fluorophores, and wherein based on exposing the reaction sites of the biosensor to light from a light source, the excitation light causes the fluorophores to emit the emitted light.

In some examples of the method, the germanium layer comprises germanium and silicon.

In some examples of the method, the biosensor further comprises: a first one or more dielectric layers on the top surface of the image sensor; and a second one or more dielectric layers on a surface of the germanium layer.

In some examples of the method, the image sensor comprises a complementary metal-oxide-semiconductor (CMOS).

In some examples of the method, the germanium layer comprises germanium and silicon.

In some examples of the method, the dielectric stack comprises: one or more dielectric layers; and a sensor compatible metal.

In some examples herein, the apparatus comprises: a filter layer comprising germanium; a flow channel floor defining a plurality of wells, wherein each well provides a reaction site, wherein the filter layer is positioned under flow channel floor, wherein the filter layer spans contiguously under the plurality of wells.

In some examples, of the apparatus, the apparatus further comprises: a plurality of sensors positioned under the filter layer, each sensor of the plurality of sensors centered under a corresponding well and reaction site, such that each sensor forms a sensing pair with a corresponding reaction site.

In some examples, of the apparatus, the filter layer further comprises silicon.

In some examples, of the apparatus, the filter layer has a height of approximately 300 micrometers to approximately 500 micrometers.

In some examples herein, a method for utilizing a biosensor (e.g., an apparatus) comprises: placing one or more nucleic acids in reaction sites of an apparatus, the apparatus comprising: a filter layer comprising germanium; a flow channel floor defining a plurality of wells, wherein each well provides a reaction site of the reaction sites, wherein the filter layer is positioned under flow channel floor; wherein the filter layer spans contiguously under the plurality of wells; exposing the reaction sites of the apparatus to light from a light source, wherein the light comprises excitation light and emitted light; receiving the emitted light from the reaction sites via the filter layer, wherein the filter layer filters the excitation light from the light and reduces crosstalk associated with the emitted light; and identifying, based on the emitted light, a composition of the one or more nucleic acids.

In some examples of the method, the apparatus includes a plurality of sensors positioned under the filter layer, each sensor of the plurality of sensors centered under a corresponding well and another reaction site of the reaction sites, such that each sensor forms a sensing pair with a corresponding reaction site.

In some examples of the method, the filter layer of the apparatus further comprises silicon.

In some examples of the method, the filter layer has a height of approximately 300 micrometers to approximately 500 micrometers.

In some examples herein, a method for forming aspects of a biosensor comprises: forming a germanium layer over a top surface of a sensor, wherein the sensor comprises: a substrate comprising one or more diodes; a first oxide layer formed over a top surface of the substrate; forming a first conductive layer over a top surface of the germanium layer; forming a second oxide layer over a top surface of the first conductive layer; forming a second conductive layer over a top surface of the second oxide layer; depositing photoresist on a first portion of a top surface of the second conductive layer; and etching through a second portion of the top surface of the second conductive layer, wherein the photoresist is not deposited on the second portion of the top surface of the second conductive layer, a portion of the second oxide layer, and a portion of the first conductive layer, wherein the etching forms one or more trenches, wherein the one or more trenches are each positioned above at least one diode of the one or more diodes, on a vertical axis extending from a bottom surface of the sensor to the top surface of the second oxide layer.

In some examples of the method, the germanium layer further comprises silicon, and forming the germanium layer comprises sputtering silicon-germanium onto the top surface of the first oxide layer.

In some examples of the method, the one or more trenches comprise nanowells.

In some examples of the method, forming the germanium layer over a top surface of a sensor further comprises: depositing photoresist on a first portion of the top surface of the first oxide layer; etching through a second portion of the top surface of the first oxide layer, wherein the photoresist is not deposited on the second portion of the top surface of the first oxide layer, wherein the etching forms one or more trenches in the first oxide layer; depositing a crosstalk mitigating substance above the first oxide layer, wherein the depositing fills the one or more trenches in the first oxide layer; planarizing the crosstalk mitigating substance such that a portion of the crosstalk mitigating substance forms a contiguous surface with the first portion of the top surface of the first oxide layer; and depositing a layer of silicon germanium on the top surface of the first oxide layer.

In some examples of the method, the crosstalk mitigating substance is selected from the group consisting of: oxide, nitride, and silicon.

In some examples of the method, the method also includes: forming a passivation layer over the first portion of the top surface of the second conductive layer.

In some examples of the method, forming the germanium layer over a top surface of a sensor further comprises: sputtering an additional conductive layer on the top surface of the first oxide layer; depositing photoresist on a first portion of the additional conductive layer, wherein the second portion of the first oxide layer is exposed; removing a second portion of the additional conductive layer with etching, wherein the photoresist is not deposited on the second portion of the additional conductive layer, wherein based on the removing, the top surface of the first oxide layer and the first portion of the additional conductive layer are exposed; and depositing a layer of silicon germanium on the top surface of the first oxide layer.

In some examples of the method, forming the germanium layer over a top surface of the sensor comprises: depositing photoresist on a first portion of the top surface of the first oxide layer; etching through a second portion of the top surface of the first oxide layer, wherein the photoresist is not deposited on the second portion of the top surface of the first oxide layer, wherein the etching forms one or more trenches in the first oxide layer; depositing the germanium layer above the first oxide layer, wherein the depositing partially fills the one or more trenches in the first oxide layer; depositing a crosstalk mitigating substance above the germanium layer, wherein the depositing fills a remainder of the one or more trenches in the first oxide layer; and planarizing the crosstalk mitigating substance such that the top surface of the germanium layer is a contiguous surface comprising a portion of the crosstalk mitigating substance and the first portion of the top surface of the first oxide layer.

In some examples of the method, the crosstalk mitigating substance is selected from the group consisting of: oxide, nitride, and silicon.

In some examples of the method, the sensor is a front-side illuminated complementary metal-oxide semiconductor.

In some examples of the method, the method includes: forming a silicon layer on the top surface of the second oxide layer.

In some examples of the method, the first conductive layer and the second conductive layer are comprised of metal.

In some examples of the method, the first oxide layer comprises electrically conductive materials.

In some examples of the method, the sensor is a back-side illuminated complementary metal-oxide semiconductor.

In some examples herein, a method for forming aspects of a biosensor comprises: forming a germanium layer over a top surface of a sensor, wherein the sensor comprises: a substrate comprising one or more diodes; a first oxide layer formed over a top surface of the substrate, wherein forming the germanium layer comprises: depositing photoresist on a first portion of a top surface of the germanium layer; and etching through a second portion of the top surface of the germanium layer, wherein the photoresist is not deposited on the second portion of the top surface of the germanium, wherein the etching forms one or more trenches, wherein the trenches are each positioned above a space between at least one diode and another diode of the one or more diodes, on a vertical axis extending from a bottom surface of the sensor to the top surface of the germanium layer; forming a second oxide layer over a top surface of the germanium layer; depositing photoresist on a first portion of a top surface of the second oxide layer; and etching through a second portion of the top surface of the second oxide layer, wherein the photoresist is not deposited on the second portion of the top surface of the second oxide layer, wherein the etching forms an additional one or more trenches, wherein the additional one or more trenches are each positioned above at least one diode of the one or more diodes, on a vertical axis extending from a bottom surface of the sensor to the top surface of the second oxide layer.

In some examples of the method, the method includes: forming a silicon layer over the top surface of the second oxide layer.

In some examples of the method, forming the germanium layer over the top surface of the sensor further comprises: depositing silicon germanium on the top surface of the first oxide layer; depositing a conductive layer on the silicon germanium; and depositing photoresist on a first portion of the conductive layer; and etching through a second portion of the conductive layer, wherein the photoresist is not deposited on the second portion of the conductive layer, wherein the etching removes the second portion of the conductive layer, and wherein the top surface germanium layer comprises a surface comprising a portion of the silicon germanium and the first portion of the conductive layer.

In some examples of the method, the method includes: depositing a conductive layer on the top surface of the second oxide layer; depositing photoresist on a first portion of the conductive layer; and etching through a second portion of the conductive layer, wherein the photoresist is not deposited on the second portion of the conductive layer, wherein the etching removes the second portion of the conductive layer.

In some examples of the method, forming the germanium layer over the top surface of the sensor further comprises: depositing silicon germanium on the top surface of the first oxide layer; depositing a conductive layer on the top surface of the silicon germanium; depositing photoresist on a first portion of the conductive layer; and etching through a second portion of the conductive layer, wherein the photoresist is not deposited on the second portion of the conductive layer, wherein the etching removes the second portion of the conductive layer, and where the top surface of the germanium layer comprises the first portion of the conductive layer and a portion of the silicon germanium.

In some examples of the method, forming the germanium layer over the top surface of the sensor further comprises: depositing a conductive layer on the top surface of the sensor; depositing photoresist on a first portion of the conductive layer; and etching through a second portion of the conductive layer, wherein the photoresist is not deposited on the second portion of the conductive layer, wherein the etching removes the second portion of the conductive layer; and depositing silicon germanium over a portion of the first oxide layer and the first portion of the conductive layer.

In some examples of the method, the sensor is a front-side illuminated complementary metal-oxide semiconductor.

In some examples of the method, the sensor is a back-side illuminated complementary metal-oxide semiconductor.

In some examples of the method, the first oxide layer comprises conductive components.

In some examples herein, an apparatus including a biosensor comprises: a sensor comprising: a substrate comprising one or more diodes; a first oxide layer formed over a top surface of the substrate; a germanium layer formed over a top surface of the sensor; a first conductive layer formed over a top surface of the germanium layer; a second oxide layer formed over a top surface of the first conductive layer; a second conductive layer formed over a top surface of the second oxide layer, wherein the second conductive layer, the second oxide layer, and the first conductive layer comprise one or more trenches, and wherein the one or more trenches are each positioned above at least one diode of the one or more diodes, on a vertical axis extending from a bottom surface of the sensor to the top surface of the second oxide layer.

In some examples of the apparatus, the germanium layer further comprises silicon.

In some examples of the apparatus, the one or more trenches comprise nanowells.

In some examples of the apparatus, the first oxide layer comprises an oxide substance and a crosstalk mitigating substance, wherein the crosstalk mitigating substance fills trench structures in the oxide substance.

In some examples of the apparatus, the crosstalk mitigating substance is selected from the group consisting of: oxide, nitride, and silicon.

In some examples of the apparatus, the apparatus includes: a passivation layer formed over the first portion of the top surface of the second conductive layer.

In some examples of the apparatus, the germanium layer comprises: an additional conductive layer over the top surface of the first oxide layer, wherein the additional and conductive layer comprises fissures; and a layer of silicon germanium over the top surface of the first oxide layer.

In some examples of the apparatus, the first oxide layer comprises trench structures, wherein the top surface of the sensor is an uneven surface and the germanium layer formed over a top surface of the sensor comprises: silicon germanium filling a portion of the trench structures; and a crosstalk mitigating substance filling a remainder of the trench structures, wherein the top surface of the germanium layer is a contiguous surface comprising a portion of the crosstalk mitigating substance and a portion of a top surface of the first oxide layer.

In some examples of the apparatus, the crosstalk mitigating substance filling the remainder of the trench structures is selected from the group consisting of: oxide, nitride, and silicon.

In some examples of the apparatus, the sensor is a front-side illuminated complementary metal-oxide semiconductor.

In some examples of the apparatus, the oxide layer comprises electrically conductive materials.

In some examples of the apparatus, the apparatus includes: a silicon layer on the top surface of the second oxide layer.

In some examples of the apparatus, the first conductive layer and the second conductive layer are comprised of metal.

In some examples of the apparatus, the sensor is a back-side illuminated complementary metal-oxide semiconductor.

In some examples of the apparatus, the apparatus includes a pixel pitch of less than one micron.

In some examples of the apparatus, the germanium layer is of a thickness of less than 400 nm.

In some examples of the apparatus, the germanium layer is of a thickness between approximately 300 nm and approximately 330 nm.

In some examples herein, an apparatus including a biosensor comprises: a sensor comprising: a substrate comprising one or more diodes; and a first oxide layer formed over a top surface of the substrate; a germanium layer over a top surface of a sensor, wherein the germanium layer comprises one or more trenches positioned above a space between at least one diode and another diode of the one or more diodes, on a vertical axis extending from a bottom surface of the sensor to the top surface of the germanium layer; and a second oxide layer over a top surface of the germanium layer, wherein the second oxide layer fills the trenches in the germanium layer, wherein the second oxide layer comprises one or more trenches, each trench in the second oxide layer positioned above at least one diode of the one or more diodes, on a vertical axis extending from a bottom surface of the sensor to a top surface of the second oxide layer, wherein the trenches in the second oxide layer expose portions of the germanium layer.

In some examples of the apparatus, the apparatus includes: a silicon layer over the top surface of the second oxide layer.

In some examples of the apparatus, the apparatus includes: a conductive layer comprising lining the one or more trenches in the germanium layer.

In some examples of the apparatus, the apparatus includes: a conductive layer over the top surface of the second oxide layer.

In some examples of the apparatus, the apparatus includes: a conductive layer over the top surface of the sensor.

In some examples of the apparatus, the sensor is a front-side illuminated complementary metal-oxide semiconductor.

In some examples of the apparatus, the first oxide layer comprises conductive components.

In some examples of the apparatus, the sensor is a back-side illuminated complementary metal-oxide semiconductor.

In some examples of the apparatus, the apparatus includes: a pixel pitch of less than one micron.

In some examples of the apparatus, the germanium layer is of a thickness of less than 400 nm.

In some examples of the apparatus, the germanium layer is of a thickness of less than 300 nm.

In some examples of the apparatus, the germanium layer is of a thickness between approximately 300 nm and approximately 330 nm.

In some examples herein, a method for utilizing a biosensor comprises: placing one or more nucleic acids in reaction sites of a sensor, the sensor comprising: a substrate comprising one or more diodes; and a first oxide layer formed over a top surface of the substrate; a germanium layer over a top surface of the first oxide layer, wherein the germanium layer comprises one or more trenches positioned above a space between at least one diode and another diode of the one or more diodes, on a vertical axis extending from a bottom surface of the sensor to the top surface of the germanium layer; and a second oxide layer over a top surface of the germanium layer, wherein the second oxide layer fills the trenches in the germanium layer, wherein the second oxide layer comprises one or more trenches, each trench in the second oxide layer positioned above at least one diode of the one or more diodes, on a vertical axis extending from a bottom surface of the sensor to a top surface of the second oxide layer, wherein the trenches in the second oxide layer expose portions of the germanium layer, wherein the second oxide layer comprises wells and the reaction sites; exposing the reaction sites of the sensor to light from a light source, wherein the light comprises excitation light and emitted light; receiving, by the one or more diodes, the emitted light from the reaction sites via the germanium layer, wherein the germanium layer filters the excitation light from the light and reduces crosstalk associated with the emitted light; and identifying, based on the emitted light, a composition of the nucleic acids.

In some examples of the method, the sensor further comprises: a conductive layer on the top surface of the sensor.

In some examples of the method, receiving the emitted light from the reaction sites via the germanium layer further comprises: propagating the emitted light through the germanium layer to reach at least one diode of the one or more diodes.

In some examples of the method, the reaction sites comprise fluorophores, and wherein based on exposing the reaction sites of the sensor to light from a light source, the excitation light causes the fluorophores to emit the emitted light.

In some examples of the method, the germanium layer comprises germanium and silicon.

In some examples of the method, the sensor is a front-side illuminated complementary metal-oxide semiconductor.

In some examples of the method, the sensor is a back-side illuminated complementary metal-oxide semiconductor.

In some examples of the method, the sensor further comprises: a silicon layer over the top surface of the second oxide layer.

In some examples of the method, the sensor further comprises: a conductive layer comprising lining the one or more trenches in the germanium layer.

In some examples of the method, the sensor further comprises: a conductive layer over the top surface of the second oxide layer.

In some examples herein, a method of utilizing a biosensor comprises: placing one or more nucleic acids in reaction sites of a biosensor, the biosensor comprising: a sensor, the sensor comprising: a substrate comprising one or more diodes; and a first oxide layer formed over a top surface of the substrate; a germanium layer formed over a top surface of the sensor; a first conductive layer formed over a top surface of the germanium layer; a second oxide layer formed over a top surface of the first conductive layer; a second conductive layer formed over a top surface of the second oxide layer, wherein the second conductive layer, the second oxide layer, and the first conductive layer comprise one or more trenches, and wherein the one or more trenches are each positioned above at least one diode of the one or more diodes, on a vertical axis extending from a bottom surface of the sensor to the top surface of the second oxide layer, wherein the trenches comprise wells and reaction sites; exposing the reaction sites of the biosensor to light from a light source, wherein the light comprises excitation light; receiving, by the one or more diodes, the emitted light from the reaction sites via the germanium layer, wherein the germanium layer filters the excitation light and reduces crosstalk associated with the emitted light; and identifying, based on the emitted light, a composition of the one or more nucleic acids.

In some examples of the method, the germanium layer further comprises silicon.

In some examples of the method, the first oxide layer comprises an oxide substance and a crosstalk mitigating substance, and the crosstalk mitigating substance fills trench structures in the oxide substance.

In some examples of the method, the crosstalk mitigating substance is selected from the group consisting of: oxide, nitride, and silicon.

In some examples of the method, the biosensor further comprises: a passivation layer formed over the first portion of the top surface of the second conductive layer.

In some examples of the method, the germanium layer comprises: an additional conductive layer over the top surface of the first oxide layer, wherein the additional and conductive layer comprises fissures; and a layer of silicon germanium over the top surface of the first oxide layer.

In some examples of the method, the first oxide layer comprises trench structures, wherein the top surface of the sensor is an uneven surface and the germanium layer formed on a top surface of the sensor comprises: silicon germanium filling a portion of the trench structures; and a crosstalk mitigating substance filling a remainder of the trench structures, wherein the top surface of the germanium layer is a contiguous surface comprising a portion of the crosstalk mitigating substance and a portion of a top surface of the first oxide layer.

In some examples of the method, the crosstalk mitigating substance filling the remainder of the trench structures is selected from the group consisting of: oxide, nitride, and silicon.

In some examples of the method, the sensor is a front-side illuminated complementary metal-oxide semiconductor.

In some examples of the method, the oxide layer comprises electrically conductive materials.

In some examples of the method, the sensor is a back-side illuminated complementary metal-oxide semiconductor.

In some examples of the method, the obtaining the emitted light, from the reaction sites, via the germanium layer further comprises: propagating the emitted light through the germanium layer to reach at least one diode of the one or more diodes.

In some examples of the method, the reaction sites comprise fluorophores, and wherein based on exposing the reaction sites of the sensor to light from a light source, the excitation light causes the fluorophores to emit the emitted light.

The flowchart and block diagrams in the Figures illustrate the architecture, functionality, and operation of possible implementations of systems, methods, and computer program products according to various examples of the present implementation. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular examples only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, processes, operations, elements, and/or components, but do not preclude the presence or addition of one or more other features, integers, steps, processes, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more examples has been presented for purposes of illustration and description but is not intended to be exhaustive or limited to in the form disclosed. Many modifications and variations will be apparent to those of ordinary skill in the art. The example was chosen and described in order to best explain various aspects and the practical application, and to enable others of ordinary skill in the art to understand various examples with various modifications as are suited to the particular use contemplated.

It should be appreciated that all combinations of the foregoing concepts and additional concepts discussed in greater detail below (provided such concepts are not mutually inconsistent) are contemplated as being part of the subject matter disclosed herein at least to achieve the benefits as described herein. In particular, all combinations of claims subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

This written description uses examples to disclose the subject matter, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the above description is intended to be illustrative, and not restrictive. For example, the above-described examples (and/or aspects thereof) may be used in combination with each other. In addition, many modifications may be made to adapt a particular situation or material to the teachings of the various examples without departing from their scope. While the dimensions and types of materials described herein are intended to define the parameters of the various examples, they are by no means limiting and are merely provided by way of example. Many other examples will be apparent to those of skill in the art upon reviewing the above description. The scope of the various examples should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Forms of term “based on” herein encompass relationships where an element is partially based on as well as relationships where an element is entirely based on. Forms of the term “defined” encompass relationships where an element is partially defined as well as relationships where an element is entirely defined. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure. It is to be understood that not necessarily all such objects or advantages described above may be achieved in accordance with any particular example. Thus, for example, those skilled in the art will recognize that the systems and techniques described herein may be embodied or carried out in a manner that achieves or optimizes one advantage or group of advantages as taught herein without necessarily achieving other objects or advantages as may be taught or suggested herein.

While the subject matter has been described in detail in connection with only a limited number of examples, it should be readily understood that the subject matter is not limited to such disclosed examples. Rather, the subject matter can be modified to incorporate any number of variations, alterations, substitutions or equivalent arrangements not heretofore described, but which are commensurate with the spirit and scope of the subject matter. Additionally, while various examples of the subject matter have been described, it is to be understood that aspects of the disclosure may include only some of the described examples. Also, while some examples are described as having a certain number of elements it will be understood that the subject matter can be practiced with less than or greater than the certain number of elements. Accordingly, the subject matter is not to be seen as limited by the foregoing description but is only limited by the scope of the appended claims.

1-41. (canceled)

42. A method comprising:
forming a germanium layer over a top surface of a sensor, wherein the sensor comprises:
a substrate comprising one or more diodes;
a first oxide layer formed over a top surface of the substrate;
forming a first conductive layer over a top surface of the germanium layer;
forming a second oxide layer over a top surface of the first conductive layer;
forming a second conductive layer over a top surface of the second oxide layer;
depositing photoresist on a first portion of a top surface of the second conductive layer; and
etching through a second portion of the top surface of the second conductive layer, wherein the photoresist is not deposited on the second portion of the top surface of the second conductive layer, a portion of the second oxide layer, and a portion of the first conductive layer, wherein the etching forms one or more trenches, wherein the one or more trenches are each positioned above at least one diode of the one or more diodes, on a vertical axis extending from a bottom surface of the sensor to the top surface of the second oxide layer.

43. The method of claim 42, wherein the germanium layer further comprises silicon, and wherein forming the germanium layer comprises sputtering silicon-germanium onto the top surface of the first oxide layer.

44. The method of claim 42, wherein the one or more trenches comprise nanowells.

45. The method of claim 42, wherein forming the germanium layer over a top surface of a sensor further comprises:
depositing photoresist on a first portion of the top surface of the first oxide layer;
etching through a second portion of the top surface of the first oxide layer, wherein the photoresist is not deposited on the second portion of the top surface of the first oxide layer, wherein the etching forms one or more trenches in the first oxide layer;
depositing a crosstalk mitigating substance above the first oxide layer, wherein the depositing fills the one or more trenches in the first oxide layer;
planarizing the crosstalk mitigating substance such that a portion of the crosstalk mitigating substance forms a contiguous surface with the first portion of the top surface of the first oxide layer; and
depositing a layer of silicon germanium on the top surface of the first oxide layer.

46. (canceled)

47. (canceled)

48. The method of claim 42, wherein forming the germanium layer over a top surface of a sensor further comprises:
sputtering an additional conductive layer on the top surface of the first oxide layer;
depositing photoresist on a first portion of the additional conductive layer, wherein the second portion of the first oxide layer is exposed;
removing a second portion of the additional conductive layer with etching, wherein the photoresist is not deposited on the second portion of the additional conductive layer, wherein based on the removing, the top surface of the first oxide layer and the first portion of the additional conductive layer are exposed; and
depositing a layer of silicon germanium on the top surface of the first oxide layer.

49. The method of claim 42, wherein forming the germanium layer over a top surface of the sensor comprises:
depositing photoresist on a first portion of the top surface of the first oxide layer;
etching through a second portion of the top surface of the first oxide layer, wherein the photoresist is not deposited on the second portion of the top surface of the first oxide layer, wherein the etching forms one or more trenches in the first oxide layer;
depositing the germanium layer above the first oxide layer, wherein the depositing partially fills the one or more trenches in the first oxide layer;
depositing a crosstalk mitigating substance above the germanium layer, wherein the depositing fills a remainder of the one or more trenches in the first oxide layer; and
planarizing the crosstalk mitigating substance such that the top surface of the germanium layer is a contiguous surface comprising a portion of the crosstalk mitigating substance and the first portion of the top surface of the first oxide layer.

50. (canceled)

51. The method of claim 42, wherein the sensor is a front-side illuminated complementary metal-oxide semiconductor.

52. (canceled)

53. (canceled)

54. (canceled)

55. The method of claim 42, wherein the sensor is a back-side illuminated complementary metal-oxide semiconductor.

56-64. (canceled)

65. An apparatus comprising:
a sensor comprising:
a substrate comprising one or more diodes;
a first oxide layer formed over a top surface of the substrate;
a germanium layer formed over a top surface of the sensor;
a first conductive layer formed over a top surface of the germanium layer;
a second oxide layer formed over a top surface of the first conductive layer;
a second conductive layer formed over a top surface of the second oxide layer, wherein the second conductive layer, the second oxide layer, and the first conductive layer comprise one or more trenches, and wherein the one or more trenches are each positioned above at least one diode of the one or more diodes, on a vertical axis extending from a bottom surface of the sensor to the top surface of the second oxide layer.

66. The apparatus of claim 65, wherein the germanium layer further comprises silicon.

67. The apparatus of claim 65, wherein the one or more trenches comprise nanowells.

68-93. (canceled)

94. A method comprising:
placing one or more nucleic acids in reaction sites of a sensor, the sensor comprising:
a substrate comprising one or more diodes; and
a first oxide layer formed over a top surface of the substrate;
a germanium layer over a top surface of the first oxide layer, wherein the germanium layer comprises one or more trenches positioned above a space between at least one diode and another diode of the one or more diodes, on a vertical axis extending from a bottom surface of the sensor to the top surface of the germanium layer; and
a second oxide layer over a top surface of the germanium layer, wherein the second oxide layer fills the trenches in the germanium layer, wherein the second oxide layer comprises one or more trenches, each trench in the second oxide layer positioned above at least one diode of the one or more diodes, on a vertical axis extending from a bottom surface of the sensor to a top surface of the second oxide layer, wherein the trenches in the second oxide layer expose portions of the germanium layer, wherein the second oxide layer comprises wells and the reaction sites;
exposing the reaction sites of the sensor to light from a light source, wherein the light comprises excitation light and emitted light;
receiving, by the one or more diodes, the emitted light from the reaction sites via the germanium layer, wherein the germanium layer filters the excitation light from the light and reduces crosstalk associated with the emitted light; and
identifying, based on the emitted light, a composition of the one or more nucleic acids.

95. The method of claim 94, the sensor further comprising:
a conductive layer on the top surface of the sensor.

96. The method of claim 95, wherein receiving the emitted light from the reaction sites via the germanium layer further comprises:
propagating the emitted light through the germanium layer to reach at least one diode of the one or more diodes.

97. The method of claim 94, wherein the reaction sites comprise fluorophores, and wherein based on exposing the reaction sites of the sensor to light from a light source, the excitation light causes the fluorophores to emit the emitted light.

98. The method of claim 94, wherein the germanium layer comprises germanium and silicon.

99. The method of claim 94, wherein the sensor is a front-side illuminated complementary metal-oxide semiconductor.

100. The method of claim 94, wherein the sensor is a back-side illuminated complementary metal-oxide semiconductor.

101. The method of claim 94, the sensor further comprising:
a silicon layer over the top surface of the second oxide layer.

102. The method of claim 94, the sensor further comprising:
a conductive layer comprising lining the one or more trenches in the germanium layer.

103-116. (canceled)
